(19) United States
(12) Patent Application Publication
(10) Pub. No.: US 2013/0004531 A1
(43) Pub. Date: Jan. 3, 2013

(54) IMMUNOGENS FROM UROPATHOGENIC ESCHERICHIA COLI

(75) Inventors: Francesco BERLANDA SCORZA, Siena (IT); Maria Rita Fontana, Siena (IT); Danilo Gomes Moriel, Siena (IT); Mariagrazia Pizza, Siena (IT); Laura Serino, Siena (IT); Jörg Hacker, Berlin (DE)

(73) Assignees: Robert Koch Institute, Berlin (DE); Novartis Vaccines and Diagnostics SRL, Siena (IT)

(21) Appl. No.: 13/603,391

(22) Filed: Sep. 4, 2012

Related U.S. Application Data
(62) Division of application No. 12/377,572, filed on Feb. 13, 2009.
(60) Provisional application No. 60/838,975, filed on Aug. 16, 2006.

(51) Int. Cl. C07K 14/245; C07K 7/06; A61P 31/04; A61K 39/108; A61P 37/04

(52) U.S. Cl. 424/190.1; 530/350; 530/328; 530/327; 530/326; 530/325; 530/324

(57) ABSTRACT

Disclosed herein are various polypeptides that can be included in immunogenic compositions specific for pathogenic E. coli strains. The polypeptides have cellular locations which render them accessible to the immune system. The genes encoding the polypeptides were initially identified as being present in uropathogenic strain 536 but absent from non-pathogenic strains.

### CROSS REFERENCE TO RELATED APPLICATIONS

This application is a Divisional of U.S. patent application Ser. No. 12/377,572, filed Feb. 13, 2009, which is the National Stage of International Patent Application of PCT/IB2007/003306, filed Aug. 15, 2007, which claims the benefit of U.S. provisional Patent Application Ser. No. 60/838,975, filed Aug. 16, 2006, each of which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 223002109410SeqListing.txt, date recorded: Sep. 4, 2012, size: 555 KB).

### TECHNICAL FIELD

This invention is in the field of Escherichia coli biology, and in particular relates to immunogens for use in immunising against extraintestinal pathogenic E. coli (ExPEC) strains.

### BACKGROUND OF THE INVENTION

Few microorganisms are as versatile as E. coli. As well as being an important member of the normal intestinal microflora of mammals, it has been widely exploited as a host in recombinant DNA technology. In addition, however, E. coli can also be a deadly pathogen.

E. coli strains have traditionally been classified as either commensal or pathogenic, and pathogenic strains are then sub-classified as intestinal or extraintestinal strains. More recent taxonomic techniques such as multilocus enzyme electrophoresis (MLEE) classify E. coli into five phylogenetic groups (A, B1, B2, D & E), and these groupings do not match the traditional ones. For instance, MLEE group B1 includes both commensal and pathogenic strains, and group D includes both intestinal and extraintestinal strains.

The extraintestinal pathogenic strains (or ‘ExPEC’ strains [1]) of E. coli fall into MLEE groups

B2 and D, and include both uropathogenic (UPEC) strains and meningitis/sepsis-associated (MNEC) strains. UPEC strains cause urinary tract infections (UTIs), and are the most common form of cystitis. They also cause pyelonephritis (and its complications such as sepsis) and catheter-associated infections. MNEC strains cause neonatal meningitis (0.1 cases per 1000 live births) with case fatality rates ranging from 25 to 40%, and are also responsible for around 1/6 of sepsis cases.

Most previous ExPEC vaccines have been based on cell lysates or on cellular structures. SOLCOUROVAC™ includes ten different heat-killed bacteria including six ExPEC strains, and a successful phase H clinical trial was reported in reference 2. URO-VAXOM™ is an oral tablet vaccine containing lyophilised bacterial lysates of 18 selected E. coli strains [3]. Baxter Vaccines developed a UTI vaccine based on pili from 6 to 10 different strains, but this product has been abandoned. Medlmmune developed a product called MEDI 516 based on the FimH^{+ve} adhesin complex [4], but phase II clinical trials shows inadequate efficacy. Moreover, there was a risk with this vaccine that it would also affect non-pathogenic FimH^{+ve} strains in the normal intestinal flora, and it was expected that this vaccine would be effective against UPEC strains only, because of its bladder-specific adherence mechanism, leaving other ExPEC strains uncontrolled.

There is thus a need for improved ExPEC vaccines, including a need to move away from crude cell lysates and towards better-defined molecules, and a need to identify further antigens that are suitable for inclusion in vaccines, particularly antigens that are prevalent among clinical ExPEC strains without also being found in commensal strains.

One way of addressing these needs was reported in reference 5, where the inventors looked for genes present in genomes of MLEE types B2 and D but absent from MLEE types A and B1. Further comparative approaches, based on subtractive hybridisation, were reported in references 6 and 7. Virulence genes in ExPEC strains have also been identified in reference 8. Reference 9 discloses an analysis of four pathogenicity islands in UPEC E. coli strain 536.

Reference 10 used the genome sequence of UPEC (O6:K2:H1) strain CFT073 [11,12] to identify sequences not present in non-pathogenic E. coli strains. Reference 13 discloses a comparison of the genome sequence of E. coli human pyelonephritis isolate 536 (O6:K15:H31), an UPEC, with sequence data for strains CFT073 (UPEC), EDL933 (enterohemorrhagic) and MG1655 (non-pathogenic laboratory strain). Genome sequences of pathogenic strains are available in the databases under accession numbers AE005174 (gi:56384585), BA000007 (gi:47118301) and NC-004431 (gi:26245917). A sequence from a non-pathogenic strain is available under accession number U00096 (gi:48994873).

It is an object of the invention to provide further antigens for use in immunisation against pathogenic E. coli strains, particularly ExPEC strains, and more particularly UPEC strains.

### SUMMARY OF THE INVENTION

The inventors have identified various genes that can be included in immunogenic compositions specific for pathogenic E. coli strains. The genes are from uropathogenic strains (UPEC) but are absent from non-pathogenic strains, and their encoded proteins have cellular locations which render them accessible to the immune system.

In one aspect, the invention relates to a polypeptide comprising: (a) an amino acid sequence selected from the group consisting of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166 and 167; (b) an amino acid sequence having at least 80% sequence identity to an amino acid sequence of (a); (c) an amino acid sequence which is a fragment of at least 10 consecutive amino acids from an amino acid sequence of (a); or (d) an amino acid sequence having at least 80% sequence identity to an amino acid sequence of (a) and including a fragment of at least 10 consecutive amino acids from an amino acid sequence of (a). In a particular embodiment, polypeptides of this aspect of the invention comprise a fragment which comprises at least one B-cell epitope of (a).

In another aspect, the invention relates to a polypeptide comprising: (a) an amino acid sequence selected from the group consisting of SEQ ID NOs 1, 2, 3, 4, 5, 6, and 7; (b) an amino acid sequence having at least 80% sequence identity to an amino acid sequence of (a); (c) an amino acid sequence which is a fragment of at least 10 consecutive amino acids from an amino acid sequence of (a); or (d) an amino acid sequence having at least 80% sequence identity to an amino acid sequence of (a) and including a fragment of at least 10 consecutive amino acids from an amino acid sequence of (a). In a particular embodiment, polypeptides of this aspect of the invention comprise a fragment which comprises at least one B-cell epitope of (a).

The present invention further relates to immunogenic compositions comprising one or more outer membrane vesicles (OMVs) expressing one or more polypeptides comprising: (a) an amino acid sequence selected from the group consisting of SEQ ID NOs 1, 2, 3, 4, 5, 6, and 7; (b) an amino acid sequence having at least 80% sequence identity to an amino acid sequence of (a); (c) an amino acid sequence which is a fragment of at least 10 consecutive amino acids from an amino acid sequence of (a); or (d) an amino acid sequence having at least 80% sequence identity to an amino acid sequence of (a) and including a fragment of at least 10 consecutive amino acids from an amino acid sequence of (a). In a particular embodiment, the immunogenic composition of this aspect of the invention comprises one or more polypeptides comprising a fragment which comprises at least one B-cell epitope of (a).

The polypeptides of the invention can be used in medicine and in the manufacture of a medicament for raising an immune response in a patient.

The present invention also relates to a pharmaceutical composition comprising a polypeptide of the invention in admixture with a pharmaceutically acceptable carrier. The invention further relates to a pharmaceutical composition comprising two or more polypeptides of the invention in admixture with a pharmaceutically acceptable carrier. In a particular embodiment, the pharmaceutical compositions of the invention further comprise a vaccine adjuvant.

The present invention also relates to methods for raising an immune response in a patient, comprising administering to the patient a pharmaceutical composition or immunogenic composition of the invention. In a particular embodiment, the immune response is protective against ExPEC infection.

Further aspects of the invention are described below.

### DETAILED DESCRIPTION OF THE INVENTION

The inventors have identified various and polypeptides that can be included in immunogenic compositions specific for pathogenic E. coli strains. The polypeptides have cellular locations which render them accessible to the immune system. The genes encoding the polypeptides were initially identified as being present in uropathogenic strain 536 but absent from non-pathogenic strains.

### Polypeptides

The invention provides polypeptides comprising the amino acid sequences disclosed in the examples. These amino acid sequences are given in the sequence listing as SEQ ID NOs 1 to 167. A preferred subset of SEQ ID NOs 1 to 167 is given in Table 2.

The invention also provides polypeptides comprising amino acid sequences that have sequence identity to the amino acid sequences disclosed in the examples (i.e. to SEQ ID NOs 1 to 168). Depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more). These polypeptides include homologs, orthologs, allelic variants and mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty=12 and gap extension penalty=1.

These polypeptide may, compared to the sequences of the examples, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may have one or more (e.g. 1, 2, 3, 4, 5, 6, 7, 8, 9, 10, etc.) single amino acid deletions relative to a reference sequence. The polypeptides may also include one or more (e.g. 1, 2, 3, 4, 5, 6, 7, 8, 9, 10, etc.) insertions (e.g. each of 1, 2, 3, 4 or 5 amino acids) relative to a reference sequence.

Preferred polypeptides include polypeptides that are lipidated, that are located in the outer membrane, that are located in the inner membrane, or that are located in the periplasm. Particularly preferred polypeptides are those that fall into more than one of these categories e.g. lipidated polypeptides that are located in the outer membrane. Lipoproteins may have a N-terminal cysteine to which lipid is covalently attached, following post-translational processing of the signal peptide.

The invention further provides polypeptides comprising fragments of the amino acid sequences disclosed in the examples. The fragments should comprise at least n consecutive amino acids from the sequences and, depending on the particular sequence, n is 7 or more (e.g. 8, 10, 12, 14, 16, 18, 20, 22, 24, 26, 28, 30, 35, 40, 45, 50, 60, 70, 80, 90, 100 or more). The fragment may comprise at least one T-cell or, preferably, a B-cell epitope of the sequence. T- and B-cell epitopes can be identified empirically (e.g. using PEPSCAN [14,15] or similar methods), or they can be predicted (e.g. using the Jameson-Wolf antigenic index [16], matrix-based approaches [17], TEPITOPE [18], neural networks [19], OptiMer & EpiMer [20,21], ADEPT [22], Tsites [23], hydrophilicity [24], antigenic index [25] or the methods disclosed in reference 26, etc.).

Other preferred fragments are (a) the N-terminal signal peptides of the polypeptides of the invention, (b) the polypeptides, but without their N-terminal signal peptides, (c) the polypeptides, but without their N-terminal amino acid residue. Thus the invention further provides truncated sequences of the polypeptides of the invention. The sequences may be truncated at the N-terminus and/or the C-terminus. Truncation may involve a single amino acid or a longer sequence. A truncated sequence preferably retains at least one epitope of the pre-truncation sequence. For example, the invention provides truncated sequence, SEQ ID NO: 168 which is amino acids 21-470 of SEQ ID NO:56.

Other preferred fragments are those that are common to a polypeptide of the invention and to a polypeptide identified in any of references 5, 6, 8, 10 and 11.

Other preferred fragments are those that are common to a polypeptide of the invention and to a polypeptide identified in reference 27, reference 28, U.S. Provisional Application No. 60/654,632 (filed Feb. 18, 2005; priority application for refs 27 & 28) or U.S. Provisional Application No. 60/712,720 (filed Aug. 29, 2005; priority application for ref. 28).

The invention also provides polypeptides comprising amino acid sequences that have sequence identity to, and comprise fragments of, the amino acid sequences disclosed in the examples. Depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more), and the fragments should comprise at least n consecutive amino acids from the sequences and, depending on the particular sequence, 11 is 7 or more (e.g. 8, 10, 12, 14, 16, 18, 20, 22, 24, 26, 28, 30, 35, 40, 45, 50, 60, 70, 80, 90, 100 or more).

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [29,30]. Solid-phase peptide synthesis is particularly preferred, such as methods based on tBoc or Fmoc [31] chemistry. Enzymatic synthesis [32] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [33]. Where D-amino acids are included, however, it is preferred to use chemical synthesis. Polypeptides of the invention may have covalent modifications at the C-terminus and/or N-terminus.

Polypeptides of the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides of the invention are preferably provided in purified or substantially purified form i.e. substantially free from other polypeptides (e.g. free from naturally-occurring polypeptides), particularly from other ExPEC or host cell polypeptides, and are generally at least about 50% pure (by weight), and usually at least about 90% pure i.e. less than about 50%, and more preferably less than about 10% (e.g. 5% or less) of a composition is made up of other expressed polypeptides. Polypeptides of the invention are preferably ExPEC polypeptides.

Polypeptides of the invention may be attached to a solid support. Polypeptides of the invention may comprise a detectable label (e.g. a radioactive or fluorescent label, or a biotin label).

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides of the invention can be naturally or non-naturally glycosylated (i.e. the polypeptide has a glycosylation pattern that differs from the glycosylation pattern found in the corresponding naturally occurring polypeptide).

Polypeptides of the invention may be at least 40 amino acids long (e.g. at least 40, 50, 60, 70, 80, 90, 100, 120, 140, 160, 180, 200, 220, 240, 260, 280, 300, 350, 400, 450, 500 or more). Polypeptides of the invention may be shorter than 500 amino acids (e.g. no longer than 40, 50, 60, 70, 80, 90, 100, 120, 140, 160, 180, 200, 220, 240, 260, 280, 300, 350, 400 or 450 amino acids).

The invention provides polypeptides comprising a sequence —X—Y— or —Y—X—, wherein: —X— is an amino acid sequence as defined above and —Y— is not a sequence as defined above i.e. the invention provides fusion proteins. Where the N-terminus codon of a polypeptide-coding sequence is not ATG then that codon will be translated as the standard amino acid for that codon rather than as a Met, which occurs when the codon is translated as a start codon.

The invention provides a process for producing polypeptides of the invention, comprising culturing a host cell of to the invention under conditions which induce polypeptide expression.

The invention provides a process for producing a polypeptide of the invention, wherein the polypeptide is synthesised in part or in whole using chemical means.

The invention provides a composition comprising two or more polypeptides of the invention.

The invention also provides a hybrid polypeptide represented by the formula NH₂-A-[-X-L-]ₙ—B—COOH, wherein X is a polypeptide of the invention as defined above, L is an optional linker amino acid sequence, A is an optional N-terminal amino acid sequence, B is an optional C-terminal amino acid sequence, and n is an integer greater than 1. The value of n is between 2 and x, and the value of x is typically 3, 4, 5, 6, 7, 8, 9 or 10. Preferably n is 2, 3 or 4; it is more preferably 2 or 3; most preferably, n=2. For each n instances, —X— may be the same or different. For each n instances of [-X-L-], linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s)-L- will typically be short (e.g. 20 or fewer amino acids i.e. 19, 18, 17, 16, 15, 14, 13, 12, 11, 10, 9, 8, 7, 6, 5, 4, 3, 2, 1). Examples include short peptide sequences which facilitate cloning, poly-glycine linkers (i.e. Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art.-A- and -B- are optional sequences which will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct polypeptide trafficking, or short peptide sequences which facilitate cloning or purification (e.g. histidine tags i.e. Hisₙ where n=3, 4, 5, 6, 7, 8, 9, 10 or more). Other suitable N-terminal and C-terminal amino acid sequences will be apparent to those skilled in the art.

Various tests can be used to assess the in vivo immunogenicity of polypeptides of the invention. For example, polypeptides can be expressed recombinantly and used to screen patient sera by immunoblot. A positive reaction between the polypeptide and patient serum indicates that the patient has previously mounted an immune response to the protein in question i.e. the protein is an immunogen. This method can also be used to identify immunodominant proteins.

### Antibodies

The invention provides antibodies that bind to polypeptides of the invention. These may be polyclonal or monoclonal and may be produced by any suitable means (e.g. by recombinant expression). To increase compatibility with the human immune system, the antibodies may be chimeric or humanised [e.g. refs. 34 & 35], or fully human antibodies may be used. The antibodies may include a detectable label (e.g. for diagnostic assays). Antibodies of the invention may be attached to a solid support. Antibodies of the invention are preferably neutralising antibodies.

Monoclonal antibodies are particularly useful in identification and purification of the individual polypeptides against which they are directed. Monoclonal antibodies of the invention may also be employed as reagents in immunoassays, radioimmunoassays (RIA) or enzyme-linked immunosorbent assays (ELISA), etc. In these applications, the antibodies can be labelled with an analytically-detectable reagent such as a radioisotope, a fluorescent molecule or an enzyme. The monoclonal antibodies produced by the above method may also be used for the molecular identification and characterization (epitope mapping) of polypeptides of the invention.

Antibodies of the invention are preferably specific to ExPEC strains of E. coli, i.e. they bind preferentially to ExPEC E. coli relative to other bacteria (e.g. relative to non-ExPEC E. coli and relative to non-E. coli bacteria). More preferably, the antibodies are specific to UPEC strains i.e. they bind preferentially to UPEC bacteria relative to other bacteria, including other ExPEC E. coli.

Antibodies of the invention are preferably provided in purified or substantially purified form. Typically, the antibody will be present in a composition that is substantially free of other polypeptides e.g. where less than 90% (by weight), usually less than 60% and more usually less than 50% of the composition is made up of other polypeptides.

Antibodies of the invention can be of any isotype (e.g. IgA, IgG, IgM i.e. an α, γ or μ heavy chain), but will generally be IgG. Within the IgG isotype, antibodies may be IgG1, IgG2, IgG3 or IgG4 subclass. Antibodies of the invention may have a x or a X, light chain.

Antibodies of the invention can take various forms, including whole antibodies, antibody fragments such as F(ab′)₂ and F(ab) fragments, Fv fragments (non-covalent heterodimers), single-chain antibodies such as single chain Fv molecules (scFv), minibodies, oligobodies, etc. The term “antibody” does not imply any particular origin, and includes antibodies obtained through non-conventional processes, such as phage display.

The invention provides a process for detecting polypeptides of the invention, comprising the steps of: (a) contacting an antibody of the invention with a biological sample under conditions suitable for the formation of an antibody-antigen complexes; and (b) detecting said complexes.

The invention provides a process for detecting antibodies of the invention, comprising the steps of: (a) contacting a polypeptide of the invention with a biological sample (e.g. a blood or serum sample) under conditions suitable for the formation of an antibody-antigen complexes; and (b) detecting said complexes.

Preferred antibodies bind to a polypeptide of the invention with substantially greater affinity than antibodies known in the art. Preferably, the affinity is at least 1.5-fold, 2-fold, 5-fold 10-fold, 100-fold, 10³-fold, 10⁴-fold, 10⁵-fold, 10⁶-fold etc. stronger than antibodies known in the art.

### Nucleic Acids

The invention also provides nucleic acid comprising a nucleotide sequence encoding the polypeptides of the invention. The invention also provides nucleic acid comprising nucleotide sequences having sequence identity to such nucleotide sequences. Identity between sequences is preferably determined by the Smith-Waterman homology search algorithm as described above. Such nucleic acids include those using alternative codons to encode the same amino acid.

The invention also provides nucleic acid which can hybridize to these nucleic acids. Hybridization reactions can be performed under conditions of different “stringency”. Conditions that increase stringency of a hybridization reaction of widely known and published in the art [e.g. page 7.52 of reference 297]. Examples of relevant conditions include (in order of increasing stringency): incubation temperatures of 25° C., 37° C., 50° C., 55° C. and 68° C.; buffer concentrations of 10×SSC, 6 x SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art [e.g. see refs 36, 37, 297, 299, etc.].

In some embodiments, nucleic acid of the invention hybridizes to a target under low stringency conditions; in other embodiments it hybridizes under intermediate stringency conditions; in preferred embodiments, it hybridizes under high stringency conditions. An exemplary set of low stringency hybridization conditions is 50° C. and 10×SSC. An exemplary set of intermediate stringency hybridization conditions is 55° C. and 1×SSC. An exemplary set of high stringency hybridization conditions is 68° C. and 0.1×SSC.

Nucleic acid comprising fragments of these sequences are also provided. These should comprise at least n consecutive nucleotides from the sequences and, depending on the particular sequence, n is 10 or more (e.g. 12, 14, 15, 18, 20, 25, 30, 35, 40, 50, 60, 70, 80, 90, 100, 150, 200 or more). Preferred fragments are those that are common to a nucleic acid sequence of the invention and to a nucleic acid sequence identified in any of references 5, 6, 8, 10 and 11.

The invention provides nucleic acid of formula 5′—X—Y—Z—3′, wherein: —X— is a nucleotide sequence consisting of x nucleotides; —Z— is a nucleotide sequence consisting of z nucleotides; —Y— is a nucleotide sequence consisting of either (a) a fragment of a nucleic acid sequence encoding one of SEQ ID NOS: 1 to 168 or (b) the complement of (a); and said nucleic acid 5′—X—Y—Z—3′ is neither (i) a fragment of either a nucleic acid sequence encoding one of SEQ ID NOS: 1 to 168 nor (ii) the complement of (i). The —X—. and/or —Z— moieties may comprise a promoter sequence (or its complement).

The invention includes nucleic acid comprising sequences complementary to these sequences (e.g. for antisense or probing, or for use as primers).

Nucleic acids of the invention can be used in hybridisation reactions (e.g. Northern or Southern blots, or in nucleic acid microarrays or ‘gene chips’) and amplification reactions (e.g. PCR, SDA, SSSR, LCR, TMA, NASBA, etc.) and other nucleic acid techniques.

Nucleic acid according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other ExPEC or host cell nucleic acids, generally being at least about 50% pure (by weight), and usually at least about 90% pure. Nucleic acids of the invention are preferably ExPEC nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids of the invention comprise sequences, but they may also comprise non-ExPEC sequences (e.g. in nucleic acids of formula 5′—X—Y—Z—3′, as defined above). This is particularly useful for primers, which may thus comprise a first sequence complementary to a nucleic acid target and a second sequence which is not complementary to the nucleic acid target. Any such non-complementary sequences in the primer are preferably 5′ to the complementary sequences. Typical non-complementary sequences comprise restriction sites or promoter sequences.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T). The terms also imply a direction—the complement of 5′-ACAGT-3′ is 5′-ACTGT-3′ rather than 5′-TGTCA-3′.

Nucleic acids of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

The invention also provides a kit comprising primers (e.g. PCR primers) for amplifying a template sequence contained within an ExPEC nucleic acid sequence, the kit comprising a first primer and a second primer, wherein the first primer is substantially complementary to said template sequence and the second primer is substantially complementary to a complement of said template sequence, wherein the parts of said primers which have substantial complementarity define the termini of the template sequence to be amplified. The first primer and/or the second primer may include a detectable label (e.g. a fluorescent label).

The invention also provides a kit comprising first and second single-stranded oligonucleotides which allow amplification of a ExPEC template nucleic acid sequence contained in a single- or double-stranded nucleic acid (or mixture thereof), wherein: (a) the first oligonucleotide comprises a primer sequence which is substantially complementary to said template nucleic acid sequence; (b) the second oligonucleotide comprises a primer sequence which is substantially complementary to the complement of said template nucleic acid sequence; (c) the first oligonucleotide and/or the second oligonucleotide comprise(s) sequence which is not complementary to said template nucleic acid; and (d) said primer sequences define the termini of the template sequence to be amplified. The non-complementary sequence(s) of feature (c) are preferably upstream of (i.e. 5′ to) the primer sequences. One or both of these (c) sequences may comprise a restriction site [e.g. ref. 38] or a promoter sequence [e.g. 39]. The first oligonucleotide and/or the second oligonucleotide may include a detectable label (e.g. a fluorescent label).

The invention provides a process for detecting nucleic acid of the invention, comprising: (a) contacting a nucleic probe according to the invention with a biological sample under hybridising conditions to form duplexes; and (b) detecting said duplexes.

The invention provides a process for detecting in a biological sample (e.g. blood), comprising contacting nucleic acid according to the invention with the biological sample under hybridising conditions. The process may involve nucleic acid amplification (e.g. PCR, SDA, SSSR, LCR, TMA, NASBA, etc.) or hybridisation (e.g. microarrays, blots, hybridisation with a probe in solution etc.). PCR detection of ExPEC in clinical samples has been reported [e.g. see ref. 40]. Clinical assays based on nucleic acid are described in general in ref. 41.

The invention provides a process for preparing a fragment of a target sequence, wherein the fragment is prepared by extension of a nucleic acid primer. The target sequence and/or the primer are nucleic acids of the invention. The primer extension reaction may involve nucleic acid amplification (e.g. PCR, SDA, SSSR, LCR, TMA, NASBA, etc.).

Nucleic acid amplification according to the invention may be quantitative and/or real-time.

For certain embodiments of the invention, nucleic acids are preferably at least 7 nucleotides in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 nucleotides or longer).

For certain embodiments of the invention, nucleic acids are preferably at most 500 nucleotides in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 nucleotides or shorter).

Primers and probes of the invention, and other nucleic acids used for hybridization, are preferably between 10 and 30 nucleotides in length (e.g. 10, 11, 12, 13, 14, 15, 16, 17, 18, 19, 20, 21, 22, 23, 24, 25, 26, 27, 28, 29, or 30 nucleotides).

### Vesicles

Reference 42 describes the preparation of vesicles from a uropathogenic (UPEC) strain by the knockout of mltA (a murein lytic transglycosylase) or one or more of the components of the E. coli Tol-Pal complex [43], such as tolA, tolQ, tolB, pal and/or tolR. These vesicles can be improved by making one or more further genetic changes to the chromosome of the bacterium or through insertion of episomal elements (e.g. expression vectors) in order to increase the amount of and/or immunoaccessibility of protective antigens on the surface the vesicles.

One way of obtaining such improvements is to up-regulate the expression of the polypeptides of the invention. Many different genetic strategies for increasing the expression of a target protein are well-known in the art and can be distinguished into two broad categories: one relying on modifications of the chromosome (e.g. replacement of the wild-type promoter with a stronger promoter, inactivation of natural repressor genes, etc.) to increase expression of an endogenous gene, and the other based on recombinant expression by episomal elements (e.g. high-copy number plasmids, vectors harboring an engineered target gene, etc.) or integration of a exogenous gene in the chromosome. Practical examples for each of these approaches can be found in references 44 to 50.

Another way of increasing vesicle immunogenicity and selectivity is to down-regulate the expression of immunodominant non-protective antigens or to down-regulate proteins that are homologous to proteins found in commensal strains. Further improvements can be achieved by detoxification of the Lipid A moiety of LPS. Similar changes have been previously described to produce improved vesicles from other Gram-negative pathogens (see for example references 51 & 52).

All the above strategies can be used either alone or in combination to obtain improved vesicles for use in immunogenic compositions. The invention provides a pathogenic Escherichia coli bacterium (particularly a UPEC) having a knockout of mltA and/or of a component of its Tol-Pal complex, and one or more of: (i) a chromosomal gene encoding a polypeptide of the invention under the control of a promoter that provides higher expression levels of the polypeptide than the promoter that is naturally associated with the gene encoding the polypeptide; or (ii) an autonomously-replicating extrachromosomal element encoding a polypeptide of the invention, and optionally also (iii) a genetic modification to reduce the toxicity of the Lipid A moiety of E. coli LPS relative to wild-type LPS.

The invention also provides vesicles obtainable by culturing such a bacterium, such as the vesicles that, during culture of the bacterium, are released into the culture medium.

In a particular aspect, the invention provides immunogenic compositions comprising one or more outer membrane vesicles (OMVs) expressing one or more polypeptides of the invention. In a particular embodiment, the invention provides an immunogenic composition comprising one or more OMVs expressing one or more polypeptides comprising: (a) an amino acid sequence selected from the group consisting of SEQ ID NOs 1 to 168; (b) an amino acid sequence having at least 80% sequence identity to an amino acid sequence of (a); (c) an amino acid sequence which is a fragment of at least 10 consecutive amino acids from an amino acid sequence of (a); or (d) an amino acid sequence having at least 80% sequence identity to an amino acid sequence of (a) and including a fragment of at least 10 consecutive amino acids from an amino acid sequence of (a). In a further embodiment, the immunogenic composition comprises a polypeptide comprising a fragment which comprises at least one B-cell epitope of an amino acid sequence selected from the group consisting of SEQ ID NOs 1-168.

### Pharmaceutical Compositions

The invention provides compositions comprising: (a) polypeptide, antibody, vesicles and/or nucleic acid of the invention; and (b) a pharmaceutically acceptable carrier. These compositions may be suitable as immunogenic compositions, for instance, or as diagnostic reagents, or as vaccines. Vaccines according to the invention may either be prophylactic (i.e. to prevent infection) or therapeutic (i.e. to treat infection), but will typically be prophylactic.

A ‘pharmaceutically acceptable carrier’ includes any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in ref. 294.

Compositions of the invention may include an antimicrobial, particularly if packaged in a multiple dose format.

Compositions of the invention may comprise detergent e.g. a Tween (polysorbate), such as Tween 80. Detergents are generally present at low levels e.g. <0.01%.

Compositions of the invention may include sodium salts (e.g. sodium chloride) to give tonicity. A concentration of 10±2 mg/ml NaCl is typical.

Compositions of the invention will generally include a buffer. A phosphate buffer is typical.

Compositions of the invention may comprise a sugar alcohol (e.g. mannitol) or a disaccharide (e.g. sucrose or trehalose) e.g. at around 15-30 mg/ml (e.g. 25 mg/ml), particularly if they are to be lyophilised or if they include material which has been reconstituted from lyophilised material. The pH of a composition for lyophilisation may be adjusted to around 6.1 prior to lyophilisation.

Polypeptides of the invention may be administered in conjunction with other immunoregulatory agents. In particular, compositions will usually include a vaccine adjuvant. The adjuvant may be selected from one or more of the group consisting of a TH1 adjuvant and TH2 adjuvant, further discussed below. Adjuvants which may be used in compositions of the invention include, but are not limited to:

### A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref. 53], or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. Mineral containing compositions may also be formulated as a particle of metal salt [54].

A typical aluminium phosphate adjuvant is amorphous aluminium hydroxyphosphate with PO₄/Al molar ratio between 0.84 and 0.92, included at 0.6 mg Al³⁺/m-1. Adsorption with a low dose of aluminium phosphate may be used e.g. between 50 and 100 μg Al³⁺ per conjugate per dose. Where an aluminium phosphate it used and it is desired not to adsorb an antigen to the adjuvant, this is favoured by including free phosphate ions in solution (e.g. by the use of a phosphate buffer).

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

The invention can use a mixture of both an aluminium hydroxide and an aluminium phosphate. In this case there may be more aluminium phosphate than hydroxide e.g. a weight ratio of at least 2:1 e.g. ≧5:1, ≧6:1, ≧7:1, ≧8:1, ≧9:1, etc.

Aluminum salts may be included in vaccines of the invention such that the dose of Al³⁺ is between 0.2 and 1.0 mg per dose.

### B. Oil Emulsions

Oil emulsion compositions suitable for use as adjuvants in the invention include squalene-water emulsions, such as MF59 (5% Squalene, 0.5% Tween 80, and 0.5% Span 85, formulated into submicron particles using a microfluidizer) [Chapter 10 of ref. 53; see also refs. 55-57, chapter 12 of ref. 58]. MF59 is used as the adjuvant in the FLUAD™ influenza virus trivalent subunit vaccine. The emulsion advantageously includes citrate ions e.g. 10 mM sodium citrate buffer.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80 (polyoxyelthylenesorbitan monooleate), and/or 0.25-1.0% Span 85 (sorbitan trioleate), and, optionally, N-acetylmuramyl-L-alanyl-D-isogluatminyl-L-alanine-2-(1′-2′-dipalmitoyl-sn-glycero-3-hydroxyphosphosphoryloxy)-ethylamine (MTP-PE). Submicron oil-in-water emulsions, methods of making the same and immunostimulating agents, such as muramyl peptides, for use in the compositions, are described in detail in references 55 & 59-60.

An emulsion of squalene, a tocopherol, and Tween 80 can be used. The emulsion may include phosphate buffered saline. It may also include Span 85 (e.g. at 1%) and/or lecithin. These emulsions may have from 2 to 10% squalene, from 2 to 10% tocopherol and from 0.3 to 3% Tween 80, and the weight ratio of squalene:tocopherol is preferably ≦1 as this provides a more stable emulsion. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm.

An emulsion of squalene, a tocopherol, and a Triton detergent (e.g. Triton X-100) can be used.

An emulsion of squalane, polysorbate 80 and poloxamer 401 (“Pluronic™ L121”) can be used. The emulsion can be formulated in phosphate buffered saline, pH 7.4. This emulsion is a useful delivery vehicle for muramyl dipeptides, and has been used with threonyl-MDP in the “SAF-1” adjuvant [61] (0.05-1% Thr-MDP, 5% squalane, 2.5% Pluronic L121 and 0.2% polysorbate 80). It can also be used without the Thr-MDP, as in the “AF” adjuvant [62] (5% squalane, 1.25% Pluronic L121 and 0.2% polysorbate 80). Microfluidisation is preferred.

Complete Freund's adjuvant (CFA) and incomplete Freund's adjuvant (IFA) may also be used as adjuvants in the invention.

### C. Saponin Formulations [Chapter 22 of Ref 53]

Saponin formulations may also be used as adjuvants in the invention. Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 63. Saponin formulations may also comprise a sterol, such as cholesterol [64].

Combinations of saponins and cholesterols can be used to form unique particles called immunostimulating complexs (ISCOMs) [chapter 23 of ref. 53]. ISCOMs typically also include a phospholipid such as phosphatidylethanolamine or phosphatidylcholine. Any known saponin can be used in ISCOMs. Preferably, the ISCOM includes one or more of QuilA, QHA and QHC. ISCOMs are further described in refs. 64-66. Optionally, the ISCOMS may be devoid of additional detergent(s) [67].

A review of the development of saponin based adjuvants can be found in refs. 68 & 69.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, QB-phage (such as coat proteins), GA-phage, fr-phage, AP205 phage, and Ty (such as retrotransposon Ty protein p1). VLPs are discussed further in refs. 70-75. Virosomes are discussed further in, for example, ref. 76

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 77. Such “small particles” of 3dMPL are small enough to be sterile filtered through a 0.22 μm membrane [77]. Other non-toxic LPS derivatives include monophosphoryl lipid A mimics, such as aminoalkyl glucosaminide phosphate derivatives e.g. RC-529 [78,79].

Lipid A derivatives include derivatives of lipid A from Escherichia coli such as OM-174. OM-174 is described for example in refs. 80 & 81.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 82, 83 and 84 disclose possible analog substitutions e.g. replacement of guanosine with 2′-deoxy-7-deazaguanosine. The adjuvant effect of CpG oligonucleotides is further discussed in refs. 85-90.

The CpG sequence may be directed to TLR9, such as the motif GTCGTT or TTCGTT [91]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 92-94. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 91 & 95-97.

Other immunostimulatory oligonucleotides include a double-stranded RNA, or an oligonucleotide containing a palindromic sequence, or an oligonucleotide containing a poly(dG) sequence.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref 98 and as parenteral adjuvants in ref. 99. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivaties thereof, particularly LT-K63 and LT-R72, as adjuvants can be found in refs. 100-107. Numerical reference for amino acid substitutions is preferably based on the alignments of the A and B subunits of ADP-ribosylating toxins set forth in ref 108.

Compounds of formula I, II or III, or salts thereof, can also be used as adjuvants:

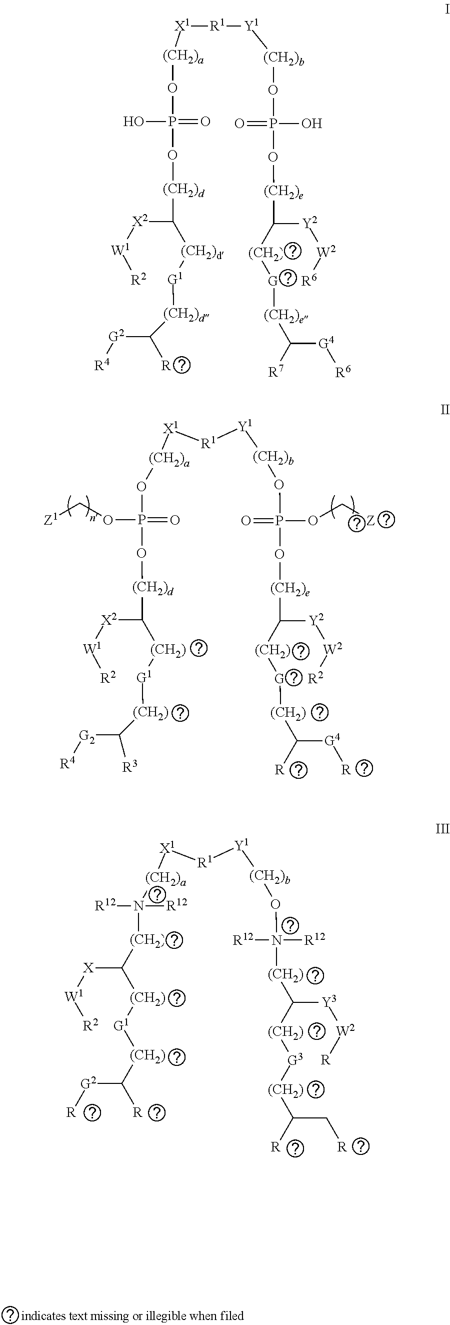

as defined in reference 109, such as ‘ER 803058’, ‘ER 803732’, ‘ER 804053’, ER 804058′, ‘ER 804059’, ‘ER 804442’, ER 804680′, ‘ER 804764’, ER 803022 or ‘ER 804057’ e.g.:

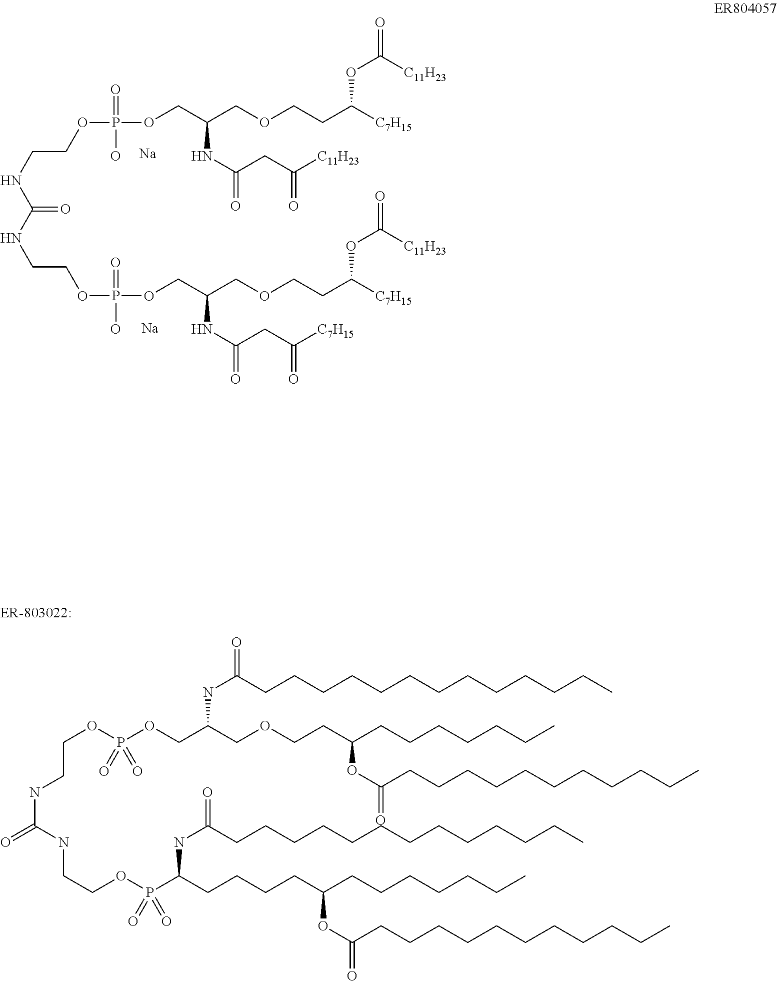

### F. Human Immunomodulators

Human immunomodulators suitable for use as adjuvants in the invention include cytokines, such as interleukins (e.g. IL-1, IL-2, IL-4, IL-5, IL-6, IL-7, IL-12 [110], etc.) [111], interferons (e.g. interferon-γ), macrophage colony stimulating factor, tumor necrosis factor and macrophage inflammatory protein-1alpha (MIP-1alpha) and MIP-1beta [112].

### G. Bioadhesives and Mucoadhesives

Bioadhesives and mucoadhesives may also be used as adjuvants in the invention. Suitable bioadhesives include esterified hyaluronic acid microspheres [113] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [114].

### H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### I. Liposomes (Chapters 13 & 14 of ref 53)

Examples of liposome formulations suitable for use as adjuvants are described in refs. 115-117.

### J. Polyoxyethylene Ether and Polyoxyethylene Ester Formulations

Adjuvants suitable for use in the invention include polyoxyethylene ethers and polyoxyethylene esters [118]. Such formulations further include polyoxyethylene sorbitan ester surfactants in combination with an octoxynol [119] as well as polyoxyethylene alkyl ethers or ester surfactants in combination with at least one additional non-ionic surfactant such as an octoxynol [120]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### K Phosphazenes E.G. PCPP

Phosphazene adjuvants include poly[di(carboxylatophenoxy)phosphazene] (“PCPP”) as described, for example, in refs. 121 and 122.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M Imidazoquinoline Compounds.

Imidazoquinoline adjuvants include Imiquimod (“R-837”) [123,124], Resiquimod (“R-848”) [125], and their analogs; and salts thereof (e.g. the hydrochloride salts). Further details about immunostimulatory imidazoquinolines can be found in references 126 to 130.

### N. Thiosemicarbazone Compounds.

Examples of thiosemicarbazone compounds, as well as methods of formulating, manufacturing, and screening for compounds all suitable for use as adjuvants in the invention include those described in ref. 131. The thiosemicarbazones are particularly effective in the stimulation of human peripheral blood mononuclear cells for the production of cytokines, such as TNF-α.

### O. Tryptanthrin Compounds.

Examples of tryptanthrin compounds, as well as methods of formulating, manufacturing, and screening for compounds all suitable for use as adjuvants in the invention include those described in ref. 132. The tryptanthrin compounds are particularly effective in the stimulation of human peripheral blood mononuclear cells for the production of cytokines, such as TNF-α.

### P. Nucleoside Analogs

Various nucleoside analogs can be used as adjuvants, such as (a) Isatorabine (ANA-245; 7-thia-8-oxoguanosine):

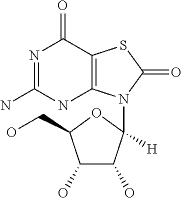 [SMILES: Nc1nc(=O)c2sc(=O)n([C@@H]3O[C@H](CO)[C@@H](O)[C@H]3O)c2[nH]1]

and prodrugs thereof; (b) ANA975; (c) ANA-025-1; (d) ANA380; (e) the compounds disclosed in references 133 to 135; (f) a compound having the formula:

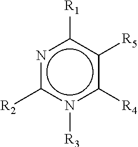

wherein:
R₁ and R₂ are each independently H, halo, —NRₐR_{b}, —OH, C₁₋₆ alkoxy, substituted C₁₋₆ alkoxy, heterocyclyl, substituted heterocyclyl, C₆₋₁₀ aryl, substituted C₆₋₁₀ aryl, C₁₋₆ alkyl, or substituted C₁₋₆ alkyl;
R₃ is absent, H, C₁₋₆ alkyl, substituted C₁₋₆ alkyl, C₆₋₁₀ aryl, substituted C₆₋₁₀ aryl, heterocyclyl, or substituted heterocyclyl;
R₄ and R₅ are each independently H, halo, heterocyclyl, substituted heterocyclyl, —C(O)—R_{d}, C₁₋₆ alkyl, substituted C₁₋₆ alkyl, or bound together to form a 5 membered ring as in R₄₋₅:

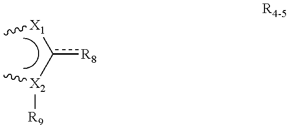

the binding being achieved at the bonds indicated by a ˜
X₁ and X₂ are each independently N, C, O, or S;
R₈ is H, halo, —OH, C₁₋₆ alkyl, C₂₋₆ alkenyl, C₂₋₆ alkynyl, —OH, —NRₐR_{b}, —(CH₂)ₙ—O—R_{c}, —O—(C₁₋₆ alkyl), —S(O)ₚRₑ, or —C(O)—R_{d};
R₉ is H, C₁₋₆ alkyl, substituted C₁₋₆ alkyl, heterocyclyl, substituted heterocyclyl or R₉ₐ, wherein R₉ₐ is:

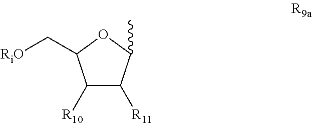

the binding being achieved at the bond indicated by a
R₁₀ and R₁₁ are each independently H, halo, C₁₋₆ alkoxy, substituted C₁₋₆ alkoxy, —NRₐR_{b}, or —OH;
each Rₐ and R_{b} is independently H, C₁₋₆ alkyl, substituted C₁₋₆ alkyl, —C(O)R_{d}, C₆₋₁₀ aryl;
each R_{c} is independently H, phosphate, diphosphate, triphosphate, C₁₋₆ alkyl, or substituted C₁₋₆ alkyl;
each R_{d} is independently H, halo, C₁₋₆ alkyl, substituted C₁₋₆ alkyl, C₁₋₆ alkoxy, substituted C₁₋₆ alkoxy, —NH₂, —NH(C₁₋₆ alkyl), —NH(substituted C₁₋₆ alkyl), —N(C₁₋₆ alkyl)₂, —N(substituted C₁₋₆ alkyl)₂, C₆₋₁₀ aryl, or heterocyclyl;
each Rₑ is independently H, C₁₋₆ alkyl, substituted C₁₋₆ alkyl, C₆₋₁₀ aryl, substituted C₆₋₁₀ aryl, heterocyclyl, or substituted heterocyclyl;
each R_{f} is independently H, C₁₋₆ alkyl, substituted C₁₋₆ alkyl, —C(O)R_{d}, phosphate, diphosphate, or triphosphate;
each n is independently 0, 1, 2, or 3;
each p is independently 0, 1, or 2; or
   or (g) a pharmaceutically acceptable salt of any of (a) to (f), a tautomer of any of (a) to (f), or a pharmaceutically acceptable salt of the tautomer.

### Q. Lipids Linked to a Phosphate-Containing Acyclic Backbone

Adjuvants containing lipids linked to a phosphate-containing acyclic backbone include the TLR4 antagonist E5564 [136,137]:

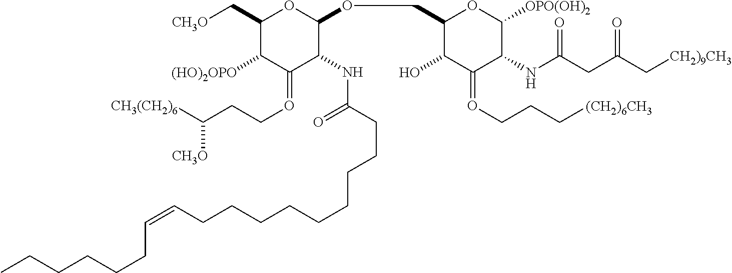

### R. Small Molecule Immunopotentiators (SMIPs)

SMIPs include:
N2-methyl-1-(2-methylpropyl)-1H-imidazo[4,5-c]quinoline-2,4-diamine;
N2,N2-dimethyl-1-(2-methylpropyl)-1H-imidazo[4,5-c]quinoline-2,4-diamine;
N2-ethyl-N2-methyl-1-(2-methylpropyl)-1H-imidazo[4,5-c]quinoline-2,4-diamine;
N2-methyl-1-(2-methylpropyl)-N2-propyl-1H-imidazo[4,5-c]quinoline-2,4-diamine;
1-(2-methylpropyl)-N2-propyl-1H-imidazo[4,5-c]quinoline-2,4-diamine;
N2-butyl-1-(2-methylpropyl)-1H-imidazo[4,5-c]quinoline-2,4-diamine;
N2-butyl-N2-methyl-1-(2-methylpropyl)-1H-imidazo[4,5-c]quinoline-2,4-diamine;
N2-methyl-1-(2-methylpropyl)-N2-pentyl-1H-imidazo[4,5-c]quinoline-2,4-diamine;
N2-methyl-1-(2-methylpropyl)-N2-prop-2-enyl-1H-imidazo[4,5-c]quinoline-2,4-diamine;
1-(2-methylpropyl)-2-[(phenylmethyl)thio]-1H-imidazo[4,5-c]quinolin-4-amine;
1-(2-methylpropyl)-2-(propylthio)-1H-imidazo[4,5-c]quinolin-4-amine
2-[[4-amino-1-(2-methylpropyl)-1H-imidazo[4,5-c]quinolin-2-yl](methyl)amino]ethanol;
2-[[4-amino-1-(2-methylpropyl)-1H-imidazo[4,5-c]quinolin-2-yl](methyl)amino]ethyl acetate;
4-amino-1-(2-methylpropyl)-1,3-dihydro-2H-imidazo[4,5-c]quinolin-2-one;
N2-butyl-1-(2-methylpropyl)-N4,N4-bis(phenylmethyl)-1H-imidazo[4,5-c]quinoline-2,4-diamine;
N2-butyl-N2-methyl-1-(2-methylpropyl)-N4,N4-bis(phenylmethyl)-1H-imidazo[4,5-c]quinoline-2,4-diamine;
N2-methyl-1-(2-methylpropyl)-N4,N4-bis(phenylmethyl)-1H-imidazo[4,5-c]quinoline-2,4-diamine;
N2,N2-dimethyl-1-(2-methylpropyl)-N4,N4-bis(phenylmethyl)-1H-imidazo[4,5-c]quinoline-2,4-diamine;
1-{4-amino-2-[methyl(propyl)amino]-1H-imidazo[4,5-c]quinolin-1-yl}-2-methylpropan-2-ol;
1-[4-amino-2-(propylamino)-1H-imidazo[4,5-c]quinolin-1-yl]-2-methylpropan-2-ol;
N4,N4-dibenzyl-1-(2-methoxy-2-methylpropyl)-N2-propyl-1H-imidazo[4,5-c]quinoline-2,4-diamine.

### S. Proteosomes

One adjuvant is an outer membrane protein proteosome preparation prepared from a first Gram-negative bacterium in combination with a liposaccharide preparation derived from a second Gram-negative bacterium, wherein the outer membrane protein proteosome and liposaccharide preparations form a stable non-covalent adjuvant complex. Such complexes include “IVX-908”, a complex comprised of Neisseria meningitidis outer membrane and lipopolysaccharides. They have been used as adjuvants for influenza vaccines [138].

### T. Other Adjuvants

Other substances that act as immunostimulating agents are disclosed in references 53 and 58. Further useful adjuvant substances include:
Methyl inosine 5′-monophosphate (“MIMP”) [139].
A polyhydroxlated pyrrolizidine compound [140], such as one having formula:

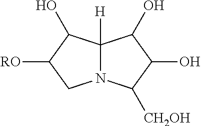

where R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. Examples include, but are not limited to: casuarine, casuarine-6-α-D-glucopyranose, 3-epi-casuarine, 7-epi-casuarine, 3,7-diepi-casuarine, etc.
A gamma inulin [141] or derivative thereof, such as algammulin.
Compounds disclosed in reference 142.
Compounds disclosed in reference 143, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds [144,145], Hydrapthalamide compounds, Benzophenone compounds, Isoxazole compounds, Sterol compounds, Quinazilinone compounds, Pyrrole compounds [146], Anthraquinone compounds, Quinoxaline compounds, Triazine compounds, Pyrazalopyrimidine compounds, and Benzazole compounds [147].
Loxoribine (7-allyl-8-oxoguanosine) [148].
A formulation of a cationic lipid and a (usually neutral) co-lipid, such as aminopropyl-dimethyl-myristoleyloxy-propanaminium bromide-diphytanoylphosphatidyl-ethanolamine (“Vaxfectin™”) or aminopropyl-dimethyl-bis-dodecyloxy-propanaminium bromide-dioleoylphosphatidyl-ethanolamine (“GAP-DLRIE:DOPE”). Formulations containing (±)—N-(3-aminopropyl)-N,N-dimethyl-2,3-bis(syn-9-tetradeceneyloxy)-1-propanaminium salts are preferred [149].

The invention may also comprise combinations of one or more of the adjuvants identified above. For example, the following combinations may be used as adjuvant compositions in the invention: (1) a saponin and an oil-in-water emulsion [150]; (2) a saponin (e.g. QS21)+a non-toxic LPS derivative (e.g. 3dMPL) [151]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [152]; (5) combinations of 3dMPL with, for example, QS21 and/or oil-in-water emulsions [153]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (8) one or more mineral salts (such as an aluminum salt)+a non-toxic derivative of LPS (such as 3dMPL); and (9) one or more mineral salts (such as an aluminum salt)+an immunostimulatory oligonucleotide (such as a nucleotide sequence including a CpG motif).

The compositions of the invention will preferably elicit both a cell mediated immune response as well as a humoral immune response in order to effectively address a uropathogenic infection. This immune response will preferably induce long lasting (e.g. neutralising) antibodies and a cell mediated immunity that can quickly respond upon exposure to UPEC-associated antigens.

Two types of T cells, CD4 and CD8 cells, are generally thought necessary to initiate and/or enhance cell mediated immunity and humoral immunity. CD8 T cells can express a CD8 co-receptor and are commonly referred to as cytotoxic T lymphocytes (CTLs). CD8 T cells are able to recognized or interact with antigens displayed on MHC Class 1 molecules. CD4 T cells can express a CD4 co-receptor and are commonly referred to as T helper cells. CD4 T cells are able to recognize antigenic peptides bound to MHC class I1 molecules. Upon interaction with a MHC class I1 molecule, the CD4 cells can secrete factors such as cytokines. These secreted cytokines can activate B cells, cytotoxic T cells, macrophages, and other cells that participate in an immune response. Helper T cells or CD4⁺cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-γ, and TNF-β. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of particular value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response. An enhanced TH1 immune response may include one or more of an increase in CTLs, an increase in one or more of the cytokines associated with a TH1 immune response (such as IL-2, IFN-γ, and TNF-β), an increase in activated macrophages, an increase in NK activity, or an increase in the production of IgG2a. Preferably, the enhanced TH1 immune response will include an increase in IgG2a production. An enhanced TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune resonse will include an increase in IgG1 production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives hereof. Mineral containing compositions, such as aluminium salts are preferred TH2 adjuvants for use in the invention.

Preferably, the invention includes a composition comprising a combination of a TH1 adjuvant and a TH2 adjuvant. Preferably, such a composition elicits an enhanced TH1 and an enhanced TH2 response i.e. an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant (i.e. relative to immunization with a TH1 adjuvant alone or immunization with a TH2 adjuvant alone).

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

The use of an aluminium hydroxide or aluminium phosphate adjuvant is particularly preferred, and antigens are generally adsorbed to these salts.

The pH of compositions of the invention is preferably between 6 and 8, preferably about 7. Stable pH may be maintained by the use of a buffer. Where a composition comprises an aluminium hydroxide salt, it is preferred to use a histidine buffer [154]. The composition may be sterile and/or pyrogen-free. Compositions of the invention may be isotonic with respect to humans.

Compositions may be presented in vials, or they may be presented in ready-filled syringes. The syringes may be supplied with or without needles. A syringe will include a single dose of the composition, whereas a vial may include a single dose or multiple doses. Injectable compositions will usually be liquid solutions or suspensions. Alternatively, they may be presented in solid form (e.g. freeze-dried) for solution or suspension in liquid vehicles prior to injection.

Compositions of the invention may be packaged in unit dose form or in multiple dose form. For multiple dose forms, vials are preferred to pre-filled syringes. Effective dosage volumes can be routinely established, but a typical human dose of the composition for injection has a volume of 0.5 ml.

Where a composition of the invention is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Thus the invention provides for a kit comprising a first component and a second component, wherein: the first component comprises one or more polypeptide, antibody, vesicle and/or nucleic acid of the invention; and the second component comprises one or more of the following: instructions for administering a composition to a patient, a syringe or other delivery device, an adjuvant, and/or a pharmaceutically acceptable formulating solution.

The invention also provides a delivery device (e.g. a syringe) pre-filled with the immunogenic compositions of the invention.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials, and a typical quantity of each antigen per dose is between 0.1 μg and 1 mg per antigen.

### Nucleic Acid Immunisation

The immunogenic compositions described above include polypeptide antigens from UPEC. As an alternative to using proteins antigens in the immunogenic compositions of the invention, nucleic acid (preferably DNA e.g. in the form of a plasmid) encoding the antigen may be used, to give compositions, methods and uses based on nucleic acid immunisation. Nucleic acid immunisation is now a developed field (e.g. see references 155 to 162 etc.), and has been applied to many vaccines.

The nucleic acid encoding the immunogen is expressed in vivo after delivery to a patient and the expressed immunogen then stimulates the immune system. The active ingredient will typically take the form of a nucleic acid vector comprising: (i) a promoter; (ii) a sequence encoding the immunogen, operably linked to the promoter; and optionally (iii) a selectable marker. Preferred vectors may further comprise (iv) an origin of replication; and (v) a transcription terminator downstream of and operably linked to (ii). In general, (i) and (v) will be eukaryotic and (iii) and (iv) will be prokaryotic.

Preferred promoters are viral promoters e.g. from cytomegalovirus (CMV). The vector may also include transcriptional regulatory sequences (e.g. enhancers) in addition to the promoter and which interact functionally with the promoter. Preferred vectors include the immediate-early CMV enhancer/promoter, and more preferred vectors also include CMV intron A. The promoter is operably linked to a downstream sequence encoding an immunogen, such that expression of the immunogen-encoding sequence is under the promoter's control.

Where a marker is used, it preferably functions in a microbial host (e.g. in a prokaryote, in a bacteria, in a yeast). The marker is preferably a prokaryotic selectable marker (e.g. transcribed under the control of a prokaryotic promoter). For convenience, typical markers are antibiotic resistance genes.

The vector of the invention is preferably an autonomously replicating episomal or extrachromosomal vector, such as a plasmid.

The vector of the invention preferably comprises an origin of replication. It is preferred that the origin of replication is active in prokaryotes but not in eukaryotes.

Preferred vectors thus include a prokaryotic marker for selection of the vector, a prokaryotic origin of replication, but a eukaryotic promoter for driving transcription of the immunogen-encoding sequence. The vectors will therefore (a) be amplified and selected in prokaryotic hosts without polypeptide expression, but (b) be expressed in eukaryotic hosts without being amplified. This arrangement is ideal for nucleic acid immunization vectors.

The vector of the invention may comprise a eukaryotic transcriptional terminator sequence downstream of the coding sequence. This can enhance transcription levels. Where the coding sequence does not have its own, the vector of the invention preferably comprises a polyadenylation sequence. A preferred polyadenylation sequence is from bovine growth hormone.

The vector of the invention may comprise a multiple cloning site.

In addition to sequences encoding the immunogen and a marker, the vector may comprise a second eukaryotic coding sequence. The vector may also comprise an IRES upstream of said second sequence in order to permit translation of a second eukaryotic polypeptide from the same transcript as the immunogen. Alternatively, the immunogen-coding sequence may be downstream of an IRES.

The vector of the invention may comprise unmethylated CpG motifs e.g. unmethylated DNA sequences which have in common a cytosine preceding a guanosine, flanked by two 5′ purines and two 3′ pyrimidines. In their unmethylated form these DNA motifs have been demonstrated to be potent stimulators of several types of immune cell.

Vectors may be delivered in a targeted way. Receptor-mediated DNA therapy techniques are described in, for example, references 163 to 168. Therapeutic compositions containing a nucleic acid are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g. for enhancing or inhibiting levels of the encoded gene product) and efficacy of transformation and expression are considerations which will affect the dosage required for ultimate efficacy. Where greater expression is desired over a larger area of tissue, larger amounts of vector or the same amounts re-administered in a successive protocol of administrations, or several administrations to different adjacent or close tissue portions may be required to effect a positive therapeutic outcome. In all cases, routine experimentation in clinical trials will determine specific ranges for optimal therapeutic effect.

Vectors can be delivered using gene delivery vehicles. The gene delivery vehicle can be of viral or non-viral origin (see generally references 169 to 172).

Viral-based vectors for delivery of a desired nucleic acid and expression in a desired cell are well known in the art. Exemplary viral-based vehicles include, but are not limited to, recombinant retroviruses (e.g. references 173 to 183), alphavirus-based vectors (e.g. Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532); hybrids or chimeras of these viruses may also be used (e.g. U.S. Publication No. US20030148262; International Publication No. WO02/099035)), poxvirus vectors (e.g. vaccinia, fowlpox, canarypox, modified vaccinia Ankara, etc.), adenovirus vectors, and adeno-associated virus (AAV) vectors (e.g. see refs. 184 to 189). Administration of DNA linked to killed adenovirus [190] can also be employed.

Non-viral delivery vehicles and methods can also be employed, including, but not limited to, polycationic condensed DNA linked or unlinked to killed adenovirus alone [e.g. 190], ligand-linked DNA [191], eukaryotic cell delivery vehicles cells [e.g. refs. 192 to 196] and nucleic charge neutralization or fusion with cell membranes. Naked DNA can also be employed. Exemplary naked DNA introduction methods are described in refs. 197 and 198. Liposomes (e.g. immunoliposomes) that can act as gene delivery vehicles are described in refs. 199 to 203. Additional approaches are described in references 204 & 205.

Further non-viral delivery suitable for use includes mechanical delivery systems such as the approach described in ref. 205. Moreover, the coding sequence and the product of expression of such can be delivered through deposition of photopolymerized hydrogel materials or use of ionizing radiation [e.g. refs. 206 & 207]. Other conventional methods for gene delivery that can be used for delivery of the coding sequence include, for example, use of hand-held gene transfer particle gun or use of ionizing radiation for activating transferred genes [206 & 207].

Delivery DNA using PLG {poly(lactide-co-glycolide)} microparticles is a particularly preferred method e.g. by adsorption to the microparticles, which are optionally treated to have a negatively-charged surface (e.g. treated with SDS) or a positively-charged surface (e.g. treated with a cationic detergent, such as CTAB).

### Pharmaceutical Uses

The invention also provides a method of treating a patient, comprising administering to the patient a therapeutically effective amount of a composition of the invention. The patient may either be at risk from the disease themselves or may be a pregnant woman (‘maternal immunisation’ [209]).

The invention provides nucleic acid, polypeptide, vesicle or antibody of the invention for use as medicaments (e.g. as immunogenic compositions or as vaccines, or in a method of treating a patient) or as diagnostic reagents. It also provides the use of nucleic acid, polypeptide, vesicle or antibody of the invention in the manufacture of: (i) a medicament for treating or preventing disease and/or infection caused by an ExPEC bacterium; (ii) a diagnostic reagent for detecting the presence of or of antibodies raised against an ExPEC bacterium; and/or (iii) a reagent which can raise antibodies against an ExPEC bacterium. Said ExPEC bacterium can be of any serotype or strain. Preferably the ExPEC bacterium is a UPEC strain.

The invention is useful for the prevention and/or treatment of diseases such as bacteremia, meningitis, a urinary tract infection, pyelonephritis and/or cystitis. The invention is particularly useful for the treatment of urinary tract infections.

The patient is preferably a human. The human is preferably an adult (e.g. aged between 20 and 55). A vaccine intended for children or adolescents may also be administered to adults e.g. to assess safety, dosage, immunogenicity, etc. Female patients are a preferred subset, with sexually-active females aged 20-55 being a particularly preferred patient group. Another groups of patients is females aged 12-20, particularly for prophylactic use.

Other possible patient animals include dogs, which may be carriers of ExPEC [210,211].

One way of checking efficacy of therapeutic treatment involves monitoring infection after administration of the composition of the invention. One way of checking efficacy of prophylactic treatment involves monitoring immune responses against an administered polypeptide after administration. Immunogenicity of compositions of the invention can be determined by administering them to test subjects (e.g. children 12-16 months age, or animal models e.g. a mouse model) and then determining standard parameters including ELISA titres (GMT) of IgG. These immune responses will generally be determined around 4 weeks after administration of the composition, and compared to values determined before administration of the composition. Where more than one dose of the composition is administered, more than one post-administration determination may be made. Various mouse models of UTI are available [e.g. refs. 212 & 213-214].

Administration of polypeptide antigens is a preferred method of treatment for inducing immunity. Administration of antibodies of the invention is another preferred method of treatment. This method of passive immunisation is particularly useful for newborn children or for pregnant women. This method will typically use monoclonal antibodies, which will be humanised or fully human.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral (e.g. tablet, spray), vaginal, topical, transdermal, transcutaneous, intranasal, sublingual, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is 0.5 ml.

The invention may be used to elicit systemic and/or mucosal immunity. Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage treatment can be a single dose schedule or a multiple dose schedule. Multiple doses may be used in a primary immunisation schedule and/or in a booster immunisation schedule. A primary dose schedule may be followed by a booster dose schedule. In a multiple dose schedule the various doses may be given by the same or different routes e.g. a parenteral prime and mucosal boost, a mucosal prime and parenteral boost, etc. Suitable timing between priming doses (e.g. between 4-16 weeks), and between priming and boosting, can be routinely determined. For example, a primary course of vaccination may include 1-10 separate doses, followed by other doses given at subsequent time intervals required to maintain and/or reinforce an immune response, for example, at 1-4 months for a second dose, and if needed, a subsequent dose or doses after several months. A single dose schedule may comprise one administration or multiple administrations (collectively a single dose schedule). A multiple dose schedule comprises multiple doses, wherein each dose may comprise one administration or multiple administrations.

Bacterial infections affect various areas of the body and so compositions may be prepared in various forms. For example, the compositions may be prepared as injectables, either as liquid solutions or suspensions. Solid forms suitable for solution in, or suspension in, liquid vehicles prior to injection can also be prepared (e.g. a lyophilised or a spray-freeze dried composition). The composition may be prepared for topical administration e.g. as an ointment, cream or powder. The composition be prepared for oral administration e.g. as a tablet or capsule, as a spray or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as spray, drops, gel or powder [e.g. refs 215 & 216]. The composition may be in kit form, designed such that a combined composition is reconstituted just prior to administration to a patient. Such kits may comprise one or more antigens in liquid form and one or more lyophilised antigens.

Compositions of the invention may be administered to patients at substantially the same time as (e.g. during the same medical consultation or visit to a healthcare professional) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, a human papillomavirus vaccine, an inactivated poliovirus vaccine, a hepatitis B virus vaccine, a pneumococcal conjugate vaccine, a meningococcal conjugate vaccine, etc. Similarly, they may be administered to patients at substantially the same time as (e.g. during the same medical consultation or visit to a healthcare professional) an antibiotic, and in particular an antibiotic compound active against UPEC.

### Further Antigenic Components of Compositions of the Invention

The invention also provides a composition comprising a polypeptide or the invention and one or more of the following further antigens:
a saccharide antigen from N. meningitidis serogroup A, C, W135 and/or Y (preferably all four), such as the oligosaccharide disclosed in ref. 217 from serogroup C [see also ref. 218] or the oligosaccharides of ref. 219.
an antigen from N. meningitidis serogroup B such as those disclosed in refs. 220-228, etc.
a saccharide antigen from Streptococcus pneumoniae [e.g. 229, 230, 231].
an antigen from hepatitis A virus, such as inactivated virus [e.g. 232, 233].
an antigen from hepatitis B virus, such as the surface and/or core antigens [e.g. 233, 234].
an antigen from hepatitis C virus [e.g. 235].
an antigen from HIV [236]
a diphtheria antigen, such as a diphtheria toxoid [e.g. chapter 3 of ref. 237] e.g. the CRM₁₉₇ mutant [e.g. 238].
a tetanus antigen, such as a tetanus toxoid [e.g. chapter 4 of ref. 237].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 239 & 240].
a saccharide antigen from Haemophilus influenzae B [e.g. 218].
polio antigen(s) [e.g. 241, 242] such as IPV.
measles, mumps and/or rubella antigens [e.g. chapters 9, 10 & 11 of ref. 237].
varicella antigens.
influenza antigen(s) [e.g. chapter 19 of ref. 237], such as the haemagglutinin and/or neuraminidase surface proteins. Influenza antigens may be derived from interpandemic (annual) flu strains. Influenza antigens may be derived from strains with the potential to cause a pandemic outbreak (i.e., influenza strains with new haemagglutinin compared to the haemagglutinin in currently circulating strains, or influenza strains which are pathogenic in avian subjects and have the potential to be transmitted horizontally in the human population, or influenza strains which are pathogenic to humans). Influenza antigens may be derived from viruses grown in eggs or cell culture.
an antigen from Moraxella catarrhalis [e.g. 243].
a saccharide antigen from Streptococcus agalactiae (group B streptococcus).
an protein antigen from Streptococcus agalactiae (group B streptococcus) [e.g. 244-249]
an antigen from N. gonorrhoeae [e.g. 220-222 and 250].
an antigen from Chlamydia pneumoniae [e.g. refs. 251 to 257] or a combination of antigens from C. pneumoniae [e.g. 258].
an antigen from Chlamydia trachomatis, or a combination of antigens from C. trachomatis [e.g. 259].
an antigen from Porphyromonas gingivalis [e.g. 260].
rabies antigen(s) [e.g. 261] such as lyophilised inactivated virus [e.g. 262, RabAvert™]
antigen(s) from a paramyxovirus such as respiratory syncytial virus (RSV [263, 264]) and/or parainfluenza virus (PIV3 [265]).
an antigen from Bacillus anthracis [e.g. 266, 267, 268].
an antigen from Streptococcus pyogenes (group A streptococcus) [e.g. 245, 269, 270].
an antigen from Staphylococcus aureus [e.g. 271].
an antigen from a virus in the flaviviridae family (genus flavivirus), such as from yellow fever virus, Japanese encephalitis virus, four serotypes of Dengue viruses, tick-borne encephalitis virus, West Nile virus.
a pestivirus antigen, such as from classical porcine fever virus, bovine viral diarrhoea virus, and/or border disease virus.
a parvovirus antigen e.g. from parvovirus B 19.
a human papilloma virus (HPV) antigen [272]

The composition may comprise one or more of these further antigens.

In another embodiment, antigens of the invention are combined with one or more additional, non-E. coli antigens suitable for use in a vaccine designed to protect females against genitourinary and/or sexually transmitted diseases. For example, the antigens may be combined with an antigen derived from the group consisting of Streptococcus agalactiae, Chlamydia trachomatis, Neisseria gonorrhoeae, papillomavirus and herpes simplex virus. Where human papillomavirus antigens are used, they may be from one or more of the strains, HPV 16, HPV 18, HPV 6 and/or HPV 11.

Preferred gonococcal antigens include one or more of ngs13 (OmpA), OmpH, ngs576 (peptidyl-prolyl cis/trans isomerase (PPIase) protein), ngs41 and ngs117.

Preferred HPV antigens include one or more from the strains HPV 16, HPV 18, HPV 6 and HPV 11.

Preferred Chlamydia trachomatis antigens include one or more of: CT045, CT089, CT242, CT316, CT381. CT396, CT398, CT444, CT467, CT547, CT587, CT823, CT761 and specific combinations of these antigens as disclosed in reference273.

Preferred Chlamydia pneumoniae antigens include one or more of: CPn0324, Cpn0301, Cpn0482, Cpn0503, Cpn0525, Cpn0558, Cpn0584, Cpn0800, Cpn0979, Cpn0498, Cpn0300, Cpn0042, Cpn0013, Cpn450, Cpn0661, Cpn0557, Cpn0904, Clpn0795, Cpn0186 and Cpn0604 and specific combinations of these antigens as disclosed in reference274.

Preferred GBS antigens include one or more of GBS80, GBS104, GBS 59, GBS 67, GBS 322 and GBS 276.

In another embodiment, the antigen combinations of the invention are combined with one or more additional, non-ExPEC antigens suitable for use in a vaccine designed to protect elderly or immunocompromised individuals. For example, the antigen combinations may be combined with an antigen derived from the group consisting of Enterococcus faecalis, Staphylococcus aureus, Staphylococcus epidermis, Pseudomonas aeruginosa, Legionella pneumophila, Listeria monocytogenes, Neisseria meningitidies, influenza, and Parainfluenza virus (TIT).

Toxic protein antigens may be detoxified where necessary (e.g. detoxification of pertussis toxin by chemical and/or genetic means [240]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. Carrier proteins for the conjugates include bacterial toxins (such as diphtheria toxoid or tetanus toxoid), the N. meningitidis outer membrane protein [275], synthetic peptides [276,277], heat shock proteins [278,279], pertussis proteins [280,281], protein D from H. influenzae [282,283], cytokines [284], lymphokines [284], H. influenzae proteins, hormones [284], growth factors [284], toxin A or B from C. difficile [285], iron-uptake proteins [286], artificial proteins comprising multiple human CD4+T cell epitopes from various pathogen-derived antigens [287] such as the N19 protein [288], pneumococcal surface protein PspA [289], pneumolysin [290], etc. A preferred carrier protein is CRM197 protein [291].

Antigens in the composition will typically be present at a concentration of at least 1 μg/ml each. In general, the concentration of any given antigen will be sufficient to elicit an immune response against that antigen.

Antigens are preferably adsorbed to an aluminium salt.

### General

The term “comprising” encompasses “including” as well as “consisting” e.g. a composition “comprising” X may consist exclusively of X or may include something additional e.g. X+Y.

The term “about” in relation to a numerical value x means, for example, x+10%.

The word “substantially” does not exclude “completely” e.g. a composition which is “substantially free” from Y may be completely free from Y. Where necessary, the word “substantially” may be omitted from the definition of the invention.

The N-terminus residues in the amino acid sequences in the sequence listing are given as the amino acid encoded by the first codon in the corresponding nucleotide sequence. Where the first codon is not ATG, it will be understood that it will be translated as methionine when the codon is a start codon, but will be translated as the indicated non-Met amino acid when the sequence is at the C-terminus of a fusion partner. The invention specifically discloses and encompasses each of the amino acid sequences of the sequence listing having a N-terminus methionine residue (e.g. a formyl-methionine residue) in place of any indicated non-Met residue. It also specifically discloses and encompasses each of the amino acid sequences of the sequence listing starting at any internal methionine residues in the sequences.

The sequences disclosed herein were originally identified in the 536 strain. Genome sequences of several other strains of E. coli are available. Standard search and alignment techniques can be used to identify in any of these (or other) further genome sequences the homolog of any particular sequence of the present invention. Moreover, the sequences can be used to design primers for amplification of homologous sequences from other strains. Thus the invention is not limited to strain 536 sequences, but rather encompasses such variants and homologs from other strains of E. coli, particularly ExPEC and UPEC strains. In general, suitable variants of a particular SEQ ID NO include its allelic variants, its polymorphic forms, its homologs, its orthologs, its paralogs, its mutants, etc.

Thus, for instance, polypeptides used with the invention may, compared to the 536 sequence, include one or more (e.g. 1, 2, 3, 4, 5, 6, 7, 8, 9, etc.) amino acid substitutions, insertions, deletions, etc. as disclosed above.

As indicated in the above text, nucleic acids and polypeptides of the invention may include sequences that:
(a) are identical (i.e. 100% identical) to the sequences disclosed in the sequence listing;
(b) share sequence identity with the sequences disclosed in the sequence listing;
(c) have 1, 2, 3, 4, 5, 6, 7, 8, 9 or 10 single nucleotide or amino acid alterations (deletions, insertions, substitutions), which may be at separate locations or may be contiguous, as compared to the sequences of (a) or (b); and
(d) when aligned with a particular sequence from the sequence listing using a pairwise alignment algorithm, a moving window of x monomers (amino acids or nucleotides) moving from start (N-terminus or 5′) to end (C-terminus of 3′), such that for an alignment that extends to p monomers (where p>x) there are p−x+1 such windows, each window has at least xy identical aligned monomers, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if xy is not an integer then it is rounded up to the nearest integer. The preferred pairwise alignment algorithm is the Needleman-Wunsch global alignment algorithm [292], using default parameters (e.g. with Gap opening penalty=10.0, and with Gap extension penalty=0.5, using the EBLOSUM62 scoring matrix). This algorithm is conveniently implemented in the needle tool in the EMBOSS package [293].

The nucleic acids and polypeptides of the invention may additionally have further sequences to the N-terminus/5′ and/or C-terminus/3′ of these sequences (a) to (d).

The practice of the present invention will employ, unless otherwise indicated, conventional methods of chemistry, biochemistry, molecular biology, immunology and pharmacology, within the skill of the art. Such techniques are explained fully in the literature. See, e.g., references 294-301, etc.

In some embodiments, the invention does not encompass the polypeptides disclosed in reference 27, reference 28, U.S. Provisional Application No. 60/654,632 (filed Feb. 18, 2005; priority application for refs 27 & 28) or U.S. Provisional Application No. 60/712,720 (filed Aug. 29, 2005; priority application for ref. 28).

### MODES FOR CARRYING OUT THE INVENTION

Below are examples of specific embodiments or modes for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

Computer-based comparative and predictive tools were used to identify 93 polypeptides from the UPEC 536 strain, using criteria such as (a) less than 90% sequence identity with two commensal strains (mg1655, DH10B), (b) have a length greater than 100 amino acids, (c) have a non-cytoplasmic cellular localisation, and (d) are common to another UPEC strain (CFT073) but are not found in an MNEC strain (1HE3034). These sequences are listed in the sequence listing. Their amino acid sequences are SEQ ID NOS: 8 to 100.

In parallel studies, assays were carried out (see below) in order to select surface-exposed proteins, which are specific for UPEC strains and absent in non-pathogenic strains (commensal and laboratory strains). 76 polypeptides were identified, 11 of which had less than 80% homology with commensal strains (mg1655). The sequences of these 11 polypeptides are listed in the sequence listing and their amino acid sequences are SEQ ID NOS 1 to 10 and SEQ ID NO 85. The sequences of 7 of these polypeptides were identified by this method but not by the computer-based method. These sequences are listed in the sequence listing and their amino acid sequences are SEQ ID NOS 1 to 7. Table 1 shows the 76 polypeptides that were identified in this way (SEQ ID NOs 1 to 10, 85 and 101-165).

In further work, SEQ ID NOs 166 and 167 were identified.

SEQ ID NOs 1-167 are listed in the sequence listing. They are also referred to by ‘RECP’ nomenclature, as shown in Table 4.

The invention provides a credible utility for these polypeptides, namely in the provision of immunogenic compositions as described herein.

These polypeptides may be cloned, expressed and purified. The purified antigens may then be used to immunise mice, whose sera can be analysed by Western blot, ELISA and FACS, and further tested in both in vitro and in vivo experiments. Suitable in vitro experiments include testing the ability of antibodies to induce complement-mediated bacterial killing and/or opsonophagocytosis activity, to block binding of ExPEC strains (or the purified antigen) to human epithelial cells (e.g. in bladder cells) or other cell lines, and/or to inhibit adhesion/invasion of E. coli bacteria (e.g. K1 strain) to brain microvascular endothelial cells (BMEC). Suitable in vivo experiments include active and/or passive systemic immunisations and challenge in mouse models of UTI (adult mice), protection by active or passive immunisations against bacteremia and meningitis in 5-day-old rats challenged with E. coli K1 strain, and immunisation and intraperitoneal infection of adult mice with an ExPEC strain.

The importance of the proteins to the bacterial life-cycle may be tested by creating isogenic knockout mutants. The mutants can also be used to ensure that sera raised by an antigen are specific for that antigen. Microarrays may be used to study expression patterns. Conservation and/or variability is assessed by sequencing the genes from multiple different ExPEC strains. The invention also provides an E. coli in which one or more of the polypeptides of the invention has/have been knocked out. A knockout mutation may be situated in the coding region of the gene or may lie within its transcriptional control regions (e.g. within its promoter). A knockout mutation will reduce the level of mRNA encoding the antigen to <1% of that produced by the wild-type bacterium, preferably <0.5%, more preferably <0.1%, and most preferably to 0%.

Proteomic assays were carried out in order to select predicted surface-exposed proteins, which are specific for UPEC strains and absent in non-pathogenic strains (commensal and laboratory strains). Once selected these proteins may be expressed and purified and used to immunize mice.

It is known from reference 43 that a mutation in any of the tol-pal genes of E. coli results in the formation of vesicles containing native outer membrane proteins. Proteins present in vesicles of UPEC strains and which had low homology to commensal strains such as mg1655 were selected as a small potential group of proteins that could be used as potential antigens.

### Lambda Red-Mediated Gene Manipulation in Commensal and Pathogenic E. Coli

This method is a rapid PCR-based method used to inactivate the to 1R gene from the wild-type E. coli strains [302]. Briefly, the first step consists in amplifying independently the upstream and downstream regions of the target gene (toIR) and the resistance marker cassette. The two PCR products obtained in step 1 are mixed with the amplification producer of the AB cassette at equimolar concentrations and submitted to a second round of PCR (a three way PCR) to generate a resistance marker cassette flanked by upstream and downstream 500 bp (or more) regions homologous to the target gene. In the third step, large amounts (1 μg) of the desired linear DNA are electroporated into lamda-red competent cells.

### Vesicle Preparation

1. Vesicle Preparation by Precipitation with TCA

LB media was inoculated with bacteria grown on plates and incubated overnight at 37° C. under gentle shaking. The culture was used to inoculate 200 ml of LB at OD600 0.1. Bacteria were grown to OD600 0.4 (or as specified). Culture was centrifuged for 10 minutes at 4000×g and the supernatant was filtered through a 0.22 mm filter to remove residual bacteria.

The same experiments were also performed under iron limiting conditions by adding Dipyridyl (0.25 mM) to the LB media.

Precipitation was performed by adding to the culture supernatant 10% final of a solution at 100% (w/v) TCA, 0.4% (w/v) deoxycholate. The precipitation was allowed to proceed for 30 minutes at 4° C. Precipitate was recovered by 10 minutes centrifugation at 20000×g at 4° C. The pellet was washed once with 10% TCA (w/v) and twice with absolute ethanol. The pellet was dried with speed vac, and stored at −20° C.

The wild type and mutated strains were subjected to SDS polyacrylamide gel electrophoresis from which it could be observed that there were many more bands in the supernatant of the mutated strains than the wildtype strains. Randomly picked bands demonstrated that most of the proteins in the supernatant were membrane proteins, indicating enrichment in membrane content.

### 2. Vesicle Preparation by Ultracentrifugation

Culture supernatant was ultracentrifuged at 200000×g for 2 hours at 4° C. The pellet was washed with PBS, resuspended in PBS, and stored at −20° C.

### 3. Guanidinium Denaturation of the Vesicles

Prior to the guanidinium denaturation, Vesicles were precipitated with ethanol. 10 μg of OMV in PBS were precipitate by adding cold absolute ethanol to 90% final. Precipitation was allowed to proceed for 20 minutes at −20° C. Precipitate was recovered by 10 minutes centrifugation at 13000×g. Pellet was resuspended with 50 ml, 6M guanidinium, 15 mM DTT, 200 mM Tris-HCl, pH 8.0. Denaturation was allowed to proceed for 60 minutes at 60° C. Prior to digestion, solution was diluted 1/8 with a solution of 1.5M Tris pH 8.0 and 5 mg of trypsin were added to the diluted solution. Digestion was allowed to proceed overnight at 37° C. Reaction was stopped by adding 0.1% final of formic acid. Peptides were extracted using Oasis extraction cartridges. Peptides were analyzed by LC coupled MS-MS.

### 4. Sun Face Digestion

5 mg of trypsin were added to 10 mg of vesicles in PBS and incubated at 37° C. for 3 hours. Reaction was stopped by adding 0.1% final of formic acid. Peptides were recovered and desalted with Oasis extraction cartridge. Peptides were analyzed with LC coupled MSMS.

### Vesicle Analysis

### Protein Quantification

Proteins were quantified with the Bradford method, using the BSA as standard.

### SDS-Page

Samples were analyzed with a sodium dodecyl sulfate (SDS) 4-12% polyacrylamide gel, using a Mini-Protean II electrophoresis apparatus. Samples were suspended in SDS sample buffer (0.06 M Tris-HCl pH 6.8, 10% (v/v) glycerol, 2% (w/v) SDS, 5% (v/v) 2-mercaptoethanol, 10 mg/ml bromophenol blue) and heated to 100° C. for 5 min before SDS-polyacrylamide gel electrophoreis. After the run, gels were stained with Coomassie Blue

### MALDI-TOF Mass Spectrometry.

Protein bands or spots were excised from gels, washed with 50 mM ammonium bicarbonate/acetonitrile (50/50, v/v) twice, washed once with pure acetonitrile and air-dried. The dried spots were digested at 37° C. for 2 h by adding 7 to 10 ml of a solution containing 5 mM ammonium bicarbonate, 0.012 mg of sequencing-grade trypsin. After digestion 0.6 ml were loaded on a matrix pre-spotted target and air-dried. Spots were washed with 0.6 ml of a solution of 70% ethanol, 0.1% trifluoracetic acid. Mass spectra were acquired on an ultraflex MALDI TOF mass spectrometer. Spectra were externally calibrated by using a combination of standards pre-spotted on the target. Protein identification was carried out by both automatic and manual comparisons of experimentally generated monoisotopic peaks of peptides in the mass range of 700 to 3,000 Da with computer-generated fingerprints, using the Mascot program.

### Bi-Dimensional Electrophoresis

200 mg of vesicles were resuspended in an Immobiline re-swelling solution (7M urea, 2M thiourea, 2% (w/v) CHAPS (2% w/v) ASB14, 2% (v/v) IPG buffer pH 3-10 NL, 2 mM TBP, 65 mM DTT), and adsorbed overnight on 7 cm Immobiline DryStrips (pH 3-10 NL). Proteins were then separated by 2D electrophoresis. The first dimension was run using a IPGphor Isoelectric Focusing Unit, applying sequentially 150 V for 35 minutes, 500 V for 35 minutes, 1,000 V for 30 minutes, 2,600 V for 10 minutes, 3,500 V for 15 minutes, 4,200 V for 15 minutes, and finally 5,000 V to reach 10 kVh. For the second dimension, the strips were equilibrated by two 10 minute -incubations in 4 M urea, 2 M thiourea, 30% glycerol, 2% SDS, 5 mM TBP, 50Mm Tris HCl pH 8.8, 2.5% acrylamide, Bromo phenol Blue 0.2%: Proteins were then separated on linear 4-12% precasted polyacrylamide gels.

Gels were stained with colloidal Coomassie Blue and scanned with a Personal Densitometer SI. Images were analyzed with Image Master 2D Elite software.

### Nano-LC/MS/MS

Peptides were separated by nano-LC on a CapLC HPLC system connected to a Q-ToF Micro ESI mass spectrometer equipped with a nanospray source. Samples were loaded onto an Atlantis C18 NanoEase column (100 μm i.d.×100 mm), through a C18 trap column (300 μm i.d.×5 mm). Peptides were eluted with a 50-min gradient from 2% to 60% of 95% ACN, in a solution of 0.1% formic acid at a flow rate of 400 nl/minute. The eluted peptides were subjected to an automated data-dependent acquisition program, using the MassLynx software, version 4.0, where a MS survey scan was used to automatically select multi-charged peptides over the m/z range of 400-2,000 for further MS/MS fragmentation. Up to three different components where subjected to MS/MS fragmentation at the same time. After data acquisition, the individual MS/MS spectra were combined, smoothed and centroided by MassLynx. Search and identification of peptides were performed in batch mode with a licensed version of MASCOT. The MASCOT search parameters were: (1) species: ExPEC (2) allowed number of missed cleavages (only for trypsin digestion): 6; (3) variable post-translational modifications: methionine oxidation; (4) peptide tolerance: ±500 ppm; (5) MS/MS tolerance: ±0.3 Da and (6): peptide charge: from +1 to +4. As for the previous platform, only significant hits as defined by MASCOT probability analysis were considered. The score thresholds for acceptance of protein identifications from at least one peptide were set by MASCOT as 18 for trypsin digestion and 36 for proteinase K digestion.

### Results

As a result of the above analyses, 76 antigens were identified from the 536 strain as shown in Table 1.11 of these sequences had a very low homology with commensal strains (mg1655) (SEQ ID NOS 1-10 and SEQ ID NO 85). The sequences of 7 of these polypeptides were identified by this method but not by the computer-based method. These sequences are listed in the sequence listing and their amino acid sequences are SEQ ID NOS 1 to 7. Table 1 shows the 76 polypeptides that were identified in this way.

### Antigen Analysis

### Mouse Model of Systemic Infection

To screen a large number of antigens selected by comparative genome analysis between pathogenic and non pathogenic E. coli strains, a protection model based on a classical virulence assay has been established. Alternative experimental models that may also be used include those outlined in references 212-214.

The experimental model (immunization and infection) uses 5 week old—CD 1 outbreed mice which are challenged with intravenous inoculation of virulent UPEC 536 E. coli strain. The challenge dose has been experimentally determined as the amount of bacteria able to kill 80% of adult mice within 4 days and corresponds to 4×10⁷ cfu/mouse for the 536 strain.

### Immunization Protocol

A large number of antigens from UPEC 536 strain were cloned, expressed and purified. In addition SEQ ID NO 168, which corresponds to the N-terminal region (amino acids 21-470) of SEQ ID NO 56, was cloned, expressed and purified. The purified antigens were used to immunize mice in the experimental mouse model in the following way. Mice are immunized three times by subcutaneous injection of 150 μl of protein solution using freund's adjuvants as shown in the table below:

| | Control mice: | Immunized mice: |
|---|---|---|
| Day 0 | 75 μl of saline solution | 75 μl of protein solution (20 μg) |
| | 75 μl of complete | 75 μl of complete freund's adjuvant |
| | freund's adjuvant | |
| Day 21 | 75 μl of saline solution | 75 μl of protein solution (20 μg) |
| | 75 μl of incomplete | 75 μl of incomplete freund's adjuvant |
| | freund's adjuvant | |
| Day 35 | 75 μl of saline solution | 75 μl of protein solution (20 μg) |
| | 75 μl of incomplete | 75 μl of incomplete freund's adjuvant |
| | freund's adjuvant | |

Blood samples are colleccted the day before the first immunization (preimmune serum), at day 34 and 48 (day before challenge). Sera from immunized animals are tested by western blot and ELISA to determine the antibodies titer.

### Challenge

At day 48 E. coli UPEC 536 strain is streaked on LB agar plate from frozen stock and incubated overnight (ON) at 37° C. in incubator. At Day 49 the ON plate-culture is used to inoculate 50 ml of LB medium to have an O.D.₆₀₀=0.1, and grown for 1.5 hours at 37° C. under agitation until the bacterial culture reaches an O.D.₆₀₀=1.3 which corresponds to 4×10⁸ cfu/ml for the UPEC 536 strain. The culture is centrifuged and the pellet resuspended in the same volume with physiological solution and used for challenge undiluted. The culture is plated using a standard plate count method to verify the inoculum. 100 μl of the cell suspension containing 4×10⁷UPEC 536 bacteria is injected intravenously, using a 1 ml syringe, to control and immunized mice. The number of deaths in each animal group at 24, 48, 72 and 96 hours after infection are recorded.

The protection due to vaccination is evaluated by comparison of the survival in the vaccinated group and the survival in control group of mice at 96 hours from the challenge. Percentage of survival relative to controls is calculated using the formula:

$$\frac{\begin{matrix}rate of deaths in vaccine group - \\ rate of deaths in control group\end{matrix}}{rate of deaths in control group}$$

### Results

The results can be seen in Table 3 which demonstrates that the % survival of the mice after challenge with UPEC 536 is increased following immunization with antigens of the invention.

It will be understood that the invention has been described by way of example only and modifications may be made whilst remaining within the scope and spirit of the invention.

TABLE 1

| Polypeptide from | | | |
|---|---|---|---|
| strain 536 | Length | Annotation | Psort |
| recp00182 | 810 | Hypothetical Protein | outer membrane |
| recp00955 | 346 | Outer membrane protein A precursor | periplasmic space |
| recp03768 | 1024 | Hemolysin A | inner membrane |
| recp03614 | 219 | Outer membrane protein | outer membrane |
| recp04758 | 394 | Elongation factor Tu-A | cytoplasm |
| recp00156 | 747 | Ferrichrome-iron receptor | outer membrane |
| recp00821 | 171 | Outer membrane protein X precursor | outer membrane |
| recp01147 | 360 | Outer membrane porin protein LC | outer membrane |
| | | precursor | |
| recp02564 | 245 | LIPOPROTEIN, COML FAMILY | outer membrane |
| recp03085 | 493 | Outer membrane protein tolC | outer membrane |
| recp00198 | 236 | COPPER HOMEOSTASIS PROTEIN | outer membrane |
| | | CUTF | |
| recp00751 | 173 | Peptidoglycan-associated lipoprotein | outer membrane |
| recp02152 | 332 | D-galactose-binding protein | inner membrane |
| recp02221 | 375 | Outer membrane protein C precursor | outer membrane |
| recp03591 | 1157 | Cellulose synthase operon C protein | periplasmic space |
| Recp02237 | 562 | Hypothetical Protein | periplasmic space |
| Recp00167 | 474 | PROTEASE DO (EC 3.4.21.—) | outer membrane |
| Recp02453 | 344 | Lipoprotein-34 precursor | outer membrane |
| Recp00092 | 420 | Cell division protein ftsA | inner membrane |
| Recp00183 | 161 | Histone-like protein HLP-1 precursor | periplasmic space |
| Recp01092 | 213 | putative lipoprotein | outer membrane |
| Recp02481 | 392 | Hypothetical Protein | outer membrane |
| Recp03191 | 678 | GS60 ANTIGEN | outer membrane |
| Recp03194 | 191 | Hypothetical Protein | outer membrane |
| Recp03399 | 270 | Peptidyl-prolyl cis-trans isomerase (EC | periplasmic space |
| | | 5.2.1.8) | |
| Recp04801 | 71 | Hypothetical Protein | periplasmic space |
| Recp00778 | 427 | Putative lipoprotein ybHC precursor | outer membrane |
| Recp01607 | 700 | Acetyl-CoA:acetoacetyl-CoA | cytoplasm |
| | | transferase alpha subunit (EC 2.8.3.—) | |
| Recp01205 | 270 | Septum site-determining protein minD | cytoplasm |
| Recp01576 | 155 | Outer membrane lipoprotein slyB | outer membrane |
| | | precursor | |
| Recp02965 | 948 | Antigen 43 precursor | outer membrane |
| Recp00323 | 1042 | Antigen 43 precursor | inner membrane |
| recp00299 | 725 | Hypothetical Protein | outer membrane |
| recp00752 | 263 | Hypothetical Protein | periplasmic space |
| recp00933 | 362 | Outer membrane protein F precursor | outer membrane |
| recp01276 | 891 | Aldehyde-alcohol dehydrogenase | inner membrane |
| recp01416 | 539 | Protein ydcG precursor | inner membrane |
| recp01834 | 167 | Ferritin-like protein 2 | cytoplasm |
| recp01846 | 555 | Flagellin | cytoplasm |
| recp02458 | 487 | Hypothetical Protein | inner membrane |
| recp02979 | 558 | Polysialic acid transport protein kpsD | outer membrane |
| recp03533 | 524 | Dipeptide-binding protein | periplasmic space |
| recp03742 | 409 | Hypothetical Protein | inner membrane |
| recp03841 | 471 | Tryptophanase (EC 4.1.99.1) | cytoplasm |
| recp04121 | 628 | Vitamin B12 receptor precursor | inner membrane |
| recp04474 | 1024 | Hemolysin, chromosomal | inner membrane |
| recp04553 | 878 | Outer membrane usher protein fimD | inner membrane |
| recp04625 | 645 | Soluble lytic murein transglycosylase | periplasmic space |
| | | (EC 3.2.1.—) | |
| recp04667 | 274 | Putative ATP binding protein SugR | cytoplasm |
| recp01536 | 314 | Protein ydgH precursor | periplasmic space |
| recp00086 | 438 | UDP-N-acetylmuramoylalanine--D- | inner membrane |
| | | glutamate ligase (EC 6.3.2.9) | |
| Recp00769 | 229 | Molybdenum transport system | inner membrane |
| | | permease protein modB | |
| Recp01006 | 363 | Putative monooxygenase ycdM | inner membrane |
| Recp01611 | 78 | Major outer membrane lipoprotein | outer membrane |
| | | precursor | |
| Recp01750 | 518 | Hypothetical Protein | inner membrane |
| Recp02349 | 446 | Long-chain fatty acid transport protein | outer membrane |
| | | precursor | |
| Recp04188 | 446 | Maltoporin precursor | outer membrane |
| Recp00053 | 237 | Hypothetical Protein | cytoplasm |
| Recp00054 | 191 | Peptidyl-prolyl cis-trans isomerase (EC | periplasmic space |
| | | 5.2.1.8) | |
| Recp00118 | 630 | Dihydrolipoamide acetyltransferase | inner membrane |
| | | component of pyruvate dehydrogenase | |
| Recp00535 | 550 | Protein ushA precursor | periplasmic space |
| Recp00750 | 430 | TolB protein | outer membrane |
| Recp01673 | 112 | Osmotically inducible lipoprotein E | outer membrane |
| | | precursor | |
| Recp02416 | 191 | putative lipoprotein | outer membrane |
| Recp02876 | 230 | PERIPLASMIC IMMUNOGENIC | cytoplasm |
| | | PROTEIN | |
| Recp03068 | 130 | Protein ygiW precursor | periplasmic space |
| recp03340 | 127 | LSU ribosomal protein L17P | cytoplasm |
| recp03342 | 206 | SSU ribosomal protein S4P | cytoplasm |
| recp03556 | 188 | Outer membrane protein slp precursor | outer membrane |
| recp04322 | 548 | 60 kDa chaperonin | cytoplasm |
| recp04464 | 205 | PapA protein | outer membrane |
| recp04606 | 201 | Osmotically inducible protein Y | periplasmic space |
| | | precursor | |
| recp00609 | 749 | Ferrienterobactin receptor precursor | outer membrane |
| recp02100 | 350 | Fructose-bisphosphate aldolase class I | cytoplasm |
| recp02302 | 714 | Phosphate acetyltransferase | inner membrane |
| recp03559 | 660 | Hemin receptor | outer membrane |

TABLE 2
7 preferred pathogenic E.coli 536 strain sequences

SEQ ID NO: 1
(RECP04801)
MTMKLIKTLVAVSALSMMSFGVFAQSVSATASTLDRAEAKIAAQAAEQGASYKITSARVENRVYMTAELLK
SEQ ID NO: 2
(RECP04667)
VVNGHTDVIGSTSIRHILAVRQSTLLQIDTLIRQLAEISAMTVSIGGKTALDWAMKQDFRCGFWLMEKPEIAMKAIT
RNLDRELWRDLMQRSGMLSLMDAQARETWYRSLEYDNVPEISEANILNTFKQLHQNKDEVFERGVINVFRGLNWNYK
TNLPCKFGSKIIVNNLVRWDRWGFHIVTGQQADRVADLERMLHLFSGWPIPDNRENIIIRLDDHIQSAQGQECYEYE
DEMFSIRYFKKGSAHITFRKPELIDRLNDITAKYYPEIIPYNI
SEQ ID NO: 3
(RECP03768)
MPTITTAQIKSTLQSAKQSAANKLHSAGQSTKDALKKAAEQTRNAGNRLILLIPKDYKGQGSSLNDLVRTADELGIE
VQYDEKNGTAITKQVFGTAEKLIGLTERGVTIFAPQLDKLLQKYQKAGNKLGGSAENIGDNLGKAGSVLSTFQNFLG
TALSSMKIDELIKKQKSGSNVSSSELAKASIELINQLVDTAASINNNVNSFSQQLNKLGSVLSNTKHLNGVGNKLQN
LPNLDNIGAGLDTVSGILSVISASFILSNADADTGTKAAAGVELTTKVLGNVGKGISQYIIAQRAAQGLSTSAAAAG
LIASAVTLAISPLSFLSIADKFKRANKIEEYSQRFKKLGYDGDSLLAAFHKETGAIDASLTTISTVLASVSSGISAA
ATTSLVGAPVSALVGAVTGIISGILEASKQAMFEHVASKMADVIAEWEKKHGKNYFENGYDARHAAFLEDNFKILSQ
YNKEYSVERSVLITQQHWDTLIGELAGVTRNGDKTLSGKSYIDYYEEGKRLEKKPDEFQKQVFDPLKGNIDLSDSKS
STLLKFVTPLLTPGEEIRERRQSGKYEYITELLVKGVDKWTVKGVQDKGSVYDYSNLIQHASVGNNQYREIRIESHL
GDGDDKVFLAAGSANIYAGKGHDVVYYDKTDTGYLTIDGTKATEAGNYTVTRVLGGDVKVLQEVVKEQEVSVGKRTE
KTQYRSYEFTHINGTDLTETDNLYSVEELIGTNRADKFFGSKFTDIFHGADGDDHIEGNDGNDRLYGDKGNDTLRGG
NGDDQLYGGDGNDKLTGGVGNNYLNGGDGDDELQVQGNSLAKNVLSGGKGNDKLYGSEGADLLDGGEGNDLLKGGYG
NDIYRYLSGYGHHIIDDDGGKDDKLSLADIDFRDVAFKREGNDLIMYKAEGNVLSIGHKNGITFRNWFEKESGDISN
HQIEQIFDKDGRVITPDSLKKAFEYQQSNNQANYVYGEYASTYADLDNLNPLINEISKIISAAGNFDVKEERSAASL
LQLSGNASDFSYGRNSITLTASA
SEQ ID NO: 4
(RECP03742)
MELIMPLSRRNFIQNAVLGISAAGLSAAPALAKNISSSTAHIISKTSGHADTSTSKSLHIISLDRLEASAKDVMTEA
AYAYIARGAGDEWTYHENRRAFSDYPLLPHRLSGVAAHSIDIRTDLLGHHLEHPLLIAPMGAHMFVHPEGEVIAAAG
AEKAGALYESSGASNRSLEDIAKASKGPKWFQLYFNADAGVTRSLLERAKAAGYSAIIITADALGPGTSDAFLSMSS
PFPAGATFGNHDPRYGGKGDFFNQKVELTPADIEFVKKITGLPVIVKGILRGEDAVVAIDAGADAIQVSNHGGRQID
GVPSAISQLQEVAARVGHKVPVIFDSGIRRGIDVVRAISLGATAVAVGRPVLYGIAAGGVGGVASVIEHLKTELRTA
MLLSGARTLKDLSQGFIRNKETEH
SEQ ID NO: 5
(RECP01846)
MAQVINTNSLSLITQNNINKNQSALSSSIERLSSGLRINSAKDDAAGQAIANRFTSNIKGLTQAARNANDGISVAQT
TEGALSEINNNLQRIRELTVQASTGTNSDSDLDSIQDEIKSRLDEIDRVSGQTQFNGVNVLAKDGSMKIQVGANDGQ
TITIDLKKIDSDTLGLSGFNVNGKGAVANTAATKDDLVAASVSAAVGNEYTVSAGLSKSTAADVIASLTDGATVTAA
GVSNGFAAGATGNAYKFNQANNTFTYNTTSTAAELQSYLTPKAGDTATFSVEIGSTKQDVVLASDGKITAKDGSKLY
IDTTGNLTQNGGGTLEEATLNGLAFNHSGPAAAVQSTITTADGTSIVLAGSGDEGTTKTAGAINVTGAVISADALLS
ASKATGFTSGAYTVGTDGVVKSGGNDVYNKADGTGLTTDNTTKYYLQDDGSVTNGSGKAVYVDATGKLTTDAETKAA
TTADPLKALDEAISSIDKERSSLGAVQNRLDSAVTNLNNTTTNLSEAQSRIQDADYATEVSNMSKAQIIQQAGNSVL
AKANQVPQQVLSLLQG
SEQ ID NO: 6
(RECP03559)
MSRPQFTSLRLSLLALAVSATLPTFAFATETMTVTATGNARSSFEAPMMVSVIDTSAPENQTATSATDLLRYVPGIT
LDGTGRTNGQDVNMRGYDHRGVLVLVDGVRQGTDTGHLNGTFLDPALIKRVEIVRGPSALLYGSGALGGVISIDTVD
AKDLLQEGQSSGFRVFGTGGTGDHSLGLGASAFGRTENLDGIVAWSSRDRGDLRQSNGETAPNDESINNMLAKGTWQ
IDSAQSLSGLVRYYNNDAREPKNPQTVEASDSSNPMVDRSTIQRDAQLSYKLAPQGNDWLNADAKIYWSEVRINAQN
TGSSGEYREQITKGARLENRSTLFADSFASHLLTYGGEYYRQEQHPGGATTGFPQAKIDFSSGWLQDEITLRDLPIT
LLGGTRYDSYRGSSDGYKDVDADKWSSRAGMTINPTNWLMLFGSYAQAFRAPTMGEMYNDSKHFSIGREYTNYWVPN
PNLRPETNETQEYGFGLRFDDLMLSNDALEFKASYFDTKAKDYISTTVDFAAATTMSYNVPNAKIWGWDVMTKYTTD
LFSLDVAYNRTRGKDTDTGEYISSINPDTVTSTLNIPIAHSGFSVGWVGTFADRSTHISSSYSKQPGYGVNDFYVSY
QGQQALKGMTTTLVLGNAFDKEYWSPQGIPQDGRNGKIFVSYQW
SEQ ID NO: 7
(RECP04474)
MPTITTAQIKSTLQSAKQSAANKLHSAGQSTKDALKKAAEQTRNAGNRLILLIPKDYKGQGSSLNDLVRTADELGIE
VQYDEKNGTAITKQVFGTAEKLIGLTERGVTIFAPQLDKLLQKYQKAGNKLGGSAENIGDNLGKAGSVLSTFQNFLG
TALSSMKIDELIKKQKSGGNVSSSELAKASIELINQLVDTAASLNNNVNSFSQQLNKLGSVLSNTKHLNGVGNKLQN
LPNLDNIGAGLDTVSGILSAISASFILSNADADTGTKAAAGVELTTKVLGNVGKGISQYIIAQRAAQGLSTSAAAAG
LIASVVTLAISPLSFLSIADKFKRANKIEEYSQRFKKLGYDGDSLLAAFHKETGAIDASLTTISTVLASVSSGISAA
ATTSLVGAPVSALVGAVTGIISGILEASKQAMFEHVASKMADVIAEWEKKHGKNYFENGYDARHAAFLEDNFEILSQ
YNKEYSVERSVLITQQHWDTLIGELAGVTRNGDKTLSGKSYIDYYEEGKRLEKEPDEFQKQVFDPLKGNIDLSVIKS
STLLKFITPLLTPGKEIRERRQSGKYEYITELLVKGVDKWTVKGVQDKGSVYDYSNLIQHASVGNNQYREIRIESHL
GDGDDKVFLSAGSANIYAGKGHDVVYYDKTDTGYLTIDGTKATEAGNYTVTRVLGGDVKVLQEVVKEQEVSVGKRTE
KTQYRSYEFTHINGTDLTETDNLYSVEELIGTNRADKFFGSKFTDIFHGADGDDHIEGNDGNDRLYGDKGNDTLRGG
NGDDQLYGGDGNDKLTGGVGNNYLNGGDGDDELQVQGNSLAKNVLSGGKGNDKLYGSEGADLLDGGEGNDLLKGGYG
NDIYRYLSGYGHHIIDDDGGKDDKLSLADIDFRDVAFKREGNDLIMYKAEGNVLSIGHKNGITFRNWFEKESGDISN
HQIEQIFDKDGRVITPDSLKKAFEYQQSNNQANYVYGEYASTYADLDNLNPLINEISKIISAAGNFDVKEERSAASL
LQLSGNASDFSYGRNSITLTASA

TABLE 3

| | | | Animal model | | |
|---|---|---|---|---|---|
| | | SEQ | survival | Survival | |
| | | ID | immun. | ctrl. | Protection |
| Antigens | Annotation | NO: | (%) | (%) | % |
| Recp02040 | Lipopolysaccharide | 95 | 6/10 (60) | 3/10 (30) | 43 |
| | N-acetylglucosaminyltransferase | | | | |
| Recp03760 | Hypothetical Protein | 80 | 4/10 (40) | 1/10 (10) | 33 |
| Recp03740 | F17 fimbrial protein precursor | 73 | 5/10 (50) | 3/10 (30) | 28.5 |
| Recp03761 | Hypothetical Protein | 81 | 5/10 (50) | 3/10 (30) | 28.5 |
| Recp02934 | PapH protein | 65 | 5/10 (50) | 3/10 (30) | 28.5 |
| Recp01398A | Putative surface- | 166 | 3/8 (37.5) | 1/10 (10) | 30 |
| | exposed virulence protein bigA | | | | |
| Recp04460 | PapJ protein | 98 | 4/8 (50) | 1/10 (10) | 44 |
| Recp04462 | PapC protein | 15 | 6/8 (75) | 1/10 (10) | 72 |
| Recp04463 | PapH protein | 167 | 3/8 (37.5) | 1/10 (10) | 30 |
| Recp04475 | Hemolysin C | 168 | 4/8 (50) | 1/10 (10) | 44 |

TABLE 4

| SEQ ID NO | Name |
|---|---|
| 1 | RECP04801 |
| 2 | RECP04667 |
| 3 | RECP03768 |
| 4 | RECP03742 |
| 5 | RECP01846 |
| 6 | RECP03559 |
| 7 | RECP04474 |
| 8 | RECP02965 |
| 9 | RECP00299 |
| 10 | RECP02979 |
| 11 | RECP00065 |
| 12 | RECP00145 |
| 13 | RECP00286 |
| 14 | RECP02936 |
| 15 | RECP04462 |
| 16 | RECP04479 |
| 17 | RECP01388 |
| 18 | RECP04480 |
| 19 | RECP00323 |
| 20 | RECP01887 |
| 21 | RECP01896 |
| 22 | RECP01897 |
| 23 | RECP01900 |
| 24 | RECP01903 |
| 25 | RECP01904 |
| 26 | RECP01907 |
| 27 | RECP02039 |
| 28 | RECP02042 |
| 29 | RECP02043 |
| 30 | RECP02338 |
| 31 | RECP04656 |
| 32 | RECP01901 |
| 33 | RECP02045 |
| 34 | RECP02046 |
| 35 | RECP03810 |
| 36 | RECP04478 |
| 37 | RECP00289 |
| 38 | RECP02782 |
| 39 | RECP03743 |
| 40 | RECP03745 |
| 41 | RECP04453 |
| 42 | RECP04456 |
| 43 | RECP04458 |
| 44 | RECP04496 |
| 45 | RECP04540 |
| 46 | RECP04705 |
| 47 | RECP03665 |
| 48 | RECP03771 |
| 49 | RECP04505 |
| 50 | RECP00271 |
| 51 | RECP00274 |
| 52 | RECP00318 |
| 53 | RECP01184 |
| 54 | RECP01306 |
| 55 | RECP01307 |
| 56 | RECP01398 |
| 57 | RECP02717 |
| 58 | RECP02774 |
| 59 | RECP02778 |
| 60 | RECP02929 |
| 61 | RECP02930 |
| 62 | RECP02931 |
| 63 | RECP02932 |
| 64 | RECP02933 |
| 65 | RECP02934 |
| 66 | RECP02935 |
| 67 | RECP02956 |
| 68 | RECP02963 |
| 69 | RECP03142 |
| 70 | RECP03144 |
| 71 | RECP03145 |
| 72 | RECP03146 |
| 73 | RECP03740 |
| 74 | RECP03747 |
| 75 | RECP03755 |
| 76 | RECP03756 |
| 77 | RECP03757 |
| 78 | RECP03758 |
| 79 | RECP03759 |
| 80 | RECP03760 |
| 81 | RECP03761 |
| 82 | RECP03764 |
| 83 | RECP03766 |
| 84 | RECP03776 |
| 85 | RECP04464 |
| 86 | RECP04575 |
| 87 | RECP04740 |
| 88 | RECP04742 |
| 89 | RECP00320 |
| 90 | RECP02779 |
| 91 | RECP02962 |
| 92 | RECP02993 |
| 93 | RECP03765 |
| 94 | RECP04686 |
| 95 | RECP02040 |
| 96 | RECP02195 |
| 97 | RECP02476 |
| 98 | RECP04460 |
| 99 | RECP03796 |
| 100 | RECP04046 |
| 101 | RECP00182 |
| 102 | RECP00955 |
| 103 | RECP03614 |
| 104 | RECP04758 |
| 105 | RECP00156 |
| 106 | RECP00821 |
| 107 | RECP01147 |
| 108 | RECP02564 |
| 109 | RECP03085 |
| 110 | RECP00198 |
| 111 | RECP00751 |
| 112 | RECP02152 |
| 113 | RECP02221 |
| 114 | RECP03591 |
| 115 | RECP02237 |
| 116 | RECP00167 |
| 117 | RECP02453 |
| 118 | RECP00092 |
| 119 | RECP00183 |
| 120 | RECP01092 |
| 121 | RECP02481 |
| 122 | RECP03191 |
| 123 | RECP03194 |
| 124 | RECP03399 |
| 125 | RECP00778 |
| 126 | RECP01607 |
| 127 | RECP01205 |
| 128 | RECP01576 |
| 129 | RECP00323 |
| 130 | RECP00752 |
| 131 | RECP00933 |
| 132 | RECP01276 |
| 133 | RECP01416 |
| 134 | RECP01834 |
| 135 | RECP02458 |
| 136 | RECP03533 |
| 137 | RECP03841 |
| 138 | RECP04121 |
| 139 | RECP04553 |
| 140 | RECP04625 |
| 141 | RECP01536 |
| 142 | RECP00086 |
| 143 | RECP00769 |
| 144 | RECP01006 |
| 145 | RECP01611 |
| 146 | RECP01750 |
| 147 | RECP02349 |
| 148 | RECP04188 |
| 149 | RECP00053 |
| 150 | RECP00054 |
| 151 | RECP00118 |
| 152 | RECP00535 |
| 153 | RECP00750 |
| 154 | RECP01673 |
| 155 | RECP02416 |
| 156 | RECP02876 |
| 157 | RECP03068 |
| 158 | RECP03340 |
| 159 | RECP03342 |
| 160 | RECP03556 |
| 161 | RECP04322 |
| 162 | RECP04606 |
| 163 | RECP00609 |
| 164 | RECP02100 |
| 165 | RECP02302 |
| 166 | RECP1398A |
| 167 | RECP04463 |
| 168 | RECP04475 |

### REFERENCES

### The Contents of which are Hereby Incorporated by Reference

[1] Russo & Johnson (2000) J Infect Dis 181:1753-1754.
[2] Uehling et al. (1997) J Urol 157:2049-2052.
[3] Tammen (1990) Br J Urol 65:6-9.
[4] Langermann et al. (1997) Science 276:607-611.
[5] WO03/074553.
[6] WO01/66572.
[7] Janke et al. (2001) FEMS Microbiol Lett 199:61-66.
[8] WO2004/005535.
[9] Dobrindt et al. (2002) Infect Immun 70:6365-6372.
[10] US2003/0165870.
[11] Welch et al. (2002) Proc Natl Acad Sci USA 99:17020-17024.
[12] American Type Culture Collection: ATCC 700928.
[13] European Journal of Biochemistry 2003; 1 Supplement 1 July: abstract P1.3-11.
[14] Geysen et al. (1984) PNAS USA 81:3998-4002.
[15] Carter (1994) Methods Mol Biol 36:207-223.
[16] Jameson, B A et al. 1988, CABIOS 4(1):181-186.
[17] Raddrizzani & Hammer (2000) Brief Bioinform 1(2):179-189.
[18] De Lalla et al. (1999) J. Immunol. 163:1725-1729.
[19] Brusic et al. (1998) Bioinformatics 14(2):121-130
[20] Meister et al. (1995) Vaccine 13(6):581-591.
[21] Roberts et al. (1996) AIDS Res Hum Retroviruses 12(7):593-610.
[22] Maksyutov & Zagrebelnaya (1993) Comput Appl Biosci 9(3):291-297.
[23] Feller & de la Cruz (1991) Nature 349(6311):720-721.
[24] Hopp (1993) Peptide Research 6:183-190.
[25] Welling et al. (1985) FEBS Lett. 188:215-218.
[26] Davenport et al. (1995) Immunogenetics 42:392-397.
[27] WO2006/091517.
[28] WO2006/089264.
[29] Bodanszky (1993) Principles of Peptide Synthesis (ISBN: 0387564314).
[30] Fields et al. (1997) Meth Enzymol 289: Solid-Phase Peptide Synthesis. ISBN: 0121821900.
[31] Chan & White (2000) Fmoc Solid Phase Peptide Synthesis. ISBN: 0199637245.
[32] Kullmann (1987) Enzymatic Peptide Synthesis. ISBN: 0849368413.
[33] Ibba (1996) Biotechnol Genet Eng Rev 13:197-216.
[34] Breedveld (2000) Lancet 355(9205):735-740.
[35] Gorman & Clark (1990) Semin. Immunol. 2:457-466.
[36] U.S. Pat. No. 5,707,829
[37] Current Protocols in Molecular Biology (F. M. Ausubel et al. eds., 1987) Supplement 30.
[38] EP-B-0509612.
[39] EP-B-0505012.
[40] Johnson & Steil (2001) J Clin Microbiol 39:3712-3717.
[41] Tang et al. (1997) Clin. Chem. 43:2021-2038.
[42] WO2006/046143
[43] Bernadac et al. (1998) J Bacteriol 180(18):4872-4878.
[44] EP-1441036.
[45] Sorensen & Mortensen (2005) Journal of Biotechnology 115:113-128.
[46] Meynial-Salles et al. (2005) Applied and Environmental Microbiology 71:2140-2144.
[47] US2004/0209370.
[48] WO00/68253.
[49] WO97/04110.
[50] Alper et al. (2005) Proc. Natl. Acad. Sci. USA 102:12678-12683.
[51] WO 01/09350.
[52] European patent 0624376.
[53] Vaccine Design . . . (1995) eds. Powell & Newman. ISBN: 030644867X. Plenum.
[54] WO00/23105.
[55] WO90/14837.
[56] Podda (2001) Vaccine 19:2673-2680.
[57] Frey et al. (2003) Vaccine 21:4234-4237.
[58] Vaccine Adjuvants: Preparation Methods and Research Protocols (Volume 42 of Methods in Molecular Medicine series). ISBN: 1-59259-083-7. Ed. O'Hagan.
[59] U.S. Pat. No. 6,299,884.
[60] U.S. Pat. No. 6,451,325.
[61] Allison & Byars (1992) Res Immunol 143:519-525.
[62] Hariharan et al. (1995) Cancer Res 55:3486-3489.
[63] U.S. Pat. No. 5,057,540.
[64] WO96/33739.
[65] EP-A-0109942.
[66] WO96/11711.
[67] WO00/07621.
[68] Barr et al. (1998) Advanced Drug Delivery Reviews 32:247-271.
[69] Sjolanderet et al. (1998) Advanced Drug Delivery Reviews 32:321-338.
[70] Niikura et al. (2002) Virology 293:273-280.
[71] Lenz et al. (2001) J Immunol 166:5346-5355.
[72] Pinto et al. (2003) J Infect Dis 188:327-338.
[73] Gerber et al. (2001) Virol 75:4752-4760.
[74] WO03/024480
[75] WO03/024481
[76] Gluck et al. (2002) Vaccine 20:B 10-B16.
[77] EP-A-0689454.
[78] Johnson et al. (1999) Bioorg Med Chem Lett 9:2273-2278.
[79] Evans et al. (2003) Expert Rev Vaccines 2:219-229.
[80] Meraldi et al. (2003) Vaccine 21:2485-2491.
[81] Pajak et al. (2003) Vaccine 21:836-842.
[82] Kandimalla et al. (2003) Nucleic Acids Research 31:2393-2400.
[83] WO02/26757.
[84] WO99/62923.
[85] Krieg (2003) Nature Medicine 9:831-835.
[86] McCluskie et al. (2002) FEMS Immunology and Medical Microbiology 32:179-185.
[87] WO98/40100.
[88] U.S. Pat. No. 6,207,646.
[89] U.S. Pat. No. 6,239,116.
[90] U.S. Pat. No. 6,429,199.
[91] Kandimalla et al. (2003) Biochemical Society Transactions 31 (part 3):654-658.
[92] Blackwell et al. (2003) J Immunol 170:4061-4068.
[93] Krieg (2002) Trends Immunol 23:64-65.
[94] WO01/95935.
[95] Kandimalla et al. (2003) BBRC 306:948-953.
[96] Bhagat et al. (2003) BBRC 300:853-861.
[97] WO03/035836.
[98] WO95/17211.
[99] WO98/42375.
[100] Beignon et al. (2002) Infect Immun 70:3012-3019.
[101] Pizza et al. (2001) Vaccine 19:2534-2541.
[102] Pizza et al. (2000) Int J Med Microbiol 290:455-461.
[103] Scharton-Kersten al. (2000) Infect Immun 68:5306-5313.
[104] Ryan et al. (1999) Infect Immun 67:6270-6280.
[105] Partidos et al. (1999) Immunol Lett 67:209-216.
[106] Peppoloni et al. (2003) Expert Rev Vaccines 2:285-293.
[107] Pine et al. (2002) J Control Release 85:263-270.
[108] Domenighini et al. (1995) Mol Microbiol 15:1165-1167.
[109] WO03/011223.
[110] WO99/40936.
[111] WO99/44636.
[112] Lillard J W et al., (2003) Blood 101(3):807-814. Epub 2002 September 12.
[113] Singh et al] (2001) J Cont Release 70:267-276.
[114] WO99/27960.
[115] U.S. Pat. No. 6,090,406
[116] U.S. Pat. No. 5,916,588
[117] EP-A-0626169.
[118] WO99/52549.
[119] WO01/21207.
[120] WO01/21152.
[121] Andrianov et al. (1998) Biomaterials 19:109-115.
[122] Payne et al. (1998) Adv Drug Delivery Review 31:185-196.
[123] U.S. Pat. No. 4,680,338.
[124] U.S. Pat. No. 4,988,815.
[125] WO92/15582.
[126] Stanley (2002) Clin Exp Dermatol 27:571-577.
[127] Wu et al. (2004) Antiviral Res. 64(2):79-83.
[128] Vasilakos et al. (2000) Cell Immunol. 204(1):64-74.
[129] U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.
[130] Jones (2003) Curr Opin Investig Drugs 4:214-218.
[131] WO04/60308
[132] WO04/64759.
[133] U.S. Pat. No. 6,924,271.
[134] US2005/0070556.
[135] U.S. Pat. No. 5,658,731.
[136] Wong et al. (2003) J Clin Pharmacol 43(7):735-42.
[137] US2005/0215517.
[138] WO02/072012.
[139] Signorelli & Hadden (2003) Int Immunopharmacol 3(8):1177-1186.
[140] WO2004/064715.
[141] Cooper (1995) Pharm Biotechnol 6:559-580.
[142] WO2006/002422
[143] WO2004/87153.
[144] U.S. Pat. No. 6,605,617.
[145] WO02/18383.
[146] WO2004/018455.
[147] WO03/082272.
[148] U.S. Pat. No. 5,011,828.
[149] U.S. Pat. No. 6,586,409.
[150] WO99/11241.
[151] WO94/00153.
[152] WO98/57659.
[153] European patent applications 0835318, 0735898 and 0761231.
[154] WO03/009869.
[155] Donnelly et al. (1997) Annu Rev Immunol 15:617-648.
[156] Strugnell et al. (1997) Immunol Cell Biol 75(4):364-369.
[157] Cui (2005) Adv Genet. 54:257-89.
[158] Robinson & Torres (1997) Seminars in Immunol 9:271-283.
[159] Brunham et al. (2000) J Infect Dis 181 Suppl 3:S538-S543.
[160] Svanholm et al. (2000) Scand J Immunol 51(4):345-353.
[161] DNA Vaccination—Genetic Vaccination (1998) eds. Koprowski et al. (ISBN 3540633928).
[162] Gene Vaccination: Theory and Practice (1998) ed. Raz (ISBN 3540644288).
[163] Findeis et al., Trends Biotechnol. (1993) 11:202.
[164] Chiou et al. (1994) Gene Therapeutics: Methods And Applications Of Direct Gene Transfer. ed. Wolff.
[165] Wu et al., J. Biol. Chem. (1988) 263:621.
[166] Wu et al., J. Biol. Chem. (1994) 269:542.
[167] Zenke et al., Proc. Natl. Acad. Sci. (USA) (1990) 87:3655.
[168] Wu et al., J. Biol. Chem. (1991) 266:338.
[169] Jolly, Cancer Gene Therapy (1994)1:51.
[170] Kimura, Human Gene Therapy (1994) 5:845.
[171] Connelly, Human Gene Therapy (1995) 1:185.
[172] Kaplitt, Nature Genetics (1994) 6:148.
[173] WO 90/07936.
[174] WO 94/03622.
[175] WO 93/25698.
[176] WO 93/25234.
[177] U.S. Pat. No. 5,219,740.
[178] WO 93/11230.
[179] WO 93/10218.
[180] U.S. Pat. No. 4,777,127.
[181] GB 2,200,651.
[182] EP-A-0 345 242.
[183] WO 91/02805.
[184] WO 94/12649.
[185] WO 93/03769.
[186] WO 93/19191.
[187] WO 94/28938.
[188] WO 95/11984.
[189] WO 95/00655.
[190] Curiel, Hum. Gene Ther. (1992) 3:147.
[191] Wu, J. Biol. Chem. (1989) 264:16985.
[192] U.S. Pat. No. 5,814,482.
[193] WO 95/07994.
[194] WO 96/17072.
[195] WO 95/30763.
[196] WO 97/42338.
[197] WO 90/11092.
[198] U.S. Pat. No. 5,580,859.
[199] U.S. Pat. No. 5,422,120.
[200] WO 95/13796.
[201] WO 94/23697.
[202] WO 91/14445.
[203] EP 0524968.
[204] Philip, Mol. Cell. Biol. (1994)14:2411.
[205] Woffendin, Proc. Natl. Acad. Sci. (1994) 91:11581.
[206] U.S. Pat. No. 5,206,152.
[207] WO 92/11033.
[208] U.S. Pat. No. 5,149,655.
[209] Glezen & Alpers (1999) Clin. Infect. Dis. 28:219-224.
[210] Johnson et al. (2001) Infect Immun 69:1306-1314.
[211] Johnson et al. (2001) J Infect Dis 183:897-906 (see also 183:1546).
[212] Johnson, Hopkins (1998) Infection and Immunity 66:6063-6064.
[213] Bahrani-Mougeot et al.; Molecular Microbiology (2002) 45(4), 1079-1093
[214] Johnson et al., Infection and Immunity, (1998) 3059-3065
[215] Almeida & Alpar (1996) J. Drug Targeting 3:455-467.
[216] Agarwal & Mishra (1999) Indian J Exp Biol 37:6-16.
[217] Costantino et al. (1992) Vaccine 10:691-698.
[218] Costantino et al. (1999) Vaccine 17:1251-1263.
[219] International patent application WO03/007985.
[220] WO 99/24578.
[221] WO 99/36544.
[222] WO 99/57280.
[223] WO 00/66791.
[224] WO 01/64922.
[225] WO 01/64920.
[226] WO 03/020756.
[227] WO 2004/032958.
[228] WO 2004/048404.
[229] Watson (2000) Pediatr Infect Dis J 19:331-332.
[230] Rubin (2000) Pediatr Clin North Am 47:269-285.
[231] Jedrzejas (2001) Microbiol Mol Biol Rev 65:187-207.
[232] Bell (2000) Pediatr Infect Dis J19:1187-1188.
[233] Iwarson (1995) APMIS103:321-326.
[234] Gerlich et al. (1990) Vaccine 8 Suppl:S63-68 & 79-80.
[235] Hsu et al. (1999) Clin Liver Dis 3:901-915.
[236] Stratov et al. (2004) Curr Drug Tgts 5(1):71-88.
[237] Plotkin et al. (eds) (2003) Vaccines, 4th edition (W.B. Saunders Company)
[238] Del Guidice et al. (1998) Molecular Aspects of Medicine 19:1-70.
[239] Gustafsson et al. (1996) N. Engl. J. Med. 334:349-355.
[240] Rappuoli et al. (1991) TIBTECH 9:232-238.
[241] Sutter et al. (2000) Pediatr Clin North Am 47:287-308.
[242] Zimmerman & Spann (1999) Am Fam Physician 59:113-118, 125-126.
[243] McMichael (2000) Vaccine 19 Suppl 1:S101-S107.
[244] Schuchat (1999) Lancet 353(9146):51-56.
[245] WO02/34771.
[246] WO 99/54457.
[247] WO 04/01846.
[248] WO 04/041157.
[249] WO 05/028618.
[250] WO 02/079243.
[251] WO 02/02606.
[252] Kalman et al. (1999) Nature Genetics 21:385-389.
[253] Read et al. (2000) Nucleic Acids Res 28:1397-1406.
[254] Shirai et al. (2000) J. Infect. Dis. 181(Suppl 3):S524-S527.
[255] WO 99/27105.
[256] WO 00/27994.
[257] WO 00/37494.
[258] WO2005/084306.
[259] WO2005/002619.
[260] Ross et al. (2001) Vaccine 19:4135-4142.
[261] Dreesen (1997) Vaccine 15 Supp1:S2-S6.
[262] MMWR Morb Mortal Wkly Rep 1998 Jan. 16; 47(1):12, 19.
[263] Anderson (2000) Vaccine 19 Suppl 1:S59-S65.
[264] Kahn (2000) Curr Opin Pediatr 12:257-262.
[265] Crowe (1995) Vaccine 13:415-421.
[266] Modlin et al. (2001) J Toxicol Clin Toxicol 39:85-100.
[267] Demicheli et al. (1998) Vaccine 16:880-884.
[268] Stepanov et al. (1996) J Biotechnol 44:155-160.
[269] Dale (1999) Infect Dis Clin North Am 13:227-43, viii.
[270] Ferretti et al. (2001) PNAS USA 98: 4658-4663.
[271] Kuroda et al. (2001) Lancet 357(9264):1225-1240; see also pages 1218-1219.
[272] WO 00/09699.
[273] WO 05/002619
[274] WO 05/084306
[275] EP-A-0372501
[276] EP-A-0378881
[277] EP-A-0427347
[278] WO93/17712
[279] WO94/03208
[280] WO98/58668
[281] EP-A-0471177
[282] EP-A-0594610.
[283] WO00/56360
[284] WO91/01146
[285] WO00/61761
[286] WO01/72337
[287] Falugi et al. (2001) Eur J Immunol 31:3816-3824.
[288] Baraldo et al, (2004) Infect Immun.72:4884-4887
[289] WO02/091998.
[290] Kuo et al. (1995) Infect Immun 63:2706-2713.
[291] Research Disclosure, 453077 (January 2002)
[292] Needleman& Wunsch (1970) J. Mol. Biol. 48, 443-453.
[293] Rice et al. (2000) Trends Genet. 16:276-277.
[294] Gennaro (2000) Remington: The Science and Practice of Pharmacy. 20th edition, ISBN: 0683306472.
[295] Methods In Enzymology (S. Colowick and N. Kaplan, eds., Academic Press, Inc.)
[296] Handbook of Experimental Immunology, Vols. I-IV (D. M. Weir and C. C. Blackwell, eds, 1986, Blackwell Scientific Publications)
[297] Sambrook et al. (2001) Molecular Cloning: A Laboratory Manual, 3rd edition (Cold Spring Harbor Laboratory Press).
[298] Handbook of Surface and Colloidal Chemistry (Birdi, K. S. ed., CRC Press, 1997)
[299] Ausubel et al. (eds) (2002) Short protocols in molecular biology, 5th edition (Current Protocols).
[300] Molecular Biology Techniques: An Intensive Laboratory Course, (Ream et al., eds., 1998, Academic Press)
[301] PCR (Introduction to Biotechniques Series), 2nd ed. (Newton & Graham eds., 1997, Springer Verlag)
[302] Murphy (1998) J. Bacteriol 180:2063-2071.

1: An isolated polypeptide comprising: an amino acid sequence (a) comprising SEQ ID NO: 80; (b) having at least 80% sequence identity to SEQ ID NO: 80; (c) which comprises at least 10 consecutive amino acids of SEQ ID NO: 80; or (d) having at least 80% sequence identity to SEQ ID NO: 80 and including at least 10 consecutive amino acids of SEQ ID NO: 80.

2: The polypeptide of claim 1, wherein said fragment comprises at least one B-cell epitope of (a).

3: A pharmaceutical composition comprising the polypeptide of claim 1 in admixture with a pharmaceutically acceptable carrier.

4: A pharmaceutical composition comprising two or more polypeptides of claim 1 in admixture with a pharmaceutically acceptable carrier.

5: The composition of claim 3, further comprising a vaccine adjuvant.

6: An immunogenic composition comprising one or more outer membrane vesicles (OMVs) expressing one or more polypeptides comprising: an amino acid sequence (a) comprising SEQ ID NO: 80; (b) having at least 80% sequence identity to SEQ ID NO: 80; (c) which comprises at least 10 consecutive amino acids of SEQ ID NO: 80; or (d) having at least 80% sequence identity to SEQ ID NO: 80 and including at least 10 consecutive amino acids of SEQ ID NO: 80.

7: The immunogenic composition of claim 6, wherein said fragment comprises at least one B-cell epitope of (a).

8: A method for raising an immune response in a patient, comprising the step of administering to the patient a pharmaceutical composition comprising a pharmaceutically acceptable carrier in admixture with a polypeptide comprising an amino acid sequence (a) comprising SEQ ID NO: 80; (b) having at least 80% sequence identity to SEQ ID NO: 80; (c) which comprises at least 10 consecutive amino acids of SEQ ID NO: 80; or (d) having at least 80% sequence identity to SEQ ID NO: 80 and including at least 10 consecutive amino acids of SEQ ID NO: 80.

9: The method claim 8, wherein said polypeptide comprises at least one B-cell epitope of (a).

10: The method of claim 8, wherein the pharmaceutical composition further comprises a vaccine adjuvant.

11: The method of claim 8, wherein the polypeptide is expressed in an outer membrane vesicle.

12: The method of claim 8, wherein the amino acid sequence has at least 90% sequence identity to SEQ ID NO: 80.

13: The method of claim 8, wherein the polypeptide comprises the amino acid sequence set forth in SEQ ID NO: 80.

14: The method of claim 8, wherein the amino acid sequence has at least 20 consecutive amino acids of SEQ ID NO: 80.

15: The method of claim 8, wherein the amino acid sequence has at least 50 consecutive amino acids of SEQ ID NO: 80.

16: The method of claim 8, wherein the amino acid sequence has at least 90% sequence identity to SEQ ID NO: 80 and includes at least 20 consecutive amino acids of SEQ ID NO: 80.

17: The method of claim 8, wherein the amino acid sequence has at least 95% sequence identity to SEQ ID NO: 80 and includes at least 50 consecutive amino acids of SEQ ID NO: 80.


SEQUENCE LISTING (168 sequences)

SEQ ID NO: 1 - protein, 71 aa, Escherichia coli
MTMKLIKTLVAVSALSMMSFGVFAQSVSATASTLDRAEAKIAAQAAEQGASYKITSARVENRVYMTAELLK

SEQ ID NO: 2 - protein, 274 aa, Escherichia coli
VVNGHTDVIGSTSIRHILAVRQSTLLQIDTLIRQLAEISAMTVSIGGKTALDWAMKQDFRCGFWLMEKPEIAMKAITRNLDRELWRDLMQRSGMLSLMDAQARETWYRSLEYDNVPEISEANILNTFKQLHQNKDEVFERGVINVFRGLNWNYKTNLPCKFGSKIIVNNLVRWDRWGFHIVTGQQADRVADLERMLHLFSGWPIPDNRENIIIRLDDHIQSAQGQECYEYEDEMFSIRYFKKGSAHITFRKPELIDRLNDIIAKYYPEIIPYNI

SEQ ID NO: 3 - protein, 1024 aa, Escherichia coli
MPTITTAQIKSTLQSAKQSAANKLHSAGQSTKDALKKAAEQTRNAGNRLILLIPKDYKGQGSSLNDLVRTADELGIEVQYDEKNGTAITKQVFGTAEKLIGLTERGVTIFAPQLDKLLQKYQKAGNKLGGSAENIGDNLGKAGSVLSTFQNFLGTALSSMKIDELIKKQKSGSNVSSSELAKASIELINQLVDTAASINNNVNSFSQQLNKLGSVLSNTKHLNGVGNKLQNLPNLDNIGAGLDTVSGILSVISASFILSNADADTGTKAAAGVELTTKVLGNVGKGISQYIIAQRAAQGLSTSAAAAGLIASAVTLAISPLSFLSIADKFKRANKIEEYSQRFKKLGYDGDSLLAAFHKETGAIDASLTTISTVLASVSSGISAAATTSLVGAPVSALVGAVTGIISGILEASKQAMFEHVASKMADVIAEWEKKHGKNYFENGYDARHAAFLEDNFKILSQYNKEYSVERSVLITQQHWDTLIGELAGVTRNGDKTLSGKSYIDYYEEGKRLEKKPDEFQKQVFDPLKGNIDLSDSKSSTLLKFVTPLLTPGEEIRERRQSGKYEYITELLVKGVDKWTVKGVQDKGSVYDYSNLIQHASVGNNQYREIRIESHLGDGDDKVFLAAGSANIYAGKGHDVVYYDKTDTGYLTIDGTKATEAGNYTVTRVLGGDVKVLQEVVKEQEVSVGKRTEKTQYRSYEFTHINGTDLTETDNLYSVEELIGTNRADKFFGSKFTDIFHGADGDDHIEGNDGNDRLYGDKGNDTLRGGNGDDQLYGGDGNDKLTGGVGNNYLNGGDGDDELQVQGNSLAKNVLSGGKGNDKLYGSEGADLLDGGEGNDLLKGGYGNDIYRYLSGYGHHIIDDDGGKDDKLSLADIDFRDVAFKREGNDLIMYKAEGNVLSIGHKNGITFRNWFEKESGDISNHQIEQIFDKDGRVITPDSLKKAFEYQQSNNQANYVYGEYASTYADLDNLNPLINEISKIISAAGNFDVKEERSAASLLQLSGNASDFSYGRNSITLTASA

SEQ ID NO: 4 - protein, 409 aa, Escherichia coli
MELIMPLSRRNFIQNAVLGISAAGLSAAPALAKNISSSTAHIISKTSGHADTSTSKSLHIISLDRLEASAKDVMTEAAYAYIAHGAGDEWTYHENRRAFSDYPLLPHRLSGVAAHSIDIRTDLLGHHLEHPLLIAPMGAHMFVHPEGEVIAAAGAEKAGALYESSGASNRSLEDIAKASKGPKWFQLYFNADAGVTRSLLERAKAAGYSAIIITADALGPGTSDAFLSMSSPFPAGATFGNHDPRYGGKGDFFNQKVELTPADIEFVKKITGLPVIVKGILRGEDAVVAIDAGADAIQVSNHGGRQIDGVPSAISQLQEVAARVGHKVPVIFDSGIRRGIDVVRAISLGATAVAVGRPVLYGIAAGGVGGVASVIEHLKTELRTAMLLSGARTLKDLSQGFIRNKETEH

SEQ ID NO: 5 - protein, 555 aa, Escherichia coli
MAQVINTNSLSLITQNNINKNQSALSSSIERLSSGLRINSAKDDAAGQAIANRFTSNIKGLTQAARNANDGISVAQTTEGALSEINNNLQRIRELTVQASTGTNSDSDLDSIQDEIKSRLDEIDRVSGQTQFNGVNVLAKDGSMKIQVGANDGQTITIDLKKIDSDTLGLSGFNVNGKGAVANTAATKDDLVAASVSAAVGNEYTVSAGLSKSTAADVIASLTDGATVTAAGVSNGFAAGATGNAYKFNQANNTFTYNTTSTAAELQSYLTPKAGDTATFSVEIGSTKQDVVLASDGKITAKDGSKLYIDTTGNLTQNGGGTLEEATLNGLAFNHSGPAAAVQSTITTADGTSIVLAGSGDFGTTKTAGAINVTGAVISADALLSASKATGFTSGAYTVGTDGVVKSGGNDVYNKADGTGLTTDNTTKYYLQDDGSVTNGSGKAVYVDATGKLTTDAETKAATTADPLKALDEAISSIDKFRSSLGAVQNRLDSAVTNLNNTTTNLSEAQSRIQDADYATEVSNMSKAQIIQQAGNSVLAKANQVPQQVLSLLQG

SEQ ID NO: 6 - protein, 660 aa, Escherichia coli
MSRPQFTSLRLSLLALAVSATLPTFAFATETMTVTATGNARSSFEAPMMVSVIDTSAPENQTATSATDLLRYVPGITLDGTGRTNGQDVNMRGYDHRGVLVLVDGVRQGTDTGHLNGTFLDPALIKRVEIVRGPSALLYGSGALGGVISYDTVDAKDLLQEGQSSGFRVFGTGGTGDHSLGLGASAFGRTENLDGIVAWSSRDRGDLRQSNGETAPNDESINNMLAKGTWQIDSAQSLSGLVRYYNNDAREPKNPQTVEASDSSNPMVDRSTIQRDAQLSYKLAPQGNDWLNADAKIYWSEVRINAQNTGSSGEYREQITKGARLENRSTLFADSFASHLLTYGGEYYRQEQHPGGATTGFPQAKIDFSSGWLQDEITLRDLPITLLGGTRYDSYRGSSDGYKDVDADKWSSRAGMTINPTNWLMLFGSYAQAFRAPTMGEMYNDSKHFSIGRFYTNYWVPNPNLRPETNETQEYGFGLRFDDLMLSNDALEFKASYFDTKAKDYISTTVDFAAATTMSYNVPNAKIWGWDVMTKYTTDLFSLDVAYNRTRGKDTDTGEYISSINPDTVTSTLNIPIAHSGFSVGWVGTFADRSTHISSSYSKQPGYGVNDFYVSYQGQQALKGMTTTLVLGNAFDKEYWSPQGIPQDGRNGKIFVSYQW

SEQ ID NO: 7 - protein, 1024 aa, Escherichia coli
MPTITTAQIKSTLQSAKQSAANKLHSAGQSTKDALKKAAEQTRNAGNRLILLIPKDYKGQGSSLNDLVRTADELGIEVQYDEKNGTAITKQVFGTAEKLIGLTERGVTIFAPQLDKLLQKYQKAGNKLGGSAENIGDNLGKAGSVLSTFQNFLGTALSSMKIDELIKKQKSGGNVSSSELAKASIELINQLVDTAASLNNNVNSFSQQLNKLGSVLSNTKHLNGVGNKLQNLPNLDNIGAGLDTVSGILSAISASFILSNADADTGTKAAAGVELTTKVLGNVGKGISQYIIAQRAAQGLSTSAAAAGLIASVVTLAISPLSFLSIADKFKRANKIEEYSQRFKKLGYDGDSLLAAFHKETGAIDASLTTISTVLASVSSGISAAATTSLVGAPVSALVGAVTGIISGILEASKQAMFEHVASKMADVIAEWEKKHGKNYFENGYDARHAAFLEDNFEILSQYNKEYSVERSVLITQQHWDTLIGELAGVTRNGDKTLSGKSYIDYYEEGKRLEKEPDEFQKQVFDPLKGNIDLSVIKSSTLLKFITPLLTPGKEIRERRQSGKYEYITELLVKGVDKWTVKGVQDKGSVYDYSNLIQHASVGNNQYREIRIESHLGDGDDKVFLSAGSANIYAGKGHDVVYYDKTDTGYLTIDGTKATEAGNYTVTRVLGGDVKVLQEVVKEQEVSVGKRTEKTQYRSYEFTHINGTDLTETDNLYSVEELIGTNRADKFFGSKFTDIFHGADGDDHIEGNDGNDRLYGDKGNDTLRGGNGDDQLYGGDGNDKLTGGVGNNYLNGGDGDDELQVQGNSLAKNVLSGGKGNDKLYGSEGADLLDGGEGNDLLKGGYGNDIYRYLSGYGHHIIDDDGGKDDKLSLADIDFRDVAFKREGNDLIMYKAEGNVLSIGHKNGITFRNWFEKESGDISNHQIEQIFDKDGRVITPDSLKKAFEYQQSNNQANYVYGEYASTYADLDNLNPLINEISKIISAAGNFDVKEERSAASLLQLSGNASDFSYGRNSITLTASA

SEQ ID NO: 8 - protein, 948 aa, Escherichia coli
MKRHLNTSYRLVWNHITGTLVVASELARSRGKRAGVAVALSLAAVTSVPALAADTVVQAGETVNDGTLTNHDNQIVLGTANGMTISTGLEYGPDNEANTGGQWIQNGGIANNTTVTGGGLQRVNAGGSVSDTVISAGGGQSLQGQAVNTTLNGGEQWVHEGGIATGTVINEKGWQAVKSGAMATDTVVNTGAEGGPDAENGDTGQFVRGNAVRTTINENGRQIVAAEGTANTTVVYAGGDQTVHGYALDTTLNGGNQYVHNGGTASGTVVNSDGWQIVKEGGLADFTIVNQKGKLQVNAGGTATNVTLKQGGALVTSTAATVTGSNRLGNFTVENGNADGVVLESGGRLDVLEGHSAWKTLVDDGGTLAVSAGGKATDVTMTSGGALIADSGATVEGTNASGKFSIDGISGQASGLLLENGGSFTVNAGGQAGNTTVGHRGTLTLAAGGSLSGRTQLSKGASMVLNGDVVSTGDIVNAGEIHFDNQTTPDAALSRAVAKGDSPVTFHKLTTSNLTGQGGTINMRVRLDGSNTSDQLVINGGQATGKTWLAFTNVGNSNLGVATTGQGIRVVDAQNGATTEEGAFALSRPLQAGAFNYTLNRDSDEDWYLRSENAYRAEVPLYASMLTQAMDYDRILAGSRSHQTGVNGENNSFRLSIQGGHLGHVNNGGIARGATPESSGSYGLVRLEGDLLRTEVAGMSLTTGVYGAAGHSSVDVKDDDGSRAGTVRDDAGSLGGYLNLVHTSSGLWADIVAQGTRHSMKASSDNNDFRARGWGWLGSLETGLPFSITDNLMLEPQLQYTWQGLSLDDGQDNAGYVKFGHGSAQHVRAGFRLGSHNDMNFGKGTSSRDTLRDSAKHSVRELPVNWWVQPSVIRTFSSRGDMSMGTAAAGSNMTFSPSQNGTTLDLQAGLEARVRENITLGVQAGYAHSVSGSSAEGYNGQATLNVTF

SEQ ID NO: 9 - protein, 725 aa, Escherichia coli
MRINKILWSLTVLLVGLNSQVSVAKYSDDDNDETLVVEATAEQVLKQQPGVSVITSEDIKKTPPVNDLSDIIRKMPGVNLTGNSASGTRGNNRQIDIRGMGPENTLILIDGVPVTSRNSVRYSWRGERDTRGDTNWVPPEQVERIEVIRGPAAARYGSGAAGGVVNIITKRPTNDWHGSLSLYTNQPESSEEGATRRANFSLSGPLAGDALTTRLYGNLNKTDADSWDINSPVGTKNAAGHEGVRNKDINGVISWKLNPQQILDFEAGYSRQGNIYAGDTQNSSSSAVTESLAKSGKETNRLYRQNYGITHNGIWDWGQSRFGIYYEKTNNTRMNEGLSGGGEGRILAGEKFTTNRLSSWRTSGELNIPLNVMVDQTLTVGAEWNRDKLDDPSSTSLTVNDRDISGISGSAADRSSKNHSQISALYIEDNIEPVPGTNIIPGLRFDYLSDSGGNFSPSLNLSQELGDYFKVKAGVARTFKAPNLYQSSEGYLLYSKGNGCPKDITSGGCYLIGNKDLDPEISVNKEIGLEFTWEDYHASVTYFRNDYQNKIVAGDNVIGQTASGAYILKWQNGGKALVDGIEASMSFPLVKDRLNWNTNATWMITSEQKDTGNPLSVIPKYTINNSLNWTITQAFSANVNWTLYGRQKPRTHAETRSEDTGGLSGKELGAYSLVGTNFNYDINKNLRLNVGVSNILNKQIFRSSEGANTYNEPGRAYYAGVTASF

SEQ ID NO: 10 - protein, 558 aa, Escherichia coli
MKLFKSILLIAACHAAQASAAIDINADPNLTGAAPLTGILNGQQSDTQNMSGFDNTPPPAPPVVMSRMFGAQLFNGTSADSGATVGFNPDYILNPGDSIQVRLWGAFTFDGALQIDPKGNIFLPNVGPVKVAGVSNSQLNTLVTSKVKEVYQSNVNVYASLLQAQPVKVYVTGFVRNPGLYGGVTSDSLLNYLIKAGGVDPERGSYVDIVVKRGNRVRSNVNLYDFLLNGKLGLSQFADGDTIIVGPRQHTFSVQGDVFNSYDFEFRESSIPVTEALSWARPKPGATHITIMRKQGLQKRSEYYPISSAPGRMLQNGDTLIVSTDRYAGTIQVRVEGAHSGEHAMVLPYGSTMRAVLEKVRPNSMSQMNAVQLYRPSVAQRQKEMLNLSLQKLEEASLSAQSSTKEEASLRMQEAQLISRFVAKARTVVPKGEVILNESNIDSVLLEDGDVINIPEKTSLVMVHGEVLFPNAVSWQKGMTTEDYIEKCGGLTQKSGNARIIVIRQNGAAVNAEDVDSLKPGDEIMVLPKYESKNIEVTRGISTILYQLAVGAKVILSL

SEQ ID NO: 11 - protein, 140 aa, Escherichia coli
MSKYIYILLSFLVLFFIFFYAYISLMSKEHHYTQHELSPFFLYTPESLRNLPNISNVAEYSYYYNVDDMQTRVIVTWRNIDNIFLQKAKLIDFLKRMGPSLQNDCIWFFHDKSDYANNFQRYCIIEHRDSLQVEYFETIE

SEQ ID NO: 12 - protein, 188 aa, Escherichia coli
MIKITPHKITILMGLLLSPSVFATDVNVDFTATVKATTCNITLTGTNVTDNGNDKYTLVIPSMGMDKIANKTAQSEANFKLVANGCSSGISWIDTTLTGNQSGSSPALIIPLASDTTSTTSYIGMGFKRKATSGDTFLKPNSAEYIRWSASEISTDGLEMTVALRETSVGKGVPGKFRALATFNFSYQ

SEQ ID NO: 13 - protein, 149 aa, Escherichia coli
VDHWPNLLAFHFTLYSAEGNINGQQIHAFCTAFYRQVQEHITERNHTASPAPPVVLRWLREQHGGATIRCLLLLSQASICHLRVSATVDEECSQVVDLLQQAWRGINAGGQCRVERCFRVTWPDTSEQYVALKTAVQSLIPLVIATIIR

SEQ ID NO: 14 - protein, 164 aa, Escherichia coli
VTLNTSQVSYYITQRKKGITQHISAMKAGISVRSGRRIEKGQRAKNSVRHWSTRKDPLEAVWDSMLVPLLKERPVLTPTTLLEMLQDKYPGQYPNSFRRTMQRRGCEWKLQSGAEQEVMFRQWHQPGLRGLLDFTKLKGVVVTIAGKLLVHMLYNFRLEWSHWS

SEQ ID NO: 15 - protein, 840 aa, Escherichia coli
VMRVMKDRIPFAVNNITCVILLSLFCNAASAVEFNTDVLDAADKKNIDFTRFSEAGYVLPGQYLLDVIVNGQSISPASLQISFVEPQSSGDKAEKKLPQACLTSDMVRLMGLTAESLDKVVYWHDGQCADFHGLPGVDIRPDTGAGVLRINMPQAWLEYSDATWLPPSRWDDGIPGLMLDYNLNGTVSRNYQGGDSHQFSYNGTVGGNLGPWRLRADYQGSQEQSRYNGEKTTNRNFTWSRFYLFRAIPRWRANLTLGENNINSDIFRSWSYTGASLESDDRMLPPRLRGYAPQITGIAETNARVVVSQQGRVLYDSMVPAGPFSIQDLDSSVRGRLDVEVIEQNGRKKTFQVDTASVPYLTRPGQVRYKLVSGRSRGYGHETEGPVFATGEASWGLSNQWSLYGGAVLAGDYNALAAGAGWDLGVPGTLSADITQSVARIEGERTFQGKSWRLSYSKRFDNADADITFAGYRFSERNYMTMEQYLNARYRNDYSSREKEMYTVTLNKNVADWNTSFNLQYSRQTYWDIRKTDYYTVSVNRYFNVFGLQGVAVGLSASRSKYLGRDNDSAYLRISVPLGTGTASYSGSMSNDRYVNMAGYTDTFNDGLDSYSLNAGLNSGGGLTSQRQINAYYSHRSPLANLSANIASLQKGYTSFGVSASGGATITGKGAALHAGGMSGGTRLLVDTDGVGGVPVDGGQVVTNRWGTGVVTDISSYYRNTTSVDLKRLPDDVEATRSVVESALTEGAIGYRKFSVLKGKRLFAILRLADGSQPPFGASVTSEKGRELGMVADEGLAWLSGVTPGETLSVNWDGKIQCQVNVPETAISDQQLLLPCTPQK

SEQ ID NO: 16 - protein, 239 aa, Escherichia coli
MNNVKKILLMEDDYDIAALLRLNLQDEGYQIVHEADGARARLLLDKQTWDAVILDLMLPNVNGLEICRYIRQMTSYLPVIIISARTSETHRVLGLEMGADDYLPKPFSIPELIARIKALFRRQEAMGQNILLASGLICCHGLCINPFSREVHLHNKQVDLTPREFDLLLWFARHPGEVFSRLSLLDNVWGYQHEGYEHTVNTHINRLRAKIEQDAAEPKMIQTVWGKGYRFSVDNAGMR

SEQ ID NO: 17 - protein, 122 aa, Escherichia coli
MNYALIIAIIISFPCALLSVFLVLKGWALIGDAMNHAMFPGIVLAWIIGMPLGIGAFIVGLSCAIATGYLKDNSIIKKDTIIGIVFSGIFVLTINRHKSADYRYHYYITVYSAWYRLPFLPH

SEQ ID NO: 18 - protein, 283 aa, Escherichia coli
MNCSLTLSQRLSLVFTVVLLFCAAVTCGVHIYSSNLYGNAMVQRLSAGLAQQIVITEPLLDNRGQVNHRTLKGLFERLMTLNPSVELYIVSPEGRLLVEAAPPGHIKRRYINIAPLKKFLSGAVWPVYGDDPRSVNKKKVFSTAPLYLRDDLKGYLYIILQGEELNALTDAAWTKALWNALYWSLFLVVICGLLSGMLVWYWVTRPIQQLTENVSGIEQDSISAIKQLAIQRPATPLATRSRYYTMPSLNWPVKYPVSGINFQKVINSAVNLLPISPMIYGRH

SEQ ID NO: 19 - protein, 1042 aa, Escherichia coli
MLMKRHLNTCYRLVWNHITGAFVVASELARARGKRGGVAVALSLAAVTSLPVLAADIVVHPGETVNGGTLVNHDNQFVSGTADGVTVSTGLELGPDSDENTGGQWIKAGGTGRNTTVTANGRQIVQAGGTASDTVIRDGGGQSLNGLAVNTTLDNRGEQWVHGGGKAAGTIINQDGYQTIKHGGLATGTIVNTGAEGGPESENVSTGQMVGGTAESTTINNNGRQVIWSSGVSRDTLIYTGGDQTVHGEAHNTRLEGGNQYVHKYGLALNTVINEGGWQVVKAGGTAGNTTINQNGELKVHAGGEASDVTQNTGGALVTSTAATVTGTNRLGAFSVVEGKADNVVLENGGRLDVLSGHTATNTRVDDGGTLDVRNGGTATTVSMGNGGVLLADSGAAVSGTRSDGTAFRIGGGQADALMLEKGSSFTLNAGDTATDTTVNGGLFTARGGSLAGTTTLNNGAILTLSGKTVNNDTLTIREGDALLQGGSLTGNGSVEKSGSGTLTVSNTTLTQKAVNLNEGTLTLNDSTVTTDVIAQRGTALKLTGSTVLNGAIDPTNVTLASGATWNIPDNATVQSVVDDLSHAGQIHFTSTRTGKFVPATLKVKNLNGQNGTISLRVRPDMAQNNADRLVIDGGRATGKTILNLVNAGNSASGLATSGKGIQVVEAINGATTEEGAFVQGNRLQAGAFNYSLNRDSDESWYLRSENAYRAEVPLYASMLTQAMDYDRILAGSRSHQTGVNVKNNSVRLSIQGGHLGHDNNGGIARGATPESSGSYGFVRLEGDLLRTEVAGMSVTAGVYGAAGHSSVDVKDDDGSRAGTVRDDAGSLGGYLNLTHTSSGLWADIVAQGTRHSMKASSDNNDFRARGWGWLGSLETGLPFSITDNLMLEPQLQYTWQGLSLDDGQDNAGYVKFGHGSAQHVRAGFRLGSHSDMTFGEGTSSRDTLRDSAKHSVRELPVNWWVQPSVIRTFSSRGDMSMGTAAAGSNMTFSPSQNGTSLDLQAGLEARVRENITLGVQAGYAHSVSGSSAEGYNGQATLNVTF

SEQ ID NO: 20 - protein, 352 aa, Escherichia coli
MKRKVLAMLVPALLVAGAANAAEIYNKNGNKVELYGKMVGERILTDRESGEKGDNSQDTSYARVGVKGETQINPELTGYGQFELDLEASNRHNPDQTRLAYAGLSYKDFGSFDYGRNVGVAYDAEAFTDMFVEWGGDSWAGTDLFMTNRTNGVATYRNTDFFGMVEGLNFALQYQGKNEGTGNYKANGDGHGLSATYTIDGFSFAGAYANSDRTDWQSGDGKGERAEVWALSTKYDANNVYAAVMYGESHNMNSDDGDVVNKTQNFEAVLQYQFDFGLRPSIGYSYSKALDVAGYKDSDRLNYIEIGTWYYFNKNMNVYTAYQINLLDKSDYVLAHGLNTDDQLAVGIVYQF

SEQ ID NO: 21 - protein, 487 aa, Escherichia coli
MIKKKGFTLLEVTIVLGIGTLIAFMKFQDMRNDQEAVLADNVGTQIKQLGEAVNRYISIRYDKISTLSSSNNQSSDPGPRTCSAAGCEITYQTLINEGLLPVGYTGTNAQKASYKIVLKRSGTAPDYVINGLITTASPWEEGGRIRYDLLGKAVQAAGVDGGMSRNTKIASGYGGQWSENSNSYSNITGGGLLAYRVGYNSSMYSVYLRRDGTLPMTGDLNLGGKSIKNIKDMTASGTTTTGALKTTGNASVASDLSVGGTSTLNGPVNINNNLKVKSDTYLNTLSTTGLAKFGSRIATNGLNPNDLPAGWAGGVRTYDLYASGTVGVGTGKTVKAYMNNAGNIYASGNLTAGTIKSNGTIESTGRIKAGEYLHLNGQATLNAKCTPNGLVGRDSTGRVLSCVSGKWQTASGDGLKGIFITITDQTSGYKCVIPNSDTGACACPGSMYDSRYGFLSGTLIAEYDERRCSGSKNEHCYSNFRRLYACK

SEQ ID NO: 22 - protein, 185 aa, Escherichia coli
MVLLNNKRKSKKGFSLLELLLVLGIIAALVVAAFIVYPKVQASQRAQAESNNIATIQAGVKALYTSASSFTGLTNTVAVQAKIFPDNMLSGSGTAAKPINAFKGNVTLAATATGPSSATGSSFTITYDNVPAAECVKIATAAAGNFYITTVGTKVVKAAGGTLDVAATAAACTNATSNTLVFTSI

SEQ ID NO: 23 - protein, 287 aa, Escherichia coli
MYNILFFIFLSIAIPFLLFLAWKQHLKTKEIRSYLLKEGYNIIFNGEGNSYLAFNISNATFRAGNLTSNDYFQASISYIHDYRWEWKEVEAKKINNIFIIYISNIDFPSQKLFYRNNKSLAEIDWAKLQAIFHQPYEIQNDVMQDNNNTHYDFFISHAKEDKDTFVRPLVDELNRLGVIIWYDEQTLEVGDSLRRNIDLGLRKANYGIVILSHNFLNKKWTQYELDSLINRAVYDDNKIILPIWHNINAQEVSKYSHYLADKMALQTSLYSVKEIARELAEIAYRRR

SEQ ID NO: 24 - protein, 176 aa, Escherichia coli
MKKILLVAGAALALAGCGEKGDFEKAINAKIGQDKYCFSLDNNNTSFPIRLAKPRLDSTGTGTNSVILDGFVEQGMMVFEQGYDSNVLGITEEGKKAKVWSTTDGACVGRRAVDGIKEWTEPGNGNQKVVRVTYTWKLVDVPGWIDKKAFASVKGMNEPADGAMNLFKTSNGWKAN

SEQ ID NO: 25 - protein, 157 aa, Escherichia coli
LEAVGIVFLVVLFIIIMTAVDIQKKKHYNSFTEVLDGDILSYECQQTGIVIDTQKHTVRIFNKDKDSTYTFDQIREINYTLSEGGKFYGNGTLRGMNNAAIANWREQLSANKRSGLNILTDDIKNPMWKVNVPLKNLSTSNHELCERWMLVFKKYVF

SEQ ID NO: 26 - protein, 320 aa, Escherichia coli
MEGKLIFCSDAILRFQSDYDETSAVPLLSIQNTIANTDPFFLLRFFHHTVLIEEGTSLASIFLAIELWKALLAAYLDRDVGAYIDEVRKPSGPTTWDIEWIGIDRRSSVYRAYKRQDMEEGEDFSTYFNRERFPTDEFDIESSCDASGFIKGDKERWSISGDVHEIKNLPVILYSKQALMTSAKDGLLKKNVSGVKSSKHGCFVYGDTSFSFSEVMEAIFISGLFFYAPIDAASSLDELKVSLAGLEEELAEVPKVDSNGNETDKEPTIVVAEGAFDSVAAHMETEAEEWQSIKNLCQREGELPIRIGGIKMAEPPEFHF

SEQ ID NO: 27 - protein, 483 aa, Escherichia coli
MRNYMNNNKIITPIIMAGGSGSRLWPLSRILYPKQFLSLIGSHTMLQTTANRLDGLDCTNPYVICNEQYRFIVAEQLRKIDRLTSKNIILEPVGRNTAPAIALAALLMSKSDKSADDLMLVLAADHVIHDEEKFCNAVRSAIPYAADGKLVTFGIIPDKAETGYGYIHRGQYINQEDSDAFIVSSFVEKPNHETATKYLASGEYYWNSGMFLFSANRYIEELKQFRPDILSACEKAIASANFDLDFVRLDESSFSKCPEESIDYAVMEKTKDAIVIPMDAGWSDVGSWSSLWEINDKDSDGNVIVGDIFSHETKNSFIYAESGIVATVGVENLVVVQTKDAVLVSERNKVQDVKKIVEQIKNSGRSEHYVHREVYRPWGKYDSIDTGERYQVKRITVNPGEGLSLQMHHHRAEHWIIVSGTARVTIGSETKILSENESVYIPLGVIHCLENPGKIPLDLIEVRSGSYLEEDDVIRFQDRYGRS

SEQ ID NO: 28 - protein, 381 aa, Escherichia coli
MKKNKKLCLISINSYNELTGGGVYLRTLVSFLQKQNVNLTLIDKKSSGKLFEDNTFQHISFIKGKRQDIISRLFFIPSFYVPYIFSIIKILRKQDILAFHNSRLGLLCLLFRILMPHKKIILFTDNFEYDLIRQKDKNITTFIEKLIVYLNEFIGLKNSDLVSYITRQDKNAMDKFYGIKKSRNLILPVIFSREKPTDVLSAHFINEYNRLNNDNRKKVVFTASFDFFPNIDAANYVLNAAKSNNDYCYILAGRKSTTLNLPDLDNLFFFDNLSNSEMSYLLSACDVFYSPIVLGSGMKTKIAEALSYGLYIYATEHSLIGYDEIINNKECVKKISHLDEEFPKDFKMKSINKQLIMSYQQKYYSHYRFNGHELDIINFDD

SEQ ID NO: 29 - protein, 343 aa, Escherichia coli
MIIYIAAYNGSGGQGGVERVVAQQCNILKNLGVKVIILDKTYFKMSNKIRNKKIQVALYPILVSLYLTLQKLRGVTFKVIAHGYCSPFYRNDILIAHGNMKCYFQTVMNKKPNRLSGSGLLSFYERWAGAFSKNIWAVSNKVKSEWNELYNINSHKIKVVRNFINLAQFDYTDVNEAEYVTFVGRLEKGKGIDDLYYICKNLPDTSFHLVSSIPAPQNFASLNNVLTSIAVPYAKMPEIFKKSRVLILPSYYEGYELVTIEALCCGCPVIGYNVGAIRELYAESFPGVFIANNKEDLAQVAYKLISLDNEKYYHLRQTIYSKRELFSEERYAEILTAAFNEKK

SEQ ID NO: 30 - protein, 253 aa, Escherichia coli
MKKTNLLFRLLILMVFMLGSRAGMGQTFRNVYIYYATFGWNSQVELYLDVAEVSSVYYGTYYQSNYVMTLGNLPNRTWTGTGSSPAPTITVKDGTTVNNSFCPGMEALEAKDSRWECLKLPLDITVDGDVGGCPWLVSIRATSQVVVGSVDKDIYYGPNATVSVCPTVSVTPYDVSWDENYINKTKTLSLQSTGGVIEKTLSTYLMKDGKLCDGSQMNEEGAYCRWVAQMITFSSSGCDNANVTVAPKQASHH

SEQ ID NO: 31 - protein, 351 aa, Escherichia coli
MVYVLIRLVTYGYYRQISEETLVRGARASSYFMESLYGIATVKIQGMVGIRGTHWLNLKIDAINSGIKLTRMDLLFGGINTFVAACDQMAILWLGASLVIDNQMTIGMFVAFGSFRGQFSDRVASLTSFLLQLRIMSLHNERIADIALHEKEEKKPEIEIVADMSPVSLETTDLSYRYDSQSAQVFSGLNLSVAPGESVAITGASGAGKTTLMKVLCGLFEPDSGKVLVNGTDIRQLGINNYHRMIACVMQDDRLFSGSIRENICGFAEETDDEWMTECARASHIHDVIMKMPMGYETLIGELGEGLSGGQKQRIFIARALYRKPGILFMDEATSSLDTESERFVNAAIKK

SEQ ID NO: 32 - protein, 278 aa, Escherichia coli
MAKVNFFDKRILKKFSDYTSTISTIFSLFLIFVDIPTENKLTLGIIFLIILFLLYFGIWFKSNNLSEINLDVEGSIVTVKAGDLFRQDGFKVIAFNEYFDTQVDDVIISHNSLNGLYIDNYLAGSVSDLDHRISNHQFEEDELLEVNHKRKVGKTQKYSLGTIFVNNDYLLTAFSKFDDKNRAFLTMPDYLAFLINFWDKVNRIYAQKSVSVPIFGSGITRIKEHKNISDEDLLKIMLWTFRISEMRFKFPAKLTIVIHKDKIDKINLLDIKSARNGL

SEQ ID NO: 33 - protein, 447 aa, Escherichia coli
MKVKAVPAITFYLSLMLTILVLLFGNEPNKSQYILVIATITVFYIAYITNKITSPASLLVISSFVFLGCRPLLSLFANYDYRIADWFIEGYMDDDVILANYAITLMYYGYTLGLILCKNTEKFYPHGPYPEKQLLKIKFLLTLFFLGSIGMVVKGIFFFNFIESNSYVDIYQSNITTPIGYDFLSYLFYCSFFLICAFHIQFRTNKKFLFIAICIAAFSTLKGSRSEAITFLLTVTCIYFNEVKTRNLRLLITMIFVFSVIFVISEFISMWRTGGSFFQLMQGNNPVINFVYGMGVSYLSIYQSVKLQLLSGGYNVTYLFSQLIITCSSIFNVKLSLPEISYSHLASYTANPELYNLGFGLGGSYLAESFLAFGLIGCFIIPFLLLLNLNVLEKYTKNKPIIYFVYYSVLPPILFTPRETLFYFFPYLVKSIFVAFLVTLYIQYKKD

SEQ ID NO: 34 - protein, 412 aa, Escherichia coli
VHNYLRLKDKIKNVFLGLYILQLFNYIAPLVIIPILIKYIGLGEYGELVYITSIYQIVALIIDFGFTYTGPVVAARHRCETQNLQRYYSIVVLLKSLLFIIALTCVFLLCRLNIVHLSFFGFLSIFLCTIGNILSPNWFLQGIGDFKKLSYSQVIVRITLFIILLVYVCSGGDNVFILSFLQNATLLICCIYLWPNIHISHVVHLKPNECIVEFKKAGNVFIGVIGTIGYNGLIPVLIGNLCGNTSLGVFSIVQKMTTACQSLINPISQYMLSQVSEIKPQDKLFYYRIKKSFFVHLTISIIACLCYMGLGQYVATFIGKVDVSFVIILFASIITIFSSLNNVLGIQFLIPTDNVKILRSINVMAGIIVVSLSWLLISRFDILGGVLLNLIGEFLVFSMLAFIAHRKWGARV

SEQ ID NO: 35 - protein, 320 aa, Escherichia coli
MTSMPASLLHFNNKTHRLMYKKSLRDIVCKKGADMRTHPHLIKQTIDPRLINEINKRTQSNSEKLSNSDKLTYINDNYCEITRNYTSFYGMNMLSIVLFLPIVILCFASVLFFIYDITFNFERYLDTWRYRADSELMLHIAYCAIFFMSFVFAAILNYFIFAPRHYPIRFNRKTGKVYICDYILNDMSHKSTYTFWWHYPFYKHTKPEYKEYDWEQIQGIAFFAQNRHSSYSTIRCIVYESLDSTNIIDTFNLITTESAIDALHMTNHKLWLWINSFMNFDDEFLEKSINTAPGIWGREVNWPEDMNKKSMLSSMDEYIL

SEQ ID NO: 36 - protein, 199 aa, Escherichia coli
MQPLPLKQIHPLWLRCCHWINAVTVAGMICSGWRIYNASPLFSFNYPAALTLGGWLGGALLWHFALMWILLINATIYLTLGICSGRFRSKFFPLSWTSLKKSLTQALYGKLHHTNMQEYNMVQKFAYILIILDGAALLLSGLVLWKPVQFSLLTILFGGYDTARYIHFLCMNIMVLFIIIHLVMVLLVPRALLSMIRGH

SEQ ID NO: 37 - protein, 181 aa, Escherichia coli
MKLKFISMAVFSALTLGVATNASAVTTVNGGTVHFKGEVVDAACAVNTNSANQTVLLGQVRSAKLANDGEKSSPVGFSIELNDCSSATAGHASIIFAGNVIATHNDVLSLQNSAAGSATNVGIQILDHTGTAVQFDGVTASTQFTLTDGTNKIPFQAVYYATGKSTPGIANADATFKVQYQ

SEQ ID NO: 38 - protein, 476 aa, Escherichia coli
MGTGYFLVRGDKTTCGGKIIEGADDHTIMGIPQARDMDRVTCGRYPGMFIIVGGVPETDIHGRLMAGTLDSQSSCPCKARFIASMMDDTYETDDGGNEPEQHAQSAKKDLTSGSDSSSDDVKLDYRIKLSGNKILTPLNIPDYKEMISGGSTKNTEKIDFTITNKGDETEALSLEVLDGNEVIYSERQTGKYCDKGEHAWQWDGYSNQGILDTTKIKSKSLLVRLIALCGDMMIKVDYPLKTSANEEDWVDVKVDKNQKIVDVSWRVEFEDDGVSHKDDRIAPVGFEKLKQLAKDGMEYYWARNGMRNPGIGNNITTPHGKYNVNISVSINIDPAMDSFDLIEEQDLESVRSSAFMGRMNIYFNRGYYEYGRGYKKGADPVFKAGLEFKLDVAHETGHMILKSYGGNTYSWEHKGTSNLVPPQYALPHSVYPGTGEIDLMKYYDGHIYTSDLYARSVAVENDVCAMIWLSRVKFHD

SEQ ID NO: 39 - protein, 182 aa, Escherichia coli
MKHFIATAVMASSLLFGSNALAASSSAAQKESSPEQSGGTFKYSTDSKIATEQKNVYDTIIKYQNALNAGDTKTILGLFADESYSQWNDKLTADNTEKRQGQYDDLFKREKFETDFAFDSIWINGDTAVVRTHHHVGSVVTNFKEQKTIIDLNREVFVLSKINGEWKIFLYTFNTNPLQGVA

SEQ ID NO: 40 - protein, 102 aa, Escherichia coli
VLFSDRHITVSIMTMPSGGGIPVIPCHDPVTSPGCLKPERPGPTTLPVTQSRCSTPCIRLFCAEGYRKDNNVTARMHQIKQCILSVSSHWSGLWGEIQVLIL

SEQ ID NO: 41 - protein, 246 aa, Escherichia coli
MNKVFVVSVVAAACVFAANAGAKEGKSGFYLTGKAGASVVSLSDQRFLSGDEEETSKYKGGDDHDTVFSGGIAAGYDFYPQFSIPVRTELEFYARGKADSKYNVDKDSWSGGYWRDDLKNEVSVNTLMLNAYYDFRNDSAFTPWVSAGIGYARIHQKTTGISTWDYEYGSSGRESLSRSGSADNFAWSLGAGVRYDVTPDIALDLSYRYLDAGDSSVSYKDEWGDKYKSEVDVKSHDIMLGMTYNF

SEQ ID NO: 42 - protein, 335 aa, Escherichia coli
MKKWFPAFLFLSLSGCNDALAANQSTIFYSFNDNIYHPQLSVKVTDIVQFIVDINSASSTATLSYVACNGFTWTHGLYWSEYFAWLVVPKHVSYNGYNIYLELQSKGGFSLDAEDNDNYYLTKGFAWDEVNSSGRVCFDIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTTPAYSHGQQFSVGLGHGWDSIVSINGVDTGETTMRWYRAGTQNLTIGSRLYGESSKIQPGVLSGSATLLMILP

SEQ ID NO: 43 - protein, 158 aa, Escherichia coli
VLMSQHAHAADNLTFKGKLIIPACTVQNAEVDWGDIEIQNLVQNGGNQKDFTVDMNCPYSLGTMKVTITSNGQTGNSILVPNTSTASGDGLLIYLYNSNNSGIGNAVTLGSQFTPGKITGTAPARKITLYAKLGYKGNMQSLQAGTFSATATLVASYS

SEQ ID NO: 44 - protein, 182 aa, Escherichia coli
VQITEALISEPGDIRRFIQHAVDHWPRLLAVHFILHSTEGNIYGQQIHAFCTSFYRQLHERITESNHTASPSSSVVLRWLREQHGGATIRCLLLLSQTSICHPRASVTVDEQCSQVVDLLQHSWQVISAGGQCRVERCFRVARGDTSGQYVALKTVALSLGLPVVTAITHRPVQRCTLITAQ

SEQ ID NO: 45 - protein, 145 aa, Escherichia coli
MLLSGLDLSRWCEYHLMQWSLSRQMTSNRARYLNQDLIVLVRCRKPVIFRVGICVGIGWCCLLLTILNHEVKSHVRVRPSTTPYANRVAQERLFLCLKSPISVQYIGLPEKQFSILHFVVDGPKFALAGSCIVHITWAGWFRYAT

SEQ ID NO: 46 - protein, 109 aa, Escherichia coli
VPGSNNNNLPRQISKKNSDTLILESCLFYYVCRKKLRKRRTINTQLSHTDWQRKCLQEQVICHGKANQKYRLILVFKPFLLVSMIQRLSKKLGNSTTLGNYGLCPAALI

SEQ ID NO: 47 - protein, 1624 aa, Escherichia coli
MNKIFKVIWNPATGSYTVASETAKSRGKKSGRSKLLISALVVGGLLSSGVQASTALDGGGASESTPLTDTWIAIGQGAKASSVDTSPSGGPGTSSIAIGQEAQSGIGSTSLGFKSQATGERSVALGQTADATGNRAIAIGSGAKSTADYSLTLGNGSKAFAENATAIGKDARAEGKGSQAFGKVALATGENAIALGSNSNASGDNAVALGSTSQAYGDSSLAIGELSETNAENAIALGKLSNASKINSIALGSNSTASGEGSVALGENSFAGGINSLALGSQSNANGDNAVALGVGSVAEQDNTVSVGNSTTQRKITNMAAGLIQPGSTEAINGSQLHALSASVAARLGAGAGVNGDGSVNAPSYKLKSGIYNNVGEALLGIDNDTLHWDKTEGAFSASYIVKNADGSPKSSIAQNKIINVAKGTISATSTDVVNGSQLYDLQQDALLWNGTAFSAAHGSDTTSKITNVAKGTVSATSTDVVNGSQLYDLQQDALLWNGTAFSAAHGTEATSKITNVEDGTISDTSKDAVNGSQLKETKDDVATNTANIADNTADIATNTANIADNTTDIATNTANIATNTTDIATNTANIADNTADIATNTTDIATNTANIADNTANIATNTTDIATNTADIATNASSITTLNADVDTLEKDALLWNGTAFSAKHGTEATSKITNVTAGDLTAGSTDAVNGSQLKTTNDNVSTNTTNITNLTDSVGDLKDDSLLWNKTAGAFSAAHGTEATSKITNLLAGKVSSDSTDAINGSQLYGVADSFTSYLGGGADISDAGVLTGPTYTIGGTDYTNVGDALAAINTSFSSSLGDALLWDATAGKFSAKHGINNDPSVITDVANGAVSSTSSDAINGSQLYGVSDYIADALGGNAVVNADGSITTPTYAIAGGSYDNVGDALEAIDTTLDDALLWDETANGGNGAFSAAHGKDKTASVITNVANGAVSATSSDAINGSQLYSTNKYIADALGGDAEVNADGTITAPTYTIANTDYNNVGEALDALDNNALLWDEDAGAYNASHDGNASKITNVAAGDLSTTSTDAVNGSQLNATNILVTQNSQMINQLAGNTSETYIEDNGASINYVRTNDNGLAFNDASASGIGATAVGYNAVASGESSVAIGQGSSSTVDTGIALGSSSVSSRVITKGSRDTSVSEEGVVIGYDTTDGELLGALSIGDDGKYRQIINVADGSEAHDAVTVRQLQNAIGAVATTPTKYYHANSTAEDSLAVGEDSLAMGAKTIVNGNASIGIGLNTLVLADAINGIAIGSNARANHADSIAMGNGSQTTRGAQTNYTAYNMDAPQNSVGEFSVGSEDGQRQITNVAAGSADTDAVNVSQLKVTDERVAQNTQNISNLNNQVTNLDTRVTNIENGIGDIVTTGSTKYFKTNTDGADANAQGKDSVAIGSGSIAAADNSVALGTGSVADEENTISVGSSTNQRRITNVAAGVNATDAVNVSQLKSSEAGGVRYDTKADGSIDYSNITLGGGNSGTTRISNVSAGVNNNDAVNYAQLKQSVQETKQYTDQRMVEMDNKLSKTESKLSGGIASAMAMTGLPQAYTPGASMASIGGGTYNGESAVALGVSMVSANGRWVYKLQGSTNSQGEYSAALGAGIQW

SEQ ID NO: 48 - protein, 306 aa, Escherichia coli
MYEVCVTFYMRVKQVITTFSKGYSQVLLQNNVVSGLFFFLATGIASFNMGHPEILYFSAISAALSPFFAWCLRYPDEEINEGIWGYNAVLYGIACGMVVPVSVSGITVLIVGILGIVLFTPLLSSVLRGLPVLTAPFIIATWLLHSGIMPHDMSVPSGEGAGSVLLKNYPVAFSGNFMEIFMFPDAVGGILVMLGLLAGSRKLLMITVVASFLSVVVSFCIPELSPDKISAGLYGYNAILTAVAVYAFAGDNQFDNIFLGCGAIILAFIIPVLIGPLIPVPLLTSPFVISTWLYILAQRRCMSSHI

SEQ ID NO: 49 - protein, 133 aa, Escherichia coli
MSSKTKCWLWMLLVILSETSATSTLKMFDNSEGMTKTLLLALIVVLYCICYYSLSRAVKDIPVGLAYATWSGTGILMVSTLGILFYGQHPDTAAIIGMVIIASGIIIMNLFSKMGSEEAEETPVTNLDKKIAN

SEQ ID NO: 50 - protein, 386 aa, Escherichia coli
MSIKLRGGTWHCDFVAPDGSRVRRSLETSDKRQAQELHDRLKAEAWRVKNLGESPKKLFKEACIRWLREKSDKKSIDDDKSIISFWMLHFRETILSDITTEKIMEAVDGMENRRHRLNWEMSRDRCLRLGKPVPEYKPKLASKGTKTRHLAILRAILNMAVEWGWLDRTPKISTPRVKNGRIRWLTEEESKRLFAEIAPHFFPVVMFAITTGLRRSNVTDLEWSQVDLDKKMAWMHPDETKAGNAIGVPLNETACQILRKQQGLHKRWVFVHTKPAYRSDGTKTAAVRKMRTDSNKAWKGALKRAGISNFRFHDLRHTWASWLVQSGVSLLALKEMGGWETLEMVQRYAHLSAGHLTEHASKIDAIISRNGTNTAQEENVVYLNVR

SEQ ID NO: 51 - protein, 583 aa, Escherichia coli
MYFPLIHYGIIYCDTLLSHPQKHLPKRKLTAGQSFAGLAWKQYHVTDTAQHRLHLRRFVNMNQPHADNLQGQHPDRAICSADEDQYGFIHIATQLAVAVKGIGREGSAVIGIEGPWGSGKTSLLNLLRNALVEQIEECTFVLTISPWLDGSNTSLVASLLLPVANIIAAEEEQRLAPEERAALRRRKSLTRTAKTMIDYTRATARNLATVASAAAVIPGVPDASGALKALSETRWLKEKEKTTAEMRTEIAKKIDELDLSFIVLLDDLDRLEPAQAVEVIRLVKSVGDFPRFRYLLCYDKAILSQAISLGLGVPDGNLYLQKIVQISFCLPRPETFVLRRKFRDAAAALYRKVNGHAPDRIVLEQLTQVADVYGAALKTPREVQMVLNALTFLYPGMRDYVYFPDLCFLQLLRTTNAGLYDWVEEYLSERAVVAAGDGHVSELEQAEMAESLKSHLARYFPAEAHAVHALARWVPGITGWKTDSPIKLFVPTPEQDSALLTAGKRLGSQAYWLYYFAFSAPQNVLSPEMLNEIFDIAGYPEQQHRLAERLLGYIQSKGLSSRTWFEHILTQLTTQQIATVNDG

SEQ ID NO: 52 - protein, 240 aa, Escherichia coli
LRLYMTVFSFIKEVLSPHIRLLYSPQELGFNDAGLIVLNHFGYDNNIYYSVTLTRKEITSFQNTSENTHYFAYSKTIKKTIDRIFKITGEKITIGNITPSINGKVSVTLILSLLEMKLIDFIADDNTDVRVRLTVLNMLNNNTTTIRELAASVYLQEDYLKRIFRRYTGTGLYQFYNWIKLLKAISKIQNNDKIKKIAIELGYSSSSSFNYSFKKEFSIGPRDYIKEYLHNKISCMAVHE

SEQ ID NO: 53 - protein, 197 aa, Escherichia coli
LNILAPIFHDCSSLSLGLLCARSGLLTGFDVRYWAIRIAATKMRARLMNYFIRPATPDDINKIIEFTTQEAQESEGSVIDLVAVERGVSSAFSENPRSKYWVMEDSSGTVIANTSVVKEWSDFHGNDYWWIKSFYIAPEHRGMGLADELIKHLIKEAKSEKALELRLYVHGDNGRAIRAYERCGFIESPYTIMKVGL

SEQ ID NO: 54 - protein, 340 aa, Escherichia coli
MIRYSGIFLSLLMLSGCATEALKNEQAETVITAGNKAVAANASFFKQYYASRENFLVNFYATNPDCALRPDYETIMENNTNKESLCLQSEKGGKTSYKDITKIQLATTEKQKLLASLEVVNSMSSYVELLTVGMDVEDSKQIILLNQALDHANNAQKLLGFSEGEELTKSAVAVKDLVQYFNGLYKEKIKSENVGKAVGSDWPRLQRNLQDLLDDIATKQQQMDRNNFANISNNFYYYNKARANTKNIEFKTLKSRQEFLSAVINLNESRLRSSDPAAPAIQAINAFMDKGNHLQDLYSNKSLSKADLERRNEILRAQVISGLQASEELAKLLIASGVML

SEQ ID NO: 55 - protein, 102 aa, Escherichia coli
MNDVQNRRILALLNMLYYALGNRTTCDICHKYFILVLSVMNNLTINYGSQTSPELTGISYEDANDLITELIMFIEDVNADNDMEYPAHLNKIQVLLNFAVAN

SEQ ID NO: 56 - protein, 2458 aa, Escherichia coli
MQRKTLLSACIALALSGQGWAADITEIETTTGEKKNTNVTCPADPGKLSPEELKRLPSECSSVVEQNLMPWLVTGAATALITTLAIVELNDDDDHHRNNSPLPPTPPDDDSDDTPVPPTPGGDEIIPDDGPDDTPAPPKPIAFNNDVTLDKTAKTLTIRDSVFSYTENADGTISLQDSNGRKATINLWQIDETNNTVALEGMSADGATKWQYNHNGELVITGDNTTVNNTGKTIVDGKGATGTEIAGNNAVVNQDGELDVSGGGHGIDITGDSATVDNKGGMTVTDPDSIGIQIDGDKAVVNNDGDNAISNGGTGTQVNGDEATVNNNGSTTVDGKDSTGTEINGDKAIVNNDGDSTILDGGTETRITGDDATANNSGNTTVDGQGSTGTEIAGNNAVVNQDGELDVSGGGHGIDITGDSATVDNKGGMTVIDPDSIGIQIDGDKAVVNNDGDNAISNGGTGTQINGDEATVNNNGSTTVDGKDSTGTEINGDKAIVNNDGDSTILDGGTGTRITGDDATANNSGNTTVDGQGSTGTEIAGNNAVVNQDGELDVSGGGHGIDITGDSATVDNKGGMTVTDPDSIGIQIDGDKAVVNNDGDNAISNGGTGTQVNGDEATVNNNGNTTVDGKDSTGTEINGDKAIVNNDGDSTILDGGTGTRITGDDATANNSGNTTVDGQGSTGTEIAGNNAVVNQDGELDVSGGGHGIDITGDSATVDNKGGMTVADADSIGIQIDGDKAVVNNDGDNAISNGGTGTQVNGDEAIVNNNGNTTVDGKDSTGTEINGDKAIVNNDGDSTILDGGTGTRITGDDATANNSGNTTVDGQGSTGTEIAGNNAVVNQDGELDVSGGGHGIDITGDSATVDNKGGMTVADADSIGIQIDGDKAVVNNDGDSTILDGGTGTRITGDDATANNSGNTTVDGQGSTGTEIAGNNAVVNQDGKLDVSGGGHGIDITGDSATVDNKGGMTVADADSIGIQIDGDKAVVNNDGDNAISNGGTGTQVNGDEATVNNNGNTTVDGKDSTGTEIAGNNATVTQEGELTVSSGGRGIDITGNNAKVDTKGKMTITGTDSVGVSINGDSATLTNTGDIDVSNSATGFSLVTNEGIISLAGSMKVGDFSTGMALSGDNNSVTLAAKDINVTGQKATGVNISGDSNTVDITGNILVDKDQTADNAVDYFYDPSVGVNISGNSNTVSLDGKLTVIADSELTSRKYMEFDGSQENISGLTVSGDGNTVNLNGGIQFVGEKNALADGSTIADKRSYFGKTPLVSVDGQSKVYLNGDSTISGSLPLGYANILQLSNKAALEIGSDATFSMQDISVYEHYFTQTPQIIKVDTGSQVVNNGDVDIWNISFAGIWGENSTGINNGNITLSQYDYSSPETSFSEPDHMAFLSSSGGSAVNNGTITAKVMEQHSVLNMGSAAGVADPRVFNNSVSSMMGMEAYGKGTVLNSESGVIDMHGRGNIGMLAVDDSAADNAGKITLDTLWVDQNDTTTLRTDLPSSTAIDYGVGMATGTNSGGGARSNGVATNQQGGVITVYNAGAAMAAYGASNMVINQGIINLEKNGNYDGGLGANMLVGMAVYNRGTAINDKTGVININVDTGQAFYNDGTGTILNYGEINLLGSPMDSADSHMGAIPENLDLLTALTGSGETDMRTASSGGFVTTKALANYGNETLNSNVAAKAWLYNQDKANLTINGELSIGQGLENSGLLDSDTISAAANVYNRASGSIITDQLSLTGSNSFFNEGNFSGSVAGSSYKQNVVNTGTMAVMADGKSLISGSFLLYNEAGATLSNSSSAVSGGENAIVNVTRTGDSLAQVNRGTITAINGYSAIKTASTGSNSNGKWIWNTDTGVISGVNPNAPLIDLGRGYNFANAGTINVQGDGAVAISGGTTSYTVQLVNSGTINVGAAQGKADGTNGTGLIGIKGNGSDTTINNAQSGVINVYADNSWAFGGKTKAIINNGEINLLCDTGCDIYAPGTTGTLNDHNSTTDIIVPAATSTPTQGSVPTVPADSSAQQKLTNYTIGTNSDGTSGMLKANNLVISDNVKVNTGFSAGTADTTVVINDVFKGENISGAENISSSTVMWNAQGSTDASGNVDVTMTKNAYTDVVTDSSVNNVAQVLDSGYTNNDLYTSLNVGTTAELNSALKQISGSQATTVFNEARVLSNRFSMLSDAAPEVANGLAFNVVAKGDPRAELGNDTQYDMMALRKSLTLTEHQNLSLEYGIARLEGNGSDTAGDNGVTGGYSQFFGLKHQMAFDNGMSWNNALRYDVHNLDSSRSIAYGDVNKTADANVKQQYLEFRSEGAKTFELREGLNVTPYAGVKLRHTLENGYQERNAGDFNLSMNSGSETAVDSIVGLKLDYAGKEGWSANATLEGGPNLSYVKSQRTASISGAGSQRFNIDDGQSGGGFNSLATMGVKYSSQESALQLDAFHWKEDGISDKGVMLNFKKTF

SEQ ID NO: 57 - protein, 505 aa, Escherichia coli
MYKKTTLAVLIALLTGATTVHAQTDISSIESRLAALEQRLKNAESRAQAAEARAKTAELQVQKLAETQQQNQLTTQEVAQRTVQLEQKSAENSGFEFHGYARSGLLMNDAASSSKSGPYLTPAGETGGAVGRLGNEADTYVELNVEHKQTLDNGATTRFKAMLADGQRDYNDWTGGSSNLNIRQAFAELGALPSFTGAFKDSTVWAGKRFDRDNFDIHWLDSDVVFLAGTGGGIYDVKWNDTFRSNFSLYGRNFGDLDDIDNNVQNYILTMNHYAGPFQLMVSGLRAKDNDDRKDANGDLIQTDAANTGVHALVGLHNDTFYGLREGTAKTALLYGHGLGAEVKGIGSDGALLSEANTWRFASYGTTPLGSGWYVAPAILAQSSKDRYVKGDSYEWVTFNTRLIKEVTQNFALAFEGSYQYMDLKPKGYQNHNAVNGSFYKLTFAPTLKANDINNFFSRPELRLFATWMDWSSKLDDFASNDAFGSSGFNTGGEWNFGVQMETWF

SEQ ID NO: 58 - protein, 272 aa, Escherichia coli
MPARGFFFIVIDEPGKDGFLVRKIYASPNSPYLTKMNLDPLRLLSETEPERYGLMPKDYSAPTTVAEHVMGNNKSSLISTSSIFPDGSPRFEGKTIFIDVHKAKNAGLKLISTAEIITALEEYKKQYPHLAKRVEKISGYVDDIDKEVLIEGRKVPAQTIFTPESLKTTQNIVKAGRVVQVFSIFFTAYDLEQATEKSFETKSIKPIGAEAIRQIGGWGGAVAGAKIGTVIGASLGVTTGPGAVATGIIGGIVFGAAGYFGADWVADYIDEN

SEQ ID NO: 59 - protein, 306 aa, Escherichia coli
MFLTNTSRFLFLFLFLMHKNKLYAYDNFSFNADVHYGYGKLLWHDKKPVIYPLASLSDYTYYNEFDPFETIYYVGQYYKISLNESKRIQEQKDIYAHLQLKHNKSLITANVTLHNKSNSSYYVHRMNLPASIHESPYSPLCSGSFLIVTNNIRLDYIRKITCRFDLWMKKSDWIEISPGEKLSYTVNLNDYYAFLPAKHQYDIGTVEFTLVNSNWFLEQHIYDLFFAIVDERHYKQCKNTSGIVRKYIYKSWQLCETYRGRWSPLQELMNRFHYEGGSENIFQIRTNQENININGDKIKSLYSSMQ

SEQ ID NO: 60 - protein, 330 aa, Escherichia coli
MKYVKKILGISFFSLLPLSAEAESYCNLLWASNTLPSASVNVSFDGNTSGIFPLNLQAGGLSTVIKRAAKNMDTFVTLDGTYRYWIQYPEAWQTTPDGLKYRITSELEVSGTQTAGVKTVVTSVGYHTWVNTYGCRDVGGTYDFGVASVSGVNIEIDRGTAWPGVYSIQLPVKVAYEENKGNYDGKNGGGWREFPVSMKSFSPVDSKGISITISSKCNVGEQSLSVNMGDNITPDEAKSGVEKKVNFSLTCNAPAKVSLSLKGTDIVDGVNNKTKCGSGSCSLNFDNDSSSKILEVNQGTYQVPITVRFQDANPVAGGFDGSAVLSVDIL

SEQ ID NO: 61 - protein, 172 aa, Escherichia coli
LRITVFLLTFLSFLSDLWAVDIPINITGTIIIPPCQINNSNPVDVDFGNIRVSELDTKEHIKVVSFPVYCPYHQGEAYVKMTGQSMTGKDNVLATNIDGLGIELYQGGEGTGNHLILGSGSSGYGYEVINALSEKNVERTTFTFTAKIYKAEGVTINSGEFSASALINIVYL

SEQ ID NO: 62 - protein, 187 aa, Escherichia coli
MNRATIGFYLAVVLGSCSMNGVSQADELLTRDDFFVADESRHQWVTEHNGRTGALNVKGALVSSPCILDTPEVNFPLQKDKGRYVLNLKVSRCGDGHSDIPERQSTEHGNIVVKQSAVLKTGKDQILLSAWRNGGLSRKNLQHGDNQITYYINEAQYQKIAKTQMPVTAHKTSDSNRGVMHLNIMYE

SEQ ID NO: 63 - protein, 290 aa, Escherichia coli
VCGGHPGTSGPGTTVAAALSSGEVTLYTPAIVCISRQKNVKKQRAENMQKMKPALKKTLMAVACLSAVPAAQAAVSLDRTRAIFNGDEKSMTLNIANDNKQLPYLAQAWVENEKKEKITTGPIIATPPVQRLEPGSKSMVRLTSTPDISRLPQDRESLFYFSLREIPPKSDKANVLQIALQTKIKLFYRPESIKAKPNAVWQDQLVLNKTSGGYRIDNPTPYYVTVIGIGGSEKQAREGEFDAVMLAPKSTQMVKSGTYNTPYLSYINDYGGRPVLQFSCGGSRCTAVKK

SEQ ID NO: 64 - protein, 856 aa, Escherichia coli
MLKRVGCALQSVFVLNRLHIMKKNKSTFTINFITYSLMLSLAGVPVYAVDFNTDVLDAADRQNIDFSRFSRAGYIMPGQYQMEIRVNGQDISPSAFQIAFLEPPFSDSDNEKPLPEPCLTPEIVSRMGLTEASQEKVTYWNNGQCADFRQLSGVEIRPNPAEGMLYINMPQAWLEYSDASWLPPSRWDNGIPGLLFDYNINGTVNKPHQGKQSQSLNYNGTAGANFGAWRLRADYQGNLNHTTGSAQGTDSQFTWSRFYMYRAIPRWRANLTLGENYINSEIFSSWRYTGASLESDDRMLPPKLRGYAPQVSGIADTNARVVISQQGRILYDSTVPAGPFTIQDLDSSVRGRLDVEVIEQDGRKKTFQVDTAYVPYLTRPGQVRYKLVSGRSRTYEHTMEGPVFAAGEASWGISNTWSLYGGSIVAGDYNALAVGLGRDLSKFGTVSADVTQSVARIPGYDTKQGKSWRLSYSKRFDEVNTDITFAGYRFSERNYMTMDQYLNARYRNDFTGREKELYTVTLNKNFEDWKASVNLQYSHQTYWDRRTSDYYTLSVNRYFDAFSFKNIALGISASRSKYLNRDNDSAFVRLSVPWGTGTASYSGSMSNDRYTNTVGYSDTLNNGLSSYSLNAGVNSGGGQPSQRQMSAYYNHNGSLTNLSASFSAVENGYSSFGMSASGGATVTMKGAALHAGGMNGGTRLLVDTDGVGGVPVDGGRVYTNRWGIGVVTDVSSYYRNTTSVDLNKLPEDMEATRSVVESVLTEGAIGYREFEVLKGSRLFAVLRMSDNSYPPFGASVTNAKGRELGMVADSGLAWLSGVNPGETLNVGWDGRTQCVVDIPAHPDPAQQLLLPCRQVK

SEQ ID NO: 65 - protein, 195 aa, Escherichia coli
MLRRMILSVLFLWCAVAQALPSASFPPPGMTLPEYWGEEHVWWDGRASFKGQVIAPACTLSMEDAWQEIDMGTTPLRDLQNSPAGPEKKFRLRLRNCELTGAGKQVYTATRVRVTFDGIPGETPDKFSLTGQAEGINLQIMDNYGYPARAGKSMPPLILRGNEDGLDYSLRIVRNGYPLKAGDYYAALRFKLDYE

SEQ ID NO: 66 - protein, 177 aa, Escherichia coli
MLKSTLGIAIAFSLCAVAHAANQGQGVVNFKGTVIDAPCGIAPESADQTIDFGQISKSHLKNDGISVKKDLDIKLVNCDFTDPTAKKTVSVTFSGTPVNGHTDELGTAGDTGTAVVVSASDGSMVKFDGTTSSNATQLQDGDNTLKYTTWVKKSSAAGTEVKEGDFTAVANFNLSYQ

SEQ ID NO: 67 - protein, 128 aa, Escherichia coli
MCNATLIAISGYGFIPSVLKIFFTGETGKWKDFRDDVRIRYSDGIMLTLITAQNNERIMEVKTKEDWLYQFRRCSSRETLEKVISHTRYKLTPAEMEAFNSAVDHRLAELTMNKLYDRVPASVWKYVT

SEQ ID NO: 68 - protein, 224 aa, Escherichia coli
MRAYKFRSASQIAFTLDIIFNRRLFCSDWKMLNDPMEGMFVFSHQTSDGYDYKKEIQQIIEEKKRLKVCSLSATYESHLLWAHYAGGVDGCAIEIEIPDNHHSIKQIEYRGVFANLAMPNIYSPDELANQVLSSKYREWEYEQEVRILQTNEWFYLDKPVRRVIAGHRMPSALFEAMNIICNSMGIEFCRTGIGDEGIDADYVEPSKILTPNNHLDWDVLKNDR

SEQ ID NO: 69 - protein, 360 aa, Escherichia coli
MRNRLIAVILCLFGMVAGGHATPNVTAEITYDLAAGRADYYFWNEEESPGNNAYMWYECSYPNIQQTCSDSGNFSTVQIYLTEQRSGMRWPITLKGFKKAYVDDGIPGCEGYVAQTDLRDSNRYKCYVSGDSTPHYADKKFLTLYLEQAEMKKLPIGGVWKGQVKLHSNSPSVDYFADITLNTLDPNHIDVFFPEFAHATPRVQLDLHPTGSVNGNNYAQDLTMLDMCLYDGFNGNGLSYEILLKDEGKTAAGRSNGAFSIYRQGASSTDEGERIDYRVKMYDPESGGQIDVRNNESMVWTNINLKHVRPVVLPGIRYAVMCVPTPLTLVVDKFNVTAKQAGYYMGKLSVIFTPSLPTIN

SEQ ID NO: 70 - protein, 119 aa, Escherichia coli
MDYRTAMDKKLLALLILASLSPAKATLTKIPAGFEVIAQGQQEYIEVYFAGKSLGKYYAMVNLDTVTFLDSSSLYSKLELSTDDQKIAHTVKEKLSQPLARHGELACGFVRTDSGCGYS

SEQ ID NO: 71 - protein, 166 aa, Escherichia coli
MKKVFAKSLLVAAMFSVAGSALAVQKDITVTANVDAALDMTQTDNTALPKAVEMQYLPGQGLQSYQLMTKIWSNDTTKDVKMQLVSPAQLVQSVDASKIVPLTVTWGGEVIAADAATTFTATKIFASDALTNGSLAKPLMFSQATKGVLETGIYRGVVSIYLSQAL

SEQ ID NO: 72 - protein, 238 aa, Escherichia coli
MVAKINRLLRLGPVLLLCTSFFAQSNMTVYPMSVSINSQGEGSVRVISKTNEVQYIKATVFRIDNPSTPQEKEIEVTSGDANQLVIMPPKFALPGGSSKTVRFVAMEPEQKEKNYRVKFEAVPSIDDATDNKKDLSMQLTVNLIWGIVVSVPPQQPVAKLDINADKKLINNGNQRLKILTIAYCKNSGSAECRTQTVNKNIFPGQERVLESISGYDKIVVKYNNWITKDNGEFELAVH

SEQ ID NO: 73 - protein, 183 aa, Escherichia coli
MKIKVIALATFVSAVFVGSAMAYDGTITFTGKVVAQTCNVTTGNDGNFTVTLPTVSNKALASPGETTGLTPFSINLTGCTSTVDGAGHVKAFFEPGSSTDFVTNNLKNIASDNAAGDVQIQLLNSDGTSTIFLGKDFAGQDVHSETIGGDGNATLRYMAQYYATGASTPGNVKTTVNYTIAYE

SEQ ID NO: 74 - protein, 560 aa, Escherichia coli
VSKPAFISSGAVPDSCFMLCMDCVSSLQQNKGVRAMYPSTGHYSQPNLPALTSTIQAIKRISPYPTDTLPPVLGNVIRSLHNSTGIPTELIGNVVLATVSLTCQSLVDVIQPHTDMPEPCSLYLMTVAESGEGKTTINKLVMKPCYEFAASLIQQYQTQNNDYKNQLNIWKIRQRVLEGNFRQAIKKRCSGKDEREEIHQHFLSRPQRPPYPNFIYEDTSLKALVEGLSDYPWAGMISDEAIIFFKSHLKNNPGLLSKAWDGEIIDFRRADGECYHFIPHLTFSLMPQPGVFSEYIRKNTVSARESGFLSRFLFVCPDRNSAECLNTGTGAYTDYNLNVFHKRINELLDRHNNLNSGTEIRKKTLRLSDEAIADWQENHAVIQRMISPGGEWEHIRDIALKASANILRLSALFNYVHDDREDAIHSHSLKAVIYLVNWYLGQAEKLFYPMSERYQFENDVRRLYAYIFDKVNQNNGYPFPKNHLEKYGPHSLRRAEKLTPVLNQLIKQRLVCVIQIYPGRALYVALCNQNGIIIAPYNVHYTVIQSQENTWGRPYDVTIL

SEQ ID NO: 75 - protein, 217 aa, Escherichia coli
LLQDKRGGILDILFLASRNSQMKKMIMPLTMVSVLMSGSALAAIGDNGQANNNDASQAELHFTGKLTSSLCQVATSDVKKEIYLGELSQAALTQGRGRGPSQSFSVSLVNCDPQVTTIKYSLQDKNGSKGDYLVNQSGDTMAKGVGVYIEDNLNQPLQVDGAENTVTVQNNGTGALPNQVIPLAAYIGSTSGKVDDVGTVTMGLVDASAVMTIRAAQ

SEQ ID NO: 76 - protein, 233 aa, Escherichia coli
MRYQYSVLFVFLFSGVVQAAEFFGPRESRLLFEEKKGNTFYRIDNSDKKLPWLVQAWIEDASEKKTTALTATPMVFRVEPSSVFTVRVVKTGALPEDRETLFWAVSNSLPGGASTKQDNEEGKISAKISLAYRFKVPLIYRPAALDNFRQEPEKLEWTYNGKEGLKLYNPTRYVVQLHNVTANGREFKGKGVSFILLPMSGKNVSAAVNKGTRIKYGVINDYGAVKEYDGVVK

SEQ ID NO: 77 - protein, 143 aa, Escherichia coli
MMVLLSNALKKAVLSLLASCSIVVFASESELGVAVEPMRLSIKPGEMTYFTVINETEREYIVTTRVVSDSGIDDKDKVFVFSPPLKHLKKREQSVMGVVYLKNGRENKMKYYLSVSFVPKLSEDKVKISIPVVLVHQIPLMFE

SEQ ID NO: 78 - protein, 835 aa, Escherichia coli
MNKYYLLIILFFVRSTKVFAEDYFDPSLLATDIIGEGNIDLSAFSRPGGGMEGEQEVAIYVNDEFYSRNTLFFKNTLDKGLLPEFTPGFFDELLSGDFLVSEEDKTISSSDFLKKVPYSDINFNQGMSRVNVSIPQAYLGDGAKLISSPDTWEYGGPAFLLDYNISGNRNDSGNYDSRSLYISSQMGVNLMKWRLRTSSSYSNYKTNSVWGGARSEQNSFYNTYAERDISSLRAILRLGEVSTAGLILDSVPFRGMKLSSSDDMLGMRLRNYTPTVRGMASSQAVVTITQNGRQVYQTNVPAGPFELNDFYLSGYSGDMLVTVREADGSEHSFLQPYSTLPEMKREGVSGFEVSVGRYDNNGAEHYYDAESFVYGNWSRGFARGVTFFAETLQAEKYQSLGGGSTLSLGRLGAASADISLSRADKYGDIRIGQSYGFKYSKSQIETGTTVTLATYRYSTENFYTFRDFVSKTDTARYIWENKLKSRMTFSLSQSLGEYGYLSANASQQDYWNSREVSRNYSLTHSFSWNDIYFSTTLSMDDQRGRETGHLSNKQAGIYASVPLSKLLPRTDPTSSSLTWSTSHADHKVRNSVTLDGKVPESDVRYRVGGSWGNGTTEGSRMASVSWTGDHASTSLGYTRVGKYRTLDYSMSGAAVMYPWGIAVGNSSVTGDGAIVVETPGAKGVRTSTGYKTSWLGTALISSPQKYTENRINLYPDGLPSDTVLGETSKTAVPAKGAVVVLDYTVFRGSQVVFTLRQTDGNPLPFGTVITLDGVSRGKENSGIVGEEGRVYMAGIPEKGTLTASWGLNKTCSIPFRINQHKAEAVIREVQGVCRV

SEQ ID NO: 79 - protein, 150 aa, Escherichia coli
MPCMMLQIFITILLLLPLSVTKADILYPWPSDTEVRLKISDEKGQSRGDLRVTNPGDALWLVQAWAEDEKYRRYSVIYPSVYRLEPFSAYALNIYPGNNIMPERLKWFLISFIPSIVEKNKNQLIIPVTYRLKIIDDVVCKGIEKVAGEC

SEQ ID NO: 80 - protein, 182 aa, Escherichia coli
VSVKSMLKKIPCIILMSLPGLISAAEITKQIELTLKVNVLKPVCKLSSGQQTINFGDFDVLDVITKSSKVNGSATFRFTECSAVNNIKIKFKQAGQSPVPDIENNYIPNAKGDIMAKGVAVKLLDDQKKEVELDKIMSVSVGENQISKDLTLNAQVISVNKTGEGISPGMLQTAIGMEISYE

SEQ ID NO: 81 - protein, 421 aa, Escherichia coli
MLKCLCFGYFTKSLMQRIIKCTYGAGEYATIFTVLFFSAHRCAIAVENNYTISIDKTFDISSITRDWIELGEFPYPKAASNNETHFRCTEGAGKGLCAHTEFRVNGGLQSNPAAYWNGWMRMEPKEVNIGDGEKLIFSAYWKGSPSIRWEETNRRNMKSIFHQYTIGVASIGSSAGSITKWSSGEQGPSLRCGDLGGCTIDTHTYFDNRSGGALKLAVKLPAAFRKGTYTFSNVEVLDLGHTARNASGTTQQSVSAKVYISGKITVPERCYIDTGSGSEIKFNDVSAGASNGEIDKRSFTLKTTCKYINLKLNQYIKVNGSNGLSEYEIFSRDNSNEKALALVMRIVRGDNGEDFSANCEPDHSGRVYFSKEYLLREINGNGLNIHTYNDIIRFSLCKYGIPKDYGEKIIPLTIVSRWSDS

SEQ ID NO: 82 - protein, 278 aa, Escherichia coli
VLYYNNFDGGYCGQSLCFFRNSPTGYRRCAAGTVMDSYFLGGGTGMIALHSLAFGYSGQHPLGTLDGCFDTGSLTAIIGANGTGKSTLLKTLAGLLPPLGGCFCMVPQGQRQLGYLPQLTEFDRQFPLSVNDLVLMGCIPHSGMFGRISCLWRKKAIEALDTVGMTEFSQMHIGTLSGGQLQRVLFARLLVMQPSVILLDEPFTGIDVQTIRTLLVVIRQLHLEGRTILAVLHDMEQVEKYFSHVLMLSAEGHRWGRSADILHSLTAAVTPPQQGLLP

SEQ ID NO: 83 - protein, 292 aa, Escherichia coli
MKRSILVVALSSLLVSPLVIAKELNVVASFSVLGDMVSQIGGPYVHVTDLVQPDGDPHEFEPSPKDSKTLAQADVVFVNGLGLEGWLDRLMKASGYRGEVITASNGIDTLKMKEDGTTITDPHAWNSMKNGIVYAHNIVNGLSKADPEHASDYRKQGDSYIQQLQQLDNYATQTFAAIPREKRKVLTSHDAFGYFAAAYGVRFLSPVGYSTESEASSKNVAKLINQIKREHVKLYFIENQTDPRLVKQIANASGAQAGGELYPEALTDSSGLAATYTAAFKHNVDTLAAGMK

SEQ ID NO: 84 - protein, 271 aa, Escherichia coli
MKEITSPSSLNTFSPISISIRNIATNKFLIIHTDNCDLHIENEHGNFFCTRGSFVFMSRNMNFSCRIHKIETGRPPYKTIRLGREELTMLTNILHSTHVYCLNKHITEGKSHNGIVCIGGNKDWGYIFNKIETSKDSALKMLKLSYLISRMGIAPDIIISLMASTAVTFTEKIRNQIENNPSKRWCLNMIADEFNISEISVRKRLESEGTSFSNLFLKVRMEKSMQLLLQNELQICQISKSVGFRSTSYFIKIFKDYFGITPKQLIIYFRN

SEQ ID NO: 85 - protein, 205 aa, Escherichia coli
VCSVMKKRLLFMIKSVIAGAVAMAVVSFGVNAAATVPQGQGKVTFSGTVVDAPCGIDAQSADQSVDFGQISKVFLDNDGQTTPKAFDIKLVNCDITNYKKPATGAAPGGAPKAGTVSMTFSGVTAGAGNANDMLQTNGETNTAIVVIDPHGKRVKFDGRTSTGVSNLINGDNTIHFTAAVMKDGSGNHVKEGAFSAVANFNLTYQ

SEQ ID NO: 86 - protein, 568 aa, Escherichia coli
MNVEKLIVDHMETWTSALQTRSTAGRGSSGKIDLYGIKKLRELILELAVRGKLVPQDPNDEPASELLKRIAAEKAELVKQAKIKKQKPLPEISEEEKLFELPEGWEWVRFGNIYEMEYGNNLPQEKRSNSGEYNVYGSNGVVGTHNEACIKSPCIIIGRKGSAGALNLSNQPACWVTDVAYSTIPPIAMVLEFVFIQFHTLGLDKLGKGIKPGLNRNDAYSLVIAIPPRSEQKAIVSKVNELMSLCDQLEQQSLTSLDAHQQLVETLLGTLADSQNAEELAENWARISEHFDTLFTTEASVDALKQTILQLAVMGKLVPQDPNDEPASELLKRIAQEKAQLVKEGKIKKQKPLLPISDEEKPFELPNGWEWCRLGELIDSIDAGWSPACSSEPAAPGEWGVLKTTAVQSLEYREYENKALPKNKAPRPQLEVKAGDILITRAGPKNRVGISCLVENTRENLMISDKIIRFHLISEDISEKYISLCLNYGFTSTYLENSKSGMAESQMNISQDILKMAPIAIPTTHEQLKITDKINEMMDYFITLKSQIQSAQQTQLHLADALTNAAIN

SEQ ID NO: 87 - protein, 134 aa, Escherichia coli
MIRLIFSFLLCTLCAGCLSQPFHSCSIVMVNTCKVSISISTVNNSTHYDGKVRENFHLDVGERKPIVYFIYTGRESDVFNKNPVNIVSELSILISDSNNSKTLSGYHISSLLKKSSSSMESDFSFEINDPSICP

SEQ ID NO: 88 - protein, 129 aa, Escherichia coli
LQFQTVRRYKFAQNNQPELQYRQCTRPASAFALPSKSLLAQYVDVLSDVRTSRKQHVGWFETIQMVIYRLISLALTPRRSSSSIRGEHPSRRGNKALKRSLFLTAFAALIAPISKAYYTHKMRQGKRHN

SEQ ID NO: 89 - protein, 436 aa, Escherichia coli
MSGPKKIGLITCIAVVAGNMMGSGIALLPASLAKIGSVAIWGWIIAAIGALSLAYVFARLATNNPQTGGPIAYAGEVAPIFGHQTSILYYHANWIGNLAIAITAVAYLSVFFPVLNNPVPAAITSIAMVWLFTFVNLLGGAWVSRLTSIGLVLVLIPVVGTALFGWGSFDIEVYRQNWMAQPDGNVHQAVINSVLLCLWAFVGVESAAVSSDLVHNPSRTVPLATLGGTALAGIVYIAASQVIAGMFPNEVVANSGAPFALSASRIVGDWAAPFVSAFTAIACLTSLGSWMMLVGQAGRRAAMDGNFPAVFGETDNNGVPKKGLLIASTMMSILMVLLTILSASGSNAADLFTQLTSIAVLLTMFPYFYSAIDLIRFESATSKSILSLIASAFAMLFCFAALAGAEHYEITATIIISLLIFAFYARKLGGIQHKNI

SEQ ID NO: 90 - protein, 404 aa, Escherichia coli
MPVRLNTIPDAEPYPAPPAHIKWLSALLLMLSVGVALTSLFASDELAKNGPHFWGLACGVPAFIWSLVASVRWLFFITQYIRADAWNKRREEVILQETRRGRRALQILSFSVQTALNGDSVAETTTAFLARQQVLNTYADLRGEDTVRRSVIPVLTNKPVTGRLSEIISRLFFDIRPQLFLLPPDFHINVLLEIDAPLSGASVRTIWQDEWQKAGLPEARLFSAPEPGLAAVDDWLDNFIQEKAVLLVISVRLEPKNPERTAESATALLLANRLTQTALTPLALLHRPERITDTEMMASGIAQALDWMPVQPDAISGMWTAELDREQRAALLSLNQPFAQEALMYELDAFLGRSGPAAPWLSVAAATLAAIQSQHPQLTLSGVQGGHYSWATVVSPFVSPQEAS

SEQ ID NO: 91 - protein, 208 aa, Escherichia coli
MRRFCTVLVLLSMAYFIIGGLFAYLNKLELPIYLNGMAIVGFVMTIVCIYSLSKPAITQSDLEQLEATSLESITKTISELKSLKTNKNSTQEEILDLEKKRKEMELLVKKASMALFFREQLNYHERRILSEIKGSETLNHSLSEIKEIKGKLKALDEEIACDPEVEFLRSILLEASRRTPTLDELTEGMPFIFRIMARSTQLILGNKI

SEQ ID NO: 92 - protein, 258 aa, Escherichia coli
MARSGFEVQKAAVHALFLRELRTRFGKYRLGYLWAVLEPAAHLLIMLAIFGFFMHRTMPDISFPVFLINGIIPYFIFSNIATRSIGAIEANQGLFNYRPVRPIDTIIARAILEVLIYSIVYLVLMSLLLIIGEQFKIYNILLLFSAWILLALFSCGIGLIFMVIGKTFPETEKFLPIILKPLYFVSCIMLPLHAIPKGYWGYILWNPIVHVVELCRESVVTGYVSEGVSLYYLFISTLILLFLGLALYSSREEYMLTS

SEQ ID NO: 93 - protein, 308 aa, Escherichia coli
MGEKCRYTPFPHGCSHATAAGAAAMMLLHLLCEPFGDFGFMRRALVGCLALTLSAAPLGCFLLLRRMSLIGDALSHAVLPGVAIGYLVSGMSLVAMGVGGFIAGLSVAMLSGVVSRRTGLREDASFAGFYLGSLALGVTLVSLRGSSVDLLHVLFGSILAIDANALITIGIISSGSVLVLALIYRVLVIESFDVTFLKVLSRRSRALIHCLFLSMVVLNLVAGFQLLGTLMTVGIMMLPAASARFWSQRLSIMLLAAVGIGTCASLVGLTWSYYADLPAGPAVILTSTLFFCFSVLFGSNGGMLCRCR

SEQ ID NO: 94 - protein, 142 aa, Escherichia coli
MINITTKDGTQVSGEVRDLVILLETDGKTVSDYLQPIPPKKDTHWSMIFIPVAIYILLNLVYIIAHPLFPEKYLKTSTSIFLFISFILSAIVAYFVFHKHKSATLSICSLVFLVLIVGANVGFISYSDLTQKATQKLDSLSK

SEQ ID NO: 95 - protein, 401 aa, Escherichia coli
MNQSEQRKKILVLTPRFPYPVIGGDRLRVYMLCKELSKKYDLILLSLCDQPLELEININDSVFKEIHRVYLPKYKSYYNVLKALVTQKPLQIAYYQSDTFKNKYNKLIKQCDAVFCHLIRVADYVKDTDKFKILDMTDAISLNYSRVKKLASKKSLRAIIYSLEQKRLESYERSVANLFDLTTFISSVDRDYLYPNPGSNIHIVNNGVDTSALRYIKREIKIDKPVELIFIGNMYSLQNMDAAKHFAKNILPCLYDEFNIIFKVIGKISETNKNILNSFKNTIALGTVDDINSSASTGHIGICPVRLGAGVQNKILEYMALGLPCITSSIGYEGINAKSGSEIFVADTVEQYKNVLREIIYDYNRYTEVAENARSFVENNFSWESKVANLMNTLDEKLYEQ

SEQ ID NO: 96 - protein, 129 aa, Escherichia coli
MGGAVNQTTINNGVLQVYGAATDPTIKGGRLIVEKDGIAVFAVIEKGGLLEVKATGDFKIFVTDTGASPAAGDNLTLVTTGAVMRHLRWAMPEALLISVRMNIPCWIMATIAGVSQRIAYKCPLQPRMC

SEQ ID NO: 97 - protein, 2683 aa, Escherichia coli
MSLTLNKILVLCALLISAMLPGWSWAESAWQDSSDTVGEFNGTVPTADSASIPVYQGSVFLDPAKTHDVAFTAKPSEFSADVSVSKLLVTNPQDREGDIIATPRWENQTPPAISLVWADAATPGTLLDPQPVADRSFCAQGLAGRSLVAWAQPDPQQTMPLLYLLTSTGYPYESVLTLADQKVTLKIAPAQGDLISVSAAGYDESSGAAKMTVGGSITLTVTTKDCVGNVVGNIPFVIKRKDAENRQGVVNNTAPVKLGTTELTTTATEYRGTTDANGVATVTVTQANGPGVKTPLVASLAGIAQASETAVIFTVLTSPDVPQATMWGHMPDTLKARDYTFSRPKLAAEVDNEDGTVNDHNETWSTFTWSGADKHCDILPGMRQFGALATVVPTSVQDVAGWPMQGNFYWSSLAGMSGQHHAADVSNRSEAQKPDDTTFIVSCVDKEAPDVEPKLVLTPGSYDSTIKAMKVKVGEEASLRLTITDSKNNDQPLAYYYFSLHLDDGINRKNQTDAAWETHPVQIDGGSNVRKVDAHTYEGITDANGEATLTLTQPGGVGVKTHITARMRSDFTASDEKDVIFTVITSPDTDKARMWGHMLGIIEANNIFKRPRLADETDNELGSVRENNEDWALFDQNSSMQAECGLGHIPSQSSLHSLFAAHPANAIGTEYGWPTLQKAYLSAVEETSHASVNLATGNIDTYSGFKQNYLSCSGNEMVAKIAATTDRDVSAGSRAQAKVGDTITMTVRTFNALNNAPVPYTAFTITKDMGKNRQGQTTGFDDPTRGAIEMNGTLYGTSQPSLVYAGTTDAQGFATVEIKQSQGVGLSTPLNIVPVNSYIPNTVNYNVIFTTLTSPDAVGAQMWGHMDETITVDALTFARPRLAAEVFSPDGTLTENNEVWSRVSQANASSTSKGGCGANMLPRRSQLSALYDANNGNGVQTVHGWPTQRQPYWSSSPADQVPHYYTIALNDGARTVGGSTAVYVSCLTTANNPASSITLEVVDPAQWNAAANAAKLKKGETLQVKVTVKDAQGNPLGDIPFTLKRGDGYTRSEEKHVAGSGDALVAPVVVNGGLADETSLNNTAAAYSAITGSDGTKILTITRPDTHGTKTSLTATLYSDTTKKATLDTIFTVVTSPDSDKAKMWGHMPETVTAADGTVFKRPLLLKELSSTSGRTAIAEENEDWAQFTQTQAISTSSNGCGSEYVPSQAGLESLYEANRGNAMKTVQGWPVASSYLSSTTGSSSLEQRDFKAVNLSSGTSSIIPSATKELLTCQTTPIVKASQIVLEAADPTKFDSTNNVVKAKKGEEAVLRVTTKDAQGNPVGNTAFTLKRNTSVNRANVSTTTSIASLAVTDAWGNTQDDFLSTTLVIYGVTGADGITTFTLKQDQTTGLKTELTAALDSSSSTKSTLPVVFTVLTSPDSPKAKFWGHMAETATGDDGLIYRRPLLRDENSATTSIGTLVEEGEAWSTFPSGQANDTSINGCGAEYVPTDNELRAIYAHQGSSALHDAIGWPVSRFYISNTVADTFTQTFTYDVVSLKTGDETQMPSSGGALLSCRTTPVAVASQIIVEANDTAQFVKVDDTLSALKVKKGEDAVIRVVTKNAQGNSVPNVPFILRREGSKNRQNAEMINKSITVINAAGASARMNSSSSLLYGVTGADGTTSFTVKQDDSMGLVTNMYAQLYQLTIESNKLPVMFTVITSPDTPLASYWGHMSETFTTRSGIAFKRPLLTAEHPAGQSTMANNESWLSLNTAAKNDVSKSDCGEPYQPLLSEFQELYSEHPNGAIGTDLGLPLTNTWWAYDKIAYANVWYDQSINLSNGSSSRALSNTVAFVSCLVNPHAVAASIKMTSTALDAEKTASNDGRPSATAAKGTAIRMTVIVRDSGGNLLPGANFNLIRGTALDRAKNRLDSTYDDLTIVPVTPAGVNMSLYNNGAQALLTTGSDGKATFDVTQNETYGLATPLTATLMRDTTKSATMDVIFTVITSPNSPKAKYWGHMPDTFTSRAGVTFKRPLLAAEATLGSSVSNNNESWSYLFYTNKVTPDCPVEYQPRLNELQGLYNDHPGGTILTDLGLPITAGSGNWWTYEMSTTDALTWYYGVINLKTGQSTTTINGYALMLCLTQPHSAPASLTLSSTAYDEGRTASNGGTPTSSVKKGEMLPIVVTIKDANGNPVGGEGVTLKRVQAKSRSGISVSSNTVDDLILDEVTPTSARISFNQNTSAWSGFTGSDGTITFNVTQNNTVGLVTPFTASLARNPQVTANQDLIFTVVTSPDSAKANYWGHMPATLTAVNGAVFERPKLWSELTSTSGVGKINNNNEDWPYFTPTQKSDASVSPCEVARQPLFNDLSSLSARYPNNTFVTETGWPAYYTWWAEDKSADGKDQSVDLRNGTLYTGSTKSFQPCLANARSTVSSVTLTSTAFDADSQAAKVKKGEAMSVTVTVKDSAGNTVPNVEFTLKRGEASPRNAGATLYGNVVAMDDLIVQPLSGSAITLSESGNTISGMTGADGTASFSLRQDNTPGYKMPLTVTLANYASATDTLDAIFTVPTSPNVSSAHFWGHMADTVVVNSKSLHRPLLTTELPSGANPVSSPIINYENWASAHIIDASKWDIARQCGSIENAPTYNELELLHTVFNSLGWPSSPSFPYLSSQQCGMDEGTGAQDCSITLINKPGLVTCFQ

SEQ ID NO: 98 - protein, 193 aa, Escherichia coli
MVVNKTTAVLYLIALSLSGFIHTFLRAEERGIYDDVFTADELRHYRINERGGRTGSLAISGALLSSPCTLVSNEVPLSLRPENHSAAAGAPLMLRLAGCGDGGALQPGKRGVAMTVSGSLVTGPGSGSALLPDRKLSGCDHLVIHDGDTFLLCRPDRRQEEMLAAWRKRATQEGEYSDARSNPAMLRLSIKYE

SEQ ID NO: 99 - protein, 159 aa, Escherichia coli
MMTLEADSVNVQALDMGRIVIDVDGVTLAELINVVCDNGYSLRVVDESDRASTDCTPPFAVLTGIRCSTAHITAKDNAWLYSLSHQTNDTGESEWIHFTGSGYLLRTDARSYPVLRLKRLGLSKTFRRLIATLIRRYGVSLIHLDASAGCLPGLPTFDW

SEQ ID NO: 100 - protein, 464 aa, Escherichia coli
MKKSTLSLAIGLLLACSTGMAKTQHLTLEQRMALLEERLEAAEMRAAKAESQVKQLQTQQAAEIREIKAAQGNTPVNGQATAESAKKNSTSPNLLLSGYGDLKIYGDVEFNMDAESNHGLLAMTNADVNSDPTNEQWNLNGRILLGFDGMRKLDNGYFAGFSAQPLGDMHGSVNIDDAVFFFGKENDWKVKVGRFEAYDMFPLNQDTFVEHSGNTANDLYDDGSGYIYMMKEGRGRSNAGGNFLVSKQLDNWYFELNTLLEDGTSLYNDGNYHGRDMEQQKNVAYLRPVIAWSPTEEFTVSAAMEANVVNNAYGYTDSKGNFVDQSDRTGYGMSMTWNGLKTDPENGIVVNLNTAYLDANNEKDFTAGINALWKRFELGYIYAHNKIDEFSGVVCDNDCWIDDEGTYNIHTIHASYQFANVMDMENFNIYLGTYYSILDSDGDKIHGDDSDDRYGARVRFKYFF

SEQ ID NO: 101 - protein, 810 aa, Escherichia coli
MAMKKLLIASLLFSSATVYGAEGFVVKDIHFEGLQRVAVGAALLSMPVRTGDTVNDEDISNTIRALFATGNFEDVRVLRDGDTLLVQVKERPTIASITFSGNKSVKDDMLKQNLEASGVRVGESLDRTTIADIEKGLEDFYYSVGKYSASVKAVVTPLPRNRVDLKLVFQEGVSAEIQQINIVGNHAFTTDELISHFQLRDEVPWWNVVGDRKYQKQKLAGDLETLRSYYLDRGYARFNIDSTQVSLTPDKKGIYVTVNITEGDQYKLSGVEVSGNLAGHSAEIEQLTKIEPGELYNGTKVTKMEDDIKKLLGRYGYAYPRVQSMPEINDADKTVKLRVNVDAGNRFYVRKIRFEGNDTSKDAVLRREMRQMEGAWLGSDLVDQGKERLNRLGFFETVDTDTQRVPGSPDQVDVVYKVKERNTGSFNFGIGYGTESGVSFQAGVQQDNWLGTGYAVGINGTKNDYQTYAELSVTNPYFTVDGVSLGGRLFYNDFQADDADLSDYTNKSYGTDVTLGFPINEYNSLRAGLGYVHNSLSNMQPQVAMWRYLYSMGEHPSTSDQDNSFKTDDFTFNYGWTYNKLDRGYFPTDGSRVNLTGKVTIPGSDNEYYKVTLDTATYVPIDDDHKWVVLGRTRWGYGDGLGGKEMPFYENFYAGGSSTVRGFQSNTIGPKAVYFPHQASNYDPDYDYECATQDGAKDLCKSDDAVGGNAMAVASLEFITPTPFISDKYANSVRTSFFWDMGTVWDTNWDSSQYSGYPDYSDPSNIRMSAGIALQWMSPLGPLVFSYAQPFKKYDGDKAEQFQFNIGKTW

SEQ ID NO: 102 - protein, 346 aa, Escherichia coli
MKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDVYTRLGGMVWRADTKSNFDGKNHDTGVSPVFAGGVEYAITPEIATRLEYQWTNNIGDAHTIGTRPDNGMLSLGVSYRFGQGEAAPVVAPAPAPAPEVQTKHFTLKSDVLFTFNKATLKPEGQAALDQLYSQLSNLDPKDGSVVVLGYTDRIGSDAYNQALSERRAQSVVDYLISKGIPADKISARGMGESNPVTGNTCDNVKQRAALIDCLAPDRRVEIEVKGIKDVVTQPQA

SEQ ID NO: 103 - protein, 219 aa, Escherichia coli
MKKRVYLIAAVVSGALAVSGCTTNPYTGEREAGKSAIGAGLGSLVGAGIGALSSSKKDRGKGALIGAAAGAALGGGVGYYMDVQEAKLRDKMRGTGVSVTRSGDNIILNMPNNVTFDSSSATLKPAGANTLTGVAMVLKEYPKTAVNVIGYTDSTGGHDLNMRLSQQRADSVASALITQGVDASRIRTQGLGPANPIASNSTAEGKAQNRRVEITLSPL

SEQ ID NO: 104 - protein, 394 aa, Escherichia coli
VSKEKFERTKPHVNVGTIGHVDHGKTTLTAAITTVLAKTYGGAARAFDQIDNAPEEKARGITINTSHVEYDTPTRHYAHVDCPGHADYVKNMITGAAQMDGAILVVAATDGPMPQTREHILLGRQVGVPYIIVFLNKCDMVDDEELLELVEMEVRELLSQYDFPGDDTPIVRGSALKALEGDAEWEAKILELAGFLDSYIPEPERAIDKPFLLPIEDVFSISGRGTVVTGRVERGIIKVGEEVEIVGIKETQKSTCTGVEMFRKLLDEGRAGENVGVLLRGIKREEIERGQVLAKPGTIKPHTKFESEVYILSKDEGGRHTPFFKGYRPQFYFRTTDVTGTIELPEGVEMVMPGDNIKMVVTLIHPIAMDDGLRFAIREGGRTVGAGVVAKVLG

SEQ ID NO: 105 - protein, 747 aa, Escherichia coli
MAPSKTAQPKHSLRKIAVVVATAVSGMSVYAQAAVEPKEDTITVTAAPAPQESAWGPAATIAARQSATGTKTDTPIQKVPQSISVVTAEEMALHQPKSVKEALSYTPGVSVGTRGASNTYDHLIIRGFAAEGQSQNNYLNGLKLQGNFYNDAVIDPYMLERAEIMRGPVSVLYGKSSPGGLLNMVSKRPTTEPLKEVQFKAGTDSLFQTGFDFSDALDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLDLYNPVNTDFDFNAKDPDNSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKQYEVGVKYVPEDRPIVVTGAVYNLTKTNNLMADPEGSFFSVEGGEIRARGVEIEAKAALSASVNVVGSYTYTDAEYTTDTTYKGNTPAQVPKHMASLWADYTFFDGPLSGLTLGTGGRYTGSSYGDPANSFKVGSYTVVDALVRYDLARVGMAGSNVALHVNNLFDREYVASCFNTYGCFWGAERQVVATATFRF

SEQ ID NO: 106 - protein, 171 aa, Escherichia coli
MKKIACLSALAAVLAFTAGTSVAATSTVTGGYAQSDAQGQMNKMGGFNLKYRYEEDNSPLGVIGSFTYTEKSRTASSGDYNKNQYYGITAGPAYRINDWASIYGVVGVGYGKFQTTEYPTYKHDTSDYGFSYGAGLQFNPMENVALDFSYEQSRIRSVDVGTWIAGVGYRF

SEQ ID NO: 107 - protein, 360 aa, Escherichia coli
MKKLTVAISAVAASVLMAMSAQAAEIYNKDSNKLDLYGKVNAKHYFSSNDADDGDTTYARLGFKGETQINDQLTGFGQWEYEFKGNRAESQGSSKDKTRLAFAGLKFGDYGSIDYGRNYGVAYDIGAWTDVLPEFGGDTWTQTDVFMTGRTTGVATYRNNDFFGLVDGLNFAAQYQGKNDRTDVTEANGDGFGFSTTYEYEGFGVGATYAKSDRTDGQVAYGKSKFNASGKNAEVWAAGLKYDANNIYLATTYSETQNMTVFGNNHIANKAQNFEAVAQYQFDFGLRPSVAYLQSKGKDLGVHGDRDLVKYVDVGATYYFNKNMSTFVDYKINLIDDSKFTKTAGIDTDDIVAVGLVYQF

SEQ ID NO: 108 - protein, 245 aa, Escherichia coli
MTRMKYLVAAATLSLFLAGCSGSKEEVPDNPPNEIYATAQQKLQDGNWRQAITQLEALDNRYPFGPYSQQVQLDLIYAYYKNADLPLAQAAIDRFIRLNPTHPNIDYVMYMRGLTNMALDDSALQRFFGVDRSDRDPQHARAAFSDFSKLVRGYPNSQYTTDATKRLVFLKDRLAKYEYSVAEYYTERGAWVAVVNRVEGMLRDYPDTQATRDALPLMENAYRQMQMNAQAEKVAKIIAANSSNT

SEQ ID NO: 109 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNTLLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 110 - protein, 236 aa, Escherichia coli
MVKKAIVTAMAVISLFTLMGCNNRAEVDTLSPAQAAELKPMPQSWRGVLPCADCEGIETSLFLEKDGTWVMNERYLGAREEPSSFASYGTWARTADKLVLTDSKGEKSYYRAKGDALEMLDREGNPIESQFNYTLEPAQSSLPMTPMTLRGMYFYMADAATFTDCATGKRFMVANNAELERGYLAARGNSEKPVLLSVEGHFTLEANPDTGAPTKVLAPDTAGKFYPNQDCSSLGL

SEQ ID NO: 111 - protein, 173 aa, Escherichia coli
MQLNKVLKGLMIALPVMAIAACSSNKNASNDGSEGMLGAGTGMDANGGNGNMSSEEQARLQMQQLQQNNIVYFDLDKYDIRSDFAQMLDAHANFLRSNPSYKVTVEGHADERGTPEYNISLGERRANAVKMYLQGKGVSADQISIVSYGKEKPAVLGHDEAAYAKNRRAVLVY

SEQ ID NO: 112 - protein, 332 aa, Escherichia coli
MNKKVLTLSAVMASMLFGAAAHAADTRIGVTIYKYDDNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNLADGKGAADGTNWKIDNKVVRVPYVGVDKDNLAEFSKK

SEQ ID NO: 113 - protein, 375 aa, Escherichia coli
MKVKVLSLLVPALLVAGAANAAEVYNKDGNKLDLYGKVDGLHYFSDDKSVDGDQTYMRLGFKGETQVTDQLTGYGQWEYQIQGNAPESENNSWTRVAFAGLKFQDIGSFDYGRNYGVVYDVTSWTDVLPEFGGDTYGSDNFMQQRGNGFATYRNTDFFGLVDGLNFAVQYQGQNGSVSGENDPDFTGHGITNNGRKALRQNGDGVGGSITYDYEGFGVGAAVSSSKRTDAQNTAAYIGNGDRAETYTGGLKYDANNIYLAAQYTQTYNATRVGSLGWANKAQNFEAVAQYQFDFGLRPSVAYLQSKGKNLGTIGTRNYDDEDILKYVDVGATYYFNKNMSTYVDYKINLLDDNQFTRDAGINTDNIVALGLVYQF

SEQ ID NO: 114 - protein, 1157 aa, Escherichia coli
MRKFTLNIFTLSLGLAVMPMVEAAPTAQQQLLEQVRLGEATHREDLVQQSLYRLELIDPNNPDVIAARFRSLLRQGDIDGAQKQLDRLSQLAPSSNAYKSSRTTMLLSTSDGRQALQQARLQATTGHAEEAVASYNKLFNGAPPEGDIAVEYWSTVAKIPARRGEAINQLKRINADTPGNTGLQNNLALLLFSSDRRDEGFAVLEQMAKSNAGREGASKIWYGQIKDMPVSDASVQALKKYLSIFSDGDSVAAAQSQLAEQQKQLADPAFRARAQGLAAVDSGMAGKAIPELQQAVRANPKDSEALGALGQAYSQKGDRANAVANLEKALALDPHNSNNDKWNSLLKVNRYWLAIQQGDAALKANNPDRAERLFQQARNVDNTDSYAVLGLGDVAMARKDYPAAERYYQQTLRMDSGNTNAVRGLANIYRQQSPEKAEAYIASLSASQRRSIDDIERSLQNDRLAQQAEALENQGKWAQTAALQRQRLALDPGSVWITYRLSQDLWQAGQRSQADTLMRNLAQQKPNDPEQVYAYGLYLSGHDQDRAALAHINSLPHAQWNSNIQELVNRLQNDQVLETANRLRENGKEAEAEAMLRQQPPSSRIDLTLADWAQQRRDYTAARAAYQNVLTREPTNADAILGLTEVDIAAGDKAAARSQLAKLPATDNASLNTQRRVALAQAQLGDTAAAQQTFNKLIPQAKSQPPSMESAMVLRDGAKFEAQAGDPTQALETYKDAMVASGVTTTRPQDNDTFTRLTRNDDKDDWLKRGVRSDAADLYRQQDLNVTLEHDYWGSSGTGGYSDLKAHTTMLQVDAPYSDGRMFFRSDFVNMNVGSFSTNADGKWDDNWGTCTLQDCSGNRSQSDSGASVAVGWRNDVWSWDIGTTPMGFNVVDVVGGISYSDDIGPLGYTVNAHRRPISSSLLAFGGQKDSPSNTGKKWGGVRADGVGLSLSYDKGEANGVWASLSGDQLTGKNVEDNWRVRWMTGYYYKVINQNNRRVTIGLNNMIWHYDKDLSGYSLGQGGYYSPQEYLSFAIPVMWRERTENWSWELGASGSWSHSRTKTMPRYPLMNLIPTDWQEEAARQSNDGGSSQGFGYTARALLERRVTSNWFVGMAIDIQQAKDYAPSHFLLYVRYSAAGWQGDMDLPPQPLIPYADW

SEQ ID NO: 115 - protein, 562 aa, Escherichia coli
MSGEKKAKGWRFYGLVGLGAIVLLSTGVWALQYAGSGPEKTLSPLVVHNNLQIDLNEPDLFLDSDSLSQLPKDLLTIPFLHDVLSEDFVFYYQNHADRLGIEGSIRRIVYEHDLTLKDKLFSSLLDQPAQAALWHDKQGHLSHYMVLIQRSGLSKLLEPLLFAATSDSQLSKTEISSIKLNSETIPVYQLRYNGNNALMFATYQDKMLVFSSTDMLFKDDQQDTEAAAIASDLLSGKKRWETSFGLEERTAEKTPVRQRIVVSARLLGFGYQRLVPSFAGMRFEMGNDGWHSFLALNDESASVDASFDFTPVWNSMPAGASFCVAVPYSHGIAEEMLSHISQENDKLNGALDGGAGLCWYEDSKLQTPLFVGQFDGTAEQAQLPGKLFTQNIGAHESKAPEGVLPVSQTQQGEAQIWRREVSSRYGQYPKAQAAQPDQLMSDYFFRVSLAMQNKTLLFSLDDTLVNNALQALNKTRPAMVDVIPTDGIVPLYINPQGMAKLLRNETLTSLPKNLEPVFYNAAQTLLMPKLDALSQQPRYVMKLAQMEPGAAWQWLPITWQPL

SEQ ID NO: 116 - protein, 474 aa, Escherichia coli
MKKTTLALSALALSLGLALSPLSATAAETSSATTAQQMPSLAPMLEKVMPSVVSINVEGSTTVNTPRMPRNFQQFFGDDSPFCQEGSPFQSSPFCQGGQGGNGGGQQQKFMALGSGVIIDADKGYVVTNNHVVDNATVIKVQLSDGRKFDAKMVGKDPRSDIALIQIQNPKNLTAIKMADSDALRVGDYTVAIGNPFGLGETVTSGIVSALGRSGLNAENYENFIQTDAAINRGNSGGALVNLNGELIGINTAILAPDGGNIGIGFAIPSNMVKNLTSQMVEYGQVKRGELGIMGTELNSDLAKAMKVDAQRGAFVSQVLPNSSAAKAGIKAGDVITSLNGKPISSFAALRAQVGTMPVGSKLTLGLLRDGKQVNVNLELQQSSQNQVDSSTIFNGIEGAEMSNKGKDQGVVVNNVKTGTPAAQIGLKKGDVIIGANQQAVKNIAELRKVLDSKPSVLALNIQRGDSTIYLLMQ

SEQ ID NO: 117 - protein, 344 aa, Escherichia coli
MAYSVQKSRLAKVAGVSLVLLLAACSSDSRYKRQVSGDEAYLEAAPLAELHAPAGMILPVTSGDYAIPVTNGSGAVGKALDIRPPAQPLALVSGARTQFTGDTASLLVENGRGNTLWPQVVSVLQAKNYTITQRDDAGQTLTTDWVQWNRLDEDEQYRGRYQISVKPQGYQQAVTVKLLNLEQAGKPVADAASMQRYSTEMMNVISAGLDKSATDAANAAQNRASTTMDVQSAADDTGLPMLVVRGPFNVVWQRLPAALEKVGMKVTDSTRSQGNMAVTYKPLSDSEWHELGASDPGLASGDYKLQVGDLDNRSSLQFIDPKGHTLTQSQNDALVAVFQAAFSK

SEQ ID NO: 118 - protein, 420 aa, Escherichia coli
MIKATDRKLVVGLEIGTAKVAALVGEVLPDGMVNIIGVGSCPSRGMDKGGVNDLESVVKCVQRAIDQAELMADCQISSVYLALSGKHISCQNEIGMVPISEEEVTQEDVENVVHTAKSVRVRDEHRVLHVIPQEYAIDYQEGIKNPVGLSGVRMQAKVHLITCHNDMAKNIVKAVERCGLKVDQLIFAGLASSYSVLTEDERELGVCVVDIGGGTMDIAVYTGGALRHTKVIPYAGNVVTSDIAYAFGTPPSDAEAIKVRHGCALGSIVGKDESVEVPSVGGRPPRSLQRQTLAEVIEPRYTELLNLVNEEILQLQEKLRQQGVKHHLAAGIVLTGGAAQIEGLAACAQRVFHTQVRIGAPLNITGLTDYAQEPYYSTAVGLLHYGKESHLNGEAEVEKRVTASVGSWIKRLNSWLRKEF

SEQ ID NO: 119 - protein, 161 aa, Escherichia coli
VKKWLLAAGLGLALATSAQAADKIAIVNMGSLFQQVAQKTGVSNTLENEFKGRASELQRMETDLQAKMKKLQSMKAGSDRTKLEKDVMAQRQTFAQKAQAFEQDRARRSNEERGKLVTRIQTAVKSVANSQDIDLVVDANAVAYNSSDVKDITADVLKQVK

SEQ ID NO: 120 - protein, 213 aa, Escherichia coli
MTKMSRYALITALAMFLAGCVGQREPAPVEEVKPAPEQPAEPQQPVPTVPSVPTIPQQPGPIEHEDQTAPPAPHIRHYDWNGAMQPMVSKMLGADGVTAGSVLLVDSVNNRTNGSLNAAEATETLRNALANNGKFTLVSAQQLSMAKQQLGLSPQDSLGTRSKAIGIARNVGAHYVLYSSASGNVNAPTLQMQLMLVQTGEIIWSGKGAVSQQ

SEQ ID NO: 121 - protein, 392 aa, Escherichia coli
MQLRKLLLPGLLSVTLLSGCSLFNSEEDVVKMSPLPTVENQFTPTTVWSTSVGSGIGNFYSNLHPALADNVVYAADRAGLVKALNADDGKEIWSVNLAEKDGWFSKDPALLSGGVTVSGGHVYIGTEKAQVYALNTSDGTVAWQTKVAGEALSRPVVSDGLVLIHTSNGQLQALNEADGAVKWTVNLDMPSLSLRGESAPATAFGAAVVGGDNGRVSAVLMEQGQMIWQQRISQATGSTEIDRLSDVDTTPVVVNGVVFALAYNGNLTALDLRSGQIMWKRELGSVNDFIVDGNRIYLVDQNDRVMALTIDGGVTLWTQSDLLHRLLTSPVLYNGNLVVGDSEGYLHWINVEDGRFVAQQKVDSSGFQTEPVAADGKLLIQAKDGTVYSITR

SEQ ID NO: 122 - protein, 678 aa, Escherichia coli
MVPSTFSRLKAARCLPVVLAALIFAGCGTHTPDQSTAYMQGTAQADSAFYLQQMQQSSDDTRINWQLLAIRALVKEGKTGQAVELFNQLPQELNDSQRREKTLLAAEIKLAQKDFAGAQNLLAKITPADLEQNQQARYWQAKIDASQGRPSIDLLRALIAQEPLLGAKEKQQNIDATWQALSSMTQEQANTLVINADENILQGWLDLQRVWFDNRNDPDMMKAGIADWQKRYPNNPGAKMLPTQLVNVKAFKPASTNKIALLLPLNGQAAVFGRTIQQGFEAAKNIGTQPVAAQVAAAPAADVAEQPQPQTVDGVASPAQASVSDLTGEQPAAQSVPVSAPATSTAAVSAPANPSAELKIYDTSSQPLSQILSQVQQDGASIVVGPLLKNNVEELLKSNTPLNVLALNQPENIENRVNICYFALSPEDEARDAARHIRDQGKQAPLVLIPRSSLGDRVANAFAQEWQKLGGGPVLQQKFGSTSELRAGVNGGSGIALTGSPITPRETTDSGMTTNNPTLQTTPTDDQFTNNGGRVDAVYIVATPGEIAFIKPMIAMRNGSQSGATLYASSRSAQGTAGPDFRLEMEGLQYSEIPMLAGGNLPLMQQALSAVNNDYSLARMYAMGVDAWSLANHFSQMRQVQGFEINGNTGSLTANPDCVINRKLSWLQYQQGQVVPAS

SEQ ID NO: 123 - protein, 191 aa, Escherichia coli
MKALSPIAVLISALLLQGCVAAAVVGTAAVGTKAATDPRSVGTQVDDGTLEVRVNSALSKDEQIKKEARINVTAYQGKVLLVGQSPNAELSARAKQIAMGVDGANEVYNEIRQGQPIGLGEASNDTWITTKVRSQLLTSGLVKSSNVKVTTENGEVFLMGLVTEREAKAAADIASRVSGVKRVTTAFTFIK

SEQ ID NO: 124 - protein, 270 aa, Escherichia coli
MKSLFKVTLLPTTMAVVLHAPITFAAEAAKPATTADSKAAFKNDDQKSAYALGASLGRYMENSLKEQEKLGIKLDKDQLIAGVQDAFADKSKLSDQEIEQTLQAFEARVKSSAQAKMEKDAADNEAKGKEYREKFAKEKGVKTSSTGLVYQVVEAGKGEAPKDSDTVVVNYKGTLIDGKEFDNSYTRGEPLSFRLDGVIPGWTEGLKNIKKGGKIKLVIPPELAYGKAGVPGIPPNSTLVFDVELLDVKPAPKADAKPEADAKAADSAKK

SEQ ID NO: 125 - protein, 427 aa, Escherichia coli
VNTFSVSRLALTLAFGVTLTACSSTPPDQRPSDQTAPGTSSRPILSAKEAQNFDAQHYFASLTPGAAAWNPSPITLPAQPDFVVGPAGTQGVTHTTIQAAVDAAIIKRTNKRQYIAVMPGEYQGTVYVPAAPGGITLYGTGEKPIDVKIGLSLDGGMSPADWRHDVNPRGKYMPGKPAWYMYDSCQSKRSDSIGVLCSAVFWSQNNGLQLQNLTIENTLGDSVDAGNHPAVALRTDGDQVQINNVNILGRQNTFFVTNSGVQNRLETNRQPRTLVTNSYIEGDVDIVSGRGAVVFDNTEFRVVNSRTQQEAYVFAPATLSNIYYGFLAVNSRFNASGDGVAQLGRSLDVDANTNGQVVIRDSAINEGFNTAKPWADAVISNRPFAGNTGSVDDNDEVQRNLNDTNYNRMWEYNNRGVGSKVVAEAKK

SEQ ID NO: 126 - protein, 700 aa, Escherichia coli
MANGWTGNILRVNLTTGNITLEDSSKFKSFVGGMGFGYKIMYDEVPPGTKPFDEANKLVFATGPLTGSGAPCSSRVNITSLSTFTKGNLVVDAHMGGFFAAQMKFAGYDVIIIEGKAKSPVWLKIKDDKVSLEKADFLWGKGTRATTEEICRLTSPETCVAAIGQAGENLVPLSGMLNSRNHSGGAGTGAIMGSKNLKAIAVEGTKGVNIADRQEMKRLNDYMMTELIGANNNHVVPSTPQSWAEYSDPKSRWTARKGLFWGAAEGGPIETGEIPPGNQNTVGFRTYKSVFDLGPAAEKYTVKMSGCHSCPIRCMTQMNIPRVKEFGVPSTGGNTCVANFVHTTIFPNGPKDFEDKDDGRVIGNLVGLNLFDDYGLWCNYGQLHRDFTYCYSKGVFKRVLPAEEYAEIRWDQLEAGDVNFIKDFYYRLAHRVGELSHLADGSYAIAERWNLGEEYWGYAKNKLWSPFGYPVHHANEASAQVGSIVNCMFNRDCMTHTHINFIGSGLPLKLQREVAKELFGSEDAYDETKNYTPINDAKIKYAKWSLLRVCLHNAVTLCNWVWPMTVSPLKSRNYRGDLALEAKFFKAITGEDMTQEKLDLAAERIFTLHRAYTVKLMQTKDMRNEHDLICSWVFDKDPQIPVFTEGTDKMDRDDMHASLTMFYKEMGWDPQLGCPTRETLQRLGLEDIAADLAAHNLLPA

SEQ ID NO: 127 - protein, 270 aa, Escherichia coli
MARIIVVTSGKGGVGKTTSSAAIATGLAQKGKKTVVIDFDIGLRNLDLIMGCERRVVYDFVNVIQGDATLNQALIKDKRTENLYILPASQTRDKDALTREGVAKVLDDLKAMDFEFIVCDSPAGIETGALMALYFADEAIITTNPEVSSVRDSDRILGILASKSRRAENGEEPIKEHLLLTRYNPGRVSRGDMLSMEDVLEILRIKLVGVIPEDQSVLRASNQGEPVILDINADAGKAYADTVERLLGEERPFRFIEEEKKGFLKRLFGG

SEQ ID NO: 128 - protein, 155 aa, Escherichia coli
MIKRVLVVSMVGLSLVGCVNNDTLSGDVYTASEAKQVQNVSYGTIVNVRQVQIQGGDDSNVIGAIGGAVLGGFLGNTIGGGTGRSLATAAGAVAGGVAGQGVQSAMNKTQGVELEIRKDDGNTIMVVQKQGNTHFSPGQRVVLASNGSQVTVSPR

SEQ ID NO: 129 - protein, 1042 aa, Escherichia coli; identical to SEQ ID NO: 19

SEQ ID NO: 130 - protein, 263 aa, Escherichia coli
MSSNFRHQLLSLSLLVGIAAPWAAFAQAPISSVGSGSVEDRVTQLERISNAHSQLLTQLQQQLSDNQSDIDSLRGQIQENQYQLNQVVERQKQILLQIDSLSSGGAAAQSTSGDQSGVAASTTPTADAGTANAGAPVKSGDANTDYNAAIALVQDKSRQDDAMVAFQNFIKNYPDSTYLPNANYWLGQLNYNKGKKDDAAYYFASVVKNYPKSPKAADAMFKVGVIMQDKGDTAKAKAVYQQVISKYPGTDGAKQAQKRLNAM

SEQ ID NO: 131 - protein, 362 aa, Escherichia coli
MMKRNILAVIVPALLVAGTANAAEIYNKDGNKVDLYGKAVGLHYFSKGNGENSYGGNGDMTYARLGFKGETQINSDLTGYGQWEYNFQGNNSEGADAQTGNKTRLAFAGLKYADVGSFDYGRNYGVVYDALGYTDMLPEFGGDTAYSDDFFVGRVGGVATYRNSNFFGLVDGLNFAVQYLGKNERDTARRSNGDGVGGSISYEYEGFGIVGAYGAADRTNLQEESSLGKGKKAEQWATGLKYDANNIYLAANYGETRNATPITNKFTNTSGFANKTQDVLLVAQYQFDFGLRPSIAYTKSKAKDVEGIGDVDLVNYFEVGATYYFNKNMSTYVDYIINQIDSDNKLGVGSDDTVAVGIVYQF

SEQ ID NO: 132 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDTTGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 133 - protein, 539 aa, Escherichia coli
MAAVCGTSGIASLFSQAAFAADSDIADGQTQRFDFSILQSMAHDLAQTAWRGAPRPLPDTLATMTPQAYNSIQYDAEKSLWHNVENRQLDAQFFHMGMGFRRRVRMFSVDPATHLAREIHFRPELFKYNDAGVDTKQLEGQSDLGFAGFRVFKAPELARRDVVSFLGASYFRAVDDTYQYGLSARGLAIDTYTDSKEEFPDFTAFWFDTVKPGATTFTVYALLDSASITGAYKFTIHCEKNQVIMDVENHLYARKDIKQLGIAPMTSMFSCGTNERRMCDTIHPQIHDSDRLSMWRGNGEWICRPLNNPQKLQFNAYTDNNPKGFGLLQLDRDFSHYQDIMGWYNKRPSLWVEPRNKWGKGTIGLMEIPTTGETLDNIVCFWQPEKAVKAGDEFAFQYRLYWSAQPPVHCPLARVMATRTGMGGFPEGWAPGEHYPEKWARRFAVDFVGGDLKAAAPKGIEPVITLSSGEAKQIEILYIEPIDGYRIQFDWYPTSDSTDPVDMRMYLRCQGDAISETWLYQYFPPAPDKRQYVDDRVMS

SEQ ID NO: 134 - protein, 167 aa, Escherichia coli
MATAGMLLKLNSQMNREFYASNLYLHLSNWCSEQSLNGTATFLRAQAQSNVTQMMRMFNFMKSVGATPIVKAIDVPGEKLNSLEELFQKTMEEYEQRSSTLAQLADEAKELNDDSTVNFLRDLEKEQQHDGLLLQTILDEVRSAKLAGMCPVQTDQHVLNVVSHQLH

SEQ ID NO: 135 - protein, 487 aa, Escherichia coli
MFRQLKKNLVATLIAAMTIGQVAPAFADSADTLPDMGTSAGSTLSIGQEMQMGDYYVRQLRGSAPLINDPLLTQYINSLGMRLVSHANSVKTPFHFFLINNDEINAFAFFGGNVVLHSALFRYSDNESQLASVMAHEISHVTQRHLARAMEDQQRSAPLTWVGALGSILLAMASPQAGMAALTGTLAGTRQGMISFTQQNEQEADRIGIQVLQRSGFDPQAMPTFLEKLLDQARYSSRPPEILLTHPLPESRLADARNRANQMRPIVVQSSEDFYLAKVRTLGMYNSGRNQLTSDLLDEWAKGNVRQQRAAQYGRALQAMEANKYDEARKTLQPLLAAEPGNAWYLDLATDIDLGQNKANDAINRLKNARDLRTDPVLQLNLANAYLQGGQPQEAANILNRYTFNNKDDSNGWDLLAQAEAALNNRDQELAARAEGYALAGRLDQAISLLSSASSQVKLGSLQQARYDARIDQLRQLQERFKPYTKM

SEQ ID NO: 136 - protein, 524 aa, Escherichia coli
MFSTLRRTLFALLACASFIVHAAAPDEITTAWPVNVGPLNPHLYTPNQMFAQSMVYEPLVKYQADGSVIPWLAKSWTHSEDGKTWTFTLRDDVKFSNGEPFDAEAAAENFRAVLDNRQRHAWLELANQIVDVKALSKNELQITLKSAYYPFLQELALPRPFRFIAPSQFKNHETMNGIKAPIGTGPWVLQESKLNQYDVFVRNENYWGEKPAIKKITFNVIPDPTTRAVAFETGDIDLLYGNEGLLPLDTFARFSQNPAYHTQLSQPIETVMLALNTAKAPTNELAVREALNYAVNKKSLIDNALYGTQQVADTLFAPSVPYANLGLKPRQYDPQKAKALLEKAGWTLPAGKDIREKNGQPLRIELSFIGTDALSKSMAEIIQADMRQIGADVSLIGEEESSIYARQRDGRFGMIFHRTWGAPYDPHAFLSSMRVPSHADFQAQQGLADKPLIDKEIGEVLATHDETQRQALYRDILTRLHDEAVYLPISYISMMVVSKPELGNIPYAPIATEIPFEQIKPVKP

SEQ ID NO: 137 - protein, 471 aa, Escherichia coli
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKAMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAYRIAQVQYLVDGLEEIGVVCQQAGGHAAFVDAGKLLPHIPADQFPAQALACELYKVAGIRAVEIGSFLLGRDPKTGKQLPCPAELLRLTIPRATYTQTHMDFIIEAFKHVKENAANIKGLTFTYEPKVLRHFTAKLKEV

SEQ ID NO: 138 - protein, 628 aa, Escherichia coli
MKPAASFFYCGCFTMIKKASLLTACSVTAFSAWAQDTSPDTLVVTANRFEQPRSTVLAPTTVVTRQDIDRWQSTSVNDVLRRLPGVDITQNGGSGQLSSIFIRGTNASHVLVLIDGVRLNLAGVSGSADLSQFPIALVQRVEYIRGPRSAVYGSDAIGGVVNIITTRDHPGTEISAGWGSNSYQNYDVSTQQQLGDKTRVTLLGDYAHTHGYDVVAYGNTGTQAQPDNDGFLSKTLYGALEHNFTDVWSGFVRGYGYDNRTNYDAYYSPGLPLVDTRKLYSQSWDAGLRYNGELIKSQLITSYSHSKDYNYDPHYGRYDSSATLDEMKQYTVQWANNIIIGHGNIGAGVDWQKQSTAPGTAYVEDGYDQRNTGIYLTGLQQVGDFTFEGAGRSDDNSQFGRHGTWQTSAGWEFIEGYRFIASYGTSYKAPNLGQLYGSYGNPNLNPEKSKQWEGAFEGLTAGVNWRISGYRNDVSDLIDYDDHTLKYYNEGKARIKGVEATANFDTGPLTHTVSYDYVDARNAITDTPLLRRAKQQVKYQLDWQLYDFDWGITYQYLGTRYDKDYSSYPYQTVKMGGVSLWDLAVAYPVTSHLTVRGKIANLFDKDYETVYGYQTAGREYTLSGSYTF

SEQ ID NO: 139 - protein, 878 aa, Escherichia coli
MSYLNLRLYQRNTQCLHIRKHRLAGFFVRLFVACAFAAQAPLSSAELYFNPRFLADDPQAVADLSRFENGQELPPGTYRVDIYLNNGYMATRDVTFNTGDSEQGIVPCLTRAQLASMGLNTASVSGMNLLADDACVPLTSMIHDATAQLDVGQQRLNLTIPQAFMSNRARGYIPPELWDPGINAGLLNYNFSGNSVQNRIGGNSHYAYLNLQSGLNIGAWRLRDNTSWSYNSSDSSSGSKNKWQHINTWLERDIIPLRSRLTLGDGYTQGDIFDGINFRGAQLASDDNMLPDSQRGFAPVIHGIARGTAQVTIKQNGYDIYNSTVPPGPFTINDIYAAGNSGDLQVTIKEADGSTQIFTVPYSSVPLLQREGHTRYSITAGEYRSGNAQQEKPRFFQSTLLHGLPAGWTIYGGTQLADRYRAFNFGIGKNMGALGALSVDMTQANSTLPDDSQHDGQSVRFLYNKSLNESGTNIQLVGYRYSTSGYFNFADTTYSRMNGYNIETQDGVIQVKPKFTDYYNLAYNKRGKLQLTVTQQLGRTSTLYLSGSHQTYWGTSNVDEQFQAGLNTAFEDINWTLSYSLTKNAWQKGRDQMLALNVNIPFSHWLRSDSKSQWRHASASYSMSHDLNGRMTNLAGVYGTLLEDNNLSYSVQTGYAGGGDGNSGSTGYATLNYRGGYGNANIGYSHSDDIKQLYYGVSGGVLAHANGVTLGQPLNDTVVLVKAPGAKDAKVENQTGVRTDWRGYAVLPYATEYRENRVALDTNTLADNVDLDNAVANVVPTRGAIVRAEFKARVGIKLLMTLTHNNKPLPFGAMVTSESSQSSGIVADNGQVYLSGMPLAGKVQVKWGEEENAHCVANYQLPPESQQQLLTQLSAECR

SEQ ID NO: 140 - protein, 645 aa, Escherichia coli
VEKAKHVTWRLLAVGVCLLTVSSVARADSLDEQRSRYAQIKQAWDNRQMDVVEQMMPGLKDYPLYPYLEYRQITDDLMNQPAVTVTNFVRANPTLPPARTLQSRFVNELARREDWRGLLAFSPEKPGTTEAQCNYYYAKWNTGQSEEAWQGAKELWLTGKSQPNACDKLFSVWRASGKQDPLAYLERIRLAMKAGNTGLVTVLAGQMPADYQTIASAIISLANNPNTVLTFARTTGATDFTRQMAAVAFASVARQDAENARLMIPSLAQAQQLNEDQIQELRDIVAWRLMGNDVTDEQAKWRDDAIMRSQSTSLIERRVRMALGTGDRRGLNTWLARLPMEAKEKDEWRYWQADLLLERGREAEAKEILHQLMQQRGFYPMVAAQRIGEEYELKIDKAPQNVDSALTQGPEMARVRELMYWNLDNTARSEWANLVKSKSKTEQAQLARYAFNNQWWDLSVQATIAGKLWDHLEERFPLAYNDLFKRYTSGKEIPQSYAMAIARQESAWNPKVKSPVGASGLMQIMPGTATHTVKMFSIPGYSSPGQLLDPETNINIGTSYLQYVYQQFGNNRIFSSAAYNAGPGRVRTWLGNSAGRIDAVAFVESIPFSETRGYVKNVLAYDAYYRYFMGDKPTLMSATEWGRRY

SEQ ID NO: 141 - protein, 314 aa, Escherichia coli
MKLKNTLLASALLSATAFSVNAATELTPEQAAAVKPFDRVVITGRFNAIGEAVKAVSRRADKEGAASFYVVDTSDFGNSGNWRVVADLYKADAEKAEETSNRVINSVVELPKDQAVLIEPFDTVTVQGFYRSQPEVNDAITKAAKAKGAYSFYIVRQIDANQGGNQRITAFIYKKDAKKRIVQSPDVIPADSEAGRAALAAGGEAAKKVEIPGVATTASPSSEVGRFFETQSSKGGRYTVTLPDGTKVEELNKATAAMMVPFDSIKFSGNYGNMTEVSYQVAKRAAKKGAKYYHITRQWQERGNNLTVSADLYK

SEQ ID NO: 142 - protein, 438 aa, Escherichia coli
MADYQGKNVVIIGLGLTGLSCVDFFLARGVTPRVMDTRMTPPGLDKLPEAVERHTGGLNDEWLMAADLIVASPGIALAHPSLSAAADAGIEIVGDIELFCREAQAPIVAITGSNGKSTVTTLVGEMAKAAGVNVGVGGNIGLPALMLLDDECELYVLELSSFQLETTSSLQAVAATILNVTEDHMDRYPFGLQQYRAAKLRIYENAKVCVVNADDALTMPIRGADERCVSFGVNMGDYHLNHQQGETWLRVKGEKVLNVKEMKLSGQHNYTNALAALALADAAGLPRASSLKALTTFTGLPHRFEVVLEHNGVRWVNDSKATNVGSTEAALNGLHVDGTLHLLLGGDGKSADFSPLARYLNGDNVRLYCFGRDGAQLAALRPEVAEQTETMEQAMRLLAPRVQPGDMVLLSPACASLDQFKNFEQRGNEFARLAKELG

SEQ ID NO: 143 - protein, 229 aa, Escherichia coli
MILTDPEWQAVLLSLKVSSLAVLFSLPFGIFFAWLLVRCTFPGKALLDSVLHLPLVLPPVVVGYLLLVSMGRRGFIGERLYDWFGLSFAFSWRGAVLAAAVMSFPLMVRAIRLALEGVDIKLEQAARTLGAGRWRVFFTITLPLTLPGIIVGTVLAFARSLGEFGATITFVSNIPGETRTIPSAMYTLIQTPGGESGAARLCIISIALAMISLLISEWLARISRERAGR

SEQ ID NO: 144 - protein, 363 aa, Escherichia coli
MKIGVFVPIGNNGWLISTHAPQYMPTFELNKAIVQKAEHYHFDFALSMIKLRGFGGKTEFWDHNLESFTLMAGLAAVTSRIQIYATAATLTLPPAIVARMAATIDSISGGRFGVNLVTGWQKPEYEQMGIWPGDDYFSRRYDYLTEYVQVLRDLWGSGKSDFKGDFFTMDDCRVSPQPSVPMKVICAGQSDAGMAFSARYADFNFCFGKGVNTPTAFAPTAARMKQAAEQTGRDVGSYVLFMVIADETDDAARAKWEHYKAGADEEALSWLTEQSQKDTRSGTDTNVRQMADPTSAVNINMGTLVGSYASVARMLDEVASVPGAEGVLLTFDDFLSGIETFGERIQPLMQCRAHLPALTQEVA

SEQ ID NO: 145 - protein, 78 aa, Escherichia coli
MKATKLVLGAVILGSTLLAGCSSNAKIDQLSSDVQTLNAKVDQLSNDVNAMRSDVQAAKDDAARANQRLDNMATKYRK

SEQ ID NO: 146 - protein, 518 aa, Escherichia coli
MEFLMDPSIWAGLLTLVVLEIVLGIDNLVFIAILADKLPPKQRDKARLLGLSLALIMRLGLLSLISWMVTLTKPLFTVMDFSFSGRDLIMLFGGIFLLFKATTELHERLENRDHDSGHGKGYASFWVVVTQIVILDAVFSLDAVITAVGMVNHLPVMMAAVVIAMAVMLLASKPLTRFVNQHPTVVVLCLSFLLMIGLSLVAEGFGFHIPKGYLYAAIGFSIIIEVFNQIARRNFIRHQSTLPLRARTADAILRLMGGKRQANVQHDADNPMPMPIPEGAFAEEERYMINGVLTLASRSLRGIMTPRGEISWVDANLGVDEIREQLLSSPHSLFPVCRGELDEIIGIVRAKELLVALEEGVDVAAIASASPAIIVPETLDPINLLGVLRRARGSFVIVTNEFGVVQGLVTPLDVLEAIAGEFPDADETPEIITDGDGWLVKGGTDLHALQQALDVEHLADDDDIATVAGLVISANGHIPRVGDVIDVGPLHITIIEANDYRVDLVRIVKEQPAHDEDE

SEQ ID NO: 147 - protein, 446 aa, Escherichia coli
MSQKTLFTKSALAVAVALISTQAWSAGFQLNEFSSSSLGRAYSGEGAIADDAGNVSRNPALITMFDRPTFSAGAVYIDPDVNISGTSPSGRSLKADNIAPTAWVPNMHFVAPINDQFGWGASITSNYGLATEFNDTYAGGSVGGTTDLETMNLNLSGAYRLNNAWSFGLGFNAVYARAKIERFAGDLGQLVAGQIMQSPAGQTQQGQALAATANGIDSNTKIAHLNGNQWGFGWNAGILYELDKNNRYALTYRSEVKIDFKGNYSSDLNRAFNNYGLPIPTATGGATQSGYLTLNLPEMWEVSGYNRVAPQWAIHYSLAYTSWSQFQQLKATSTSGDTLFQKHEGFKDAYRIALGTTYYYDDNWTFRTGIAFDDSPVPAQNRSISIPDQDRFWLSAGTTYAFNKDASVDVGVSYMHGQSVKINEGPYQFESEGKAWLFGTNFNYAF

SEQ ID NO: 148 - protein, 446 aa, Escherichia coli
MMITLRKLPLAVAVAAGVMSAQAMAVDFHGYARSGIGWTGSGGEQQCFQTTGAQSKYRLGNECETYAELKLGQEVWKEGDKSFYFDTNVAYSVAQKNDWEATDPAFREANVQGKNLIEWLPGSTIWAGKRFYQRHDVHMIDFYYWDISGPGAGLENIDVGFGKLSLAATRSSEAGGSSSFASNNIYDYTNETANDVFDVRLAQMEINPGGTLELGVDYGRANLRDNYRLVDGASKDGWLFTAEHTQSVLKGFNKFVVQYATDSMTSQGKGLSQGSGVAFDNEKFAYNINNNGHMLRILDHGAISMGDNWDMMYVGMYQDINWDNDNGTKWWTVGIRPMYKWTPIMSTVMEIGYDNVESQRTGDKNNQYKITLAQQWQAGDSIWSRPAIRVFATYAKWDEKWGYDYTGSSSTNPYYGKAVSADFNGGSFGRGDSDEWTFGAQMEIWW

SEQ ID NO: 149 - protein, 237 aa, Escherichia coli
VNEAESQARAIVDQARNGADFGKLAIAHSADQQALNGGQMGWGRIQELPGIFAQALSTAKKGDIVGPIRSGVGFHILKVNDLRGESKNISVTEVHARHILLKPSPIMTDEQARVKLEQIAADIKSGKTTFAAAAKEFSQDPGSANQGGDLGWATPDIFDPAFRDALTRLNKGQMSAPVHSSFGWHLIELLDTRNVDKTDAAQKDRAYRMLMNRKFSEEAASWMQEQRASAYVKILSN

SEQ ID NO: 150 - protein, 191 aa, Escherichia coli
MKNWKTLLLGIAMIANTSFAAPQVVDKVAAVVNNGVVLESDVDGLMQSVKLNAAQARQQLPDDATLRHQIMERLIMDQIILQMGQKMGVKISDEQLDQAIANIAKQNNMTLDQMRSRLAYDGLNYNTYRNQIRKEMIISEVRNNEVRRRITILPQEVEILAQQVGNQNDASTELNLSHILNPLAEKPTLIR

SEQ ID NO: 151 - protein, 630 aa, Escherichia coli
MAIEIKVPDIGADEVEITEILVKVGDKVEAEQSLITVEGDKASMEVPSPQAGIVKEIKVSVGDKTQTGALIMIFDSADGAADAAPAQAEEKKEAAPAAAPAAAAAKDVNVPDIGSDEVEVTEILVKVGDKVEAEQSLITVEGDKASMEVPAPFAGTVKEIKVNVGDKVSTGSLIMIFEVAGEAGAAAPAAKQEAAPAAAPAPAAGVKEVNVPDIGGDEVEVTEVMVKVGDKVAAEQSLITVEGDKASMEVPAPFAGVVKELKVNVGDKVKTGSLIMIFEVEGAAPAAAPAKQEAAAPAPAAKAEAPAAAPAAKAEGKSEFAENDAYVHATPLIRRLAREFGVNLAKVKGTGRKGRILREDVQAYVKEAIKRAEAAPAATGGGIPGMLPWPKVDFSKFGEIEEVELGRIQKISGANLSRNWVMIPHVTHFDKTDITELEAFRKQQNEEAAKRKLDVKITPVVFIMKAVAAALEQMPRFNSSLSEDGQRLTLKKYINIGVAVDTPNGLVVPVFKDVNKKGIIELSRELMTISKKARDGKLTAGEMQGGCFTISSIGGLGTTHFAPIVNAPEVAILGVSKSAMEPVWNGKEFVPRLMLPISLSFDHRVIDGADGARFITIINNTLSDIRRLVM

SEQ ID NO: 152 - protein, 550 aa, Escherichia coli
MKLLQRGVALALLTTFTLASETALAYEQDKTYKITVLHTNDHHGHFWRNEYGEYGLAAQKTLVDGIRKEVAAEGGSVLLLSGGDINTGVPESDLQDAEPDFRGMNLVGYDAMAIGNHEFDNPLTVLRQQEKWAKFPLLSANIYQKSTGERLFKPWALFKRQDLKIAVIGLTTDDTAKIGNPEYFTDIEFRKPADEAKLVIQELQQTEKPDIIIAATHMGHYDNGEHGSNAPGDVEMARALPARSLAMIVGGHSQDPVCMAAENKKQVDYVPGTPCKPDQQNGIWIVQAHEWGKYVGRADFEFRNGEMKMVNYQLIPVNLKKKVTWEDGKSERVLYTPEIAENQQMISLLSPFQNKGKAQLEVKIGETNGRLEGDRDKVRFVQTNMGRLILAAQMDRTGADFAVMSGGGIRDSIEAGDISYKNVLKVQPFGNVVVYADMTGKEVIDYLTAVAQMKPDSGAYPQFANVSFVAKDGKLNDLKIKGEPVDPAKTYRMATLNFNATGGDGYPRLDNKPGYVNTGFIDAEVLKAYIQKSSPLDVSVYEPKGEVSWQ

SEQ ID NO: 153 - protein, 430 aa, Escherichia coli
MKQALRVAFGFLILWASVLHAEVRIVIDSGVDSGRPIGVVPFQWAGPGAAPEDIGGIVAADLRNSGKFNPLDRARLPQQPGSAQEVQPAAWSALGIDAVVVGQVTPNPDGSYNVAYQLVDTGGAPGTVLAQNSYKVNKQWLRYAGHTASDEVFEKLTGIKGAFRTRIAYVVQTNGGQFPYELRVSDYDGYNQFVVHRSPQPLMSPAWSPDGSKLAYVTFESGRSALVIQTLANGAVRQVASFPRHNGAPAFSPDGSKLAFALSKTGSLNLYVMDLASGQIRQVTDGRSNNTEPTWFPDSQNLAFTSDQAGRPQVYKVNINGGAPQRITWEGSQNQDADVSSDGKFMVMVSSNGGQQHIAKQDLATGGVQVLSSTFLDETPSLAPNGTMVIYSSSQGMGSVLNLVSTDGRFKARLPATDGQVKFPAWSPYL

SEQ ID NO: 154 - protein, 112 aa, Escherichia coli
MNKNMAGILSAAAVLTMLAGCTAYDRTKDQFVQPVVKDVKKGMSRAQVAQIAGKPSSEVSMIHARGTCQTYILGQRDGKAETYFVALDDTGHVINSGYQTCAEYDTDPQATK

SEQ ID NO: 155 - protein, 191 aa, Escherichia coli
MKSLRLMLCAMPLMLTGCSTMSSVNWSAANPWNWFGSSTKVSEQGVGELTASTPLQEQAIADALDGDYRLRSGMKTANGNVVRFFEVMKGDNVAMVINGDQGTISRIDVLDSDIPADTGVKIGTPFSDLYSKAFGNCQKADSDDNRAVECKAEGSQHISYQFSGEWSGPEGLMPSDDTLKNWKVSKIIWRR

SEQ ID NO: 156 - protein, 230 aa, Escherichia coli
MAAQANELPDGPHIVTSGTASVDAVPDIATLAIEVNVAAKDAATAKKQADERVAQYISFLELNQIAKKDISSANLRTQPDYDYQDGKSILKGYRAVRTVEVTLRQLDKLNSLLDGALKAGLNEIRSVSLGVAQPDAYKDKARKAAIDNAIHQAQELANGFHRKLGPVYSVRYHVSNYQPSPMVRMMKADAAPVSAQETYEQAAIQFDDQVDVVFQLEPVDQPPAKTPAAH

SEQ ID NO: 157 - protein, 130 aa, Escherichia coli
MKKFAAVIAVMALCSAPVMAAEQGGFSGPSATQSQAGGFQGPNGSVTTVESAKSLRDDTWVTLRGNIVERISDDLYVFKDASGTINVDIDHKRWNGVTVTPKDTVEIQGEVDKDWNSVEIDVKQIRKVNP

SEQ ID NO: 158 - protein, 127 aa, Escherichia coli [sequence omitted]

SEQ ID NO: 159 - protein, 206 aa, Escherichia coli
MARYLGPKLKLSRREGTDLFLKSGVRAIDTKCKIEQAPGQHGARKPRLSDYGVQLREKQKVRRIYGVLERQFRNYYKEAARLKGNTGENLLALLEGRLDNVVYRMGFGATRAEARQLVSHKAIMVNGRVVNIASYQVSPNDVVSIREKAKKQSRVKAALELAEQREKPTWLEVDAGKMEGTFKRKPERSDLSADINEHLIVELYSK

SEQ ID NO: 160 - protein, 188 aa, Escherichia coli
MNMTKGALILSLSFLLAACSSIPQNIKGNNQPDIQKSFVAVHNQPGLYVGQQARFGGKVINVINGKTDTLLEIAVLPLDSYAKPDIEANYQGRLLARQSGFLDPVNYRNHFVTILGTIQGEQPGFINKVPYNFLEVNMQGIQVWHLREVVNTTYNLWDYGYGAFWPEPGWGAPYYTNAVSQVTPELVK

SEQ ID NO: 161 - protein, 548 aa, Escherichia coli
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVAAAVEELKTLSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMGGMGGMM

SEQ ID NO: 162 - protein, 201 aa, Escherichia coli
MTMTRLKISKTLLAVMLTSAVATGSAYAENNAQTTNESAGQKVDSSMNKVGNFMDDSAITAKVKAALVDHDNIKSTDISVKTDQKVVTLSGFVESQAQAEEAVKVAKGVEGVTSVSDKLHVRDAKEGSVKGYAGDTATTSEIKAKLLADDIVPSRHVKVETTDGVVQLSGTVDSQAQSDRAESIAKAVDGVKSVKNDLKTK

SEQ ID NO: 163 - protein, 749 aa, Escherichia coli
MNKKIHSLALLVNLGIYGVALPVLAEETSDNAVSHDDTIVVTAAEQNLQAPGVSTITADEIRKNPVARDVSEIIRTMPGVNLTGNSTSGQRGNNRQIDIRGMGPENTLILIDGKPVSSRNSVRQGWRGERDTRGDTSWVPPEMIERIEVLRGPAAARYGNGAAGGVVNIITKKGSGEWHGSWDAYFNAPEHKEEGATKRTNFSLTGPLGDEFSFRLYGNLDKTQADAWDINQGHQSARAGTYATTLPAGREGVINKDINGVVRWDFAPLQSLELEAGYSRQGNLYAGDTQNTNSDAYTRSKYGDETNRLYRQNYSLTWNGGWDNGVTTSNWVQYEHTRNSRIPEGLAGGTEGKFNEKAAQDFVDIDLDDVMLHSEVNLPIDFLVNQTLTLGTEWNQQRMKDLSSNTQALTGTNTGGAIDGVSATDRSPYSKAEIFSLFAENNMELTDSTIVTPGLRFDHHSIVGNNWSPALNISQSLGDDFTLKMGIARAYKAPSLYQTNPNYILYSKGQGCYASAGGCYLQGNDDLKAETSINKEIGLEFKRDGWLAGVTWFRNDYRNKIEAGYVAVGQNAVGTDLYQWDNVPKAVVEGLEGSLNVPVSETVMWTNNITYMLKSENKTTGDRLSIIPEYTLNSTLSWQAREDLSMQTTFTWYGKQQPKKYNYKGQPAVGPETKEISPYSIVGLSATWDVTKNVSLTGGVDNLFDKRLWRAGNAQTTGDLAGANYIAGAGAYTYNEPGRTWYMSINTHF

SEQ ID NO: 164 - protein, 350 aa, Escherichia coli [sequence omitted]

SEQ ID NO: 165 - protein, 714 aa, Escherichia coli
VSRIIMLIPTGTSVGLTSVSLGVIRAMERKGVRLSVFKPIAQPRTGGDAPDQTTTIVRANSSTTTAAEPLKMSYVEGLLSSNQKDVLMEEIIANYHANTKDAEVVLVEGLVPTRKHQFAQSLNYEIAKTLNAEIVFVMSQGTDTPEQLKERIELTRNSFGGAKNTNITGVIVNKLNAPVDEQGRTRPDLSEIFDDSTKAKVNNVDPAKLQESSPLPVLGAVPWSFDLIATRAIDMARHLNATIINEGDINTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYINADWIDSLTATSERSRRLSPPAFRYQLTELARKAGKRIVLPEGDEPRTVKAAAICAERGIATCVLLGNPAEINRVAASQGVELGAGIEIVDPEVVRENYVGRLVELRKNKGMTETVAREQLEDNVVLGTLMLEQDEVDGLVSGAVHTTANTIRPPLQLIKTAPGSSLVSSVFFMLLPEQVYVYGDCAINPDPTAEQLAEIAIQSADSAAAFGIEPRVAMLSYSTGTSGAGSDVEKVREATRLAQEKRPDLMIDGPLQYDAAVIADVAKSKAPNSPVAGRATVFIFPDLNTGNTTYKAVQRSADLISIGPMLQGMRKPVNDLSRGALVDDIVYTIALTAIQSAQQQ

SEQ ID NO: 166 - protein, 195 aa, Escherichia coli
MRLQFSALFFFLYCVFIRGVFADPFPPPGMSLPEYWGEEHVWWDGRAAFHGEVVRPACTLAMEDAWQIIDMGETPVRDLQNGFSGPERKFSLRLRNCEFNSQGGNLFSDSRIRVTFDGVRGETPDKFNLSGQAKGINLQIADARGNIARAGKVMPAIPLTGNEEALDYTLRIVRNGKKLEAGNYFAVLGFRVDYE

SEQ ID NO: 167 - protein, 170 aa, Escherichia coli
MNMNNPLEVLGHVSWLWASSPLHRNWPVSLFAINVLPAIRANQYALLTRDNYPVAYCSWANLSLENEIKYLNDVTSLVAEDWTSGDRKWFIDWIAPFGDNGALYKYMRKKFPDELFRAIRVDPKTHVGKVSEFHGGKIDKQLANKIFKQYHHELITEVKNKSDFNFSLTG

SEQ ID NO: 168 - protein, 449 aa, Escherichia coli
ADITEIETTTGEKKNTNVTCPADPGKLSPEELKRLPSECSSVVEQNLMPWLVTGAATALITTLAIVELNDDDDHHRNNSPLPPTPPDDDSDDTPVPPTPGGDEIIPDDGPDDTPAPPKPIAFNNDVTLDKTAKTLTIRDSVFSYTENADGTISLQDSNGRKATINLWQIDETNNTVALEGMSADGATKWQYNHNGELVITGDNTTVNNTGKTIVDGKGATGTEIAGNNAVVNQDGELDVSGGGHGIDITGDSATVDNKGGMTVTDPDSIGIQIDGDKAVVNNDGDNAISNGGTGTQVNGDEATVNNNGSTTVDGKDSTGTEINGDKAIVNNDGDSTILDGGTETRITGDDATANNSGNTTVDGQGSTGTEIAGNNAVVNQDGELDVSGGGHGIDITGDSATVDNKGGMTVIDPDSIGIQIDGDKAVVNNDGDNAISNGGTGTQINGDEA